(19) United States
(12) Patent Application Publication
(10) Pub. No.: US 2021/0069262 A1
(43) Pub. Date: Mar. 11, 2021

(54) COMPOSITIONS COMPRISING CO-SELECTED MICROBIOTA AND METHODS FOR USE THEREOF

(71) Applicant: Nubiyota LLC, Teaneck, NJ (US)

(72) Inventor: Emma Allen-Vercoe, Guelph (CA)

(21) Appl. No.: 16/960,233

(22) PCT Filed: Jan. 4, 2019
(86) PCT No.: PCT/US2019/012376

§ 371 Date: Jul. 6, 2020

Related U.S. Application Data
(60) Provisional application No. 62/614,151, filed on Jan. 5, 2018.
(60) Provisional application No. 62/683,850, filed on Jun. 12, 2018.

(51) Int. Cl. A61K 35/741; A61K 35/742; A61K 35/745; A61K 35/747; A61K 47/38; A61K 9/48; A61P 1/00

(52) U.S. Cl. CPC A61K 35/741; A61K 35/742; A61K 35/745; A61K 35/747; A61K 2035/115; A61K 9/4808; A61K 9/4866; A61P 1/00; A61K 47/38

(57) ABSTRACT

Anhydrous compositions comprising a co-selected microbiota and methods for using same to treat disorders associated with dysbiosis (an imbalance of the microbial community inhabiting a subject or inhabiting a particular tissue in a subject) are described herein. In particular, anhydrous compositions comprising a co-selected microbiota and methods for treating gastrointestinal disorders associated with dysbiosis are envisioned. The use of such anhydrous compositions comprising a co-selected microbiota for treating disorders associated with dysbiosis (e.g., gastrointestinal disorders associated with dysbiosis) and the use of such anhydrous compositions comprising a co-selected microbiota in the preparation of a medicament for treating disorders associated with dysbiosis (e.g., gastrointestinal disorders associated with dysbiosis) are also embodied herein.

### RELATED APPLICATIONS

This application claims priority of U.S. Provisional Application No. 62/614,151, filed Jan. 5, 2018 and U.S. Provisional Application No. 62/683,850, filed Jun. 12, 2018, the entirety of which are incorporated herein by reference for all purposes.

### SEQUENCE LISTING

The instant application contains a Sequence Listing which has been submitted electronically in ASCII format and is hereby incorporated by reference in its entirety.

### FIELD OF THE INVENTION

The field of invention relates to compositions and methods for treating disorders associated with dysbiosis (an imbalance of the microbial community inhabiting a subject or inhabiting a particular tissue in a subject). In particular, compositions and methods for treating gastrointestinal disorders associated with dysbiosis are envisioned.

### BACKGROUND OF THE INVENTION

Dysbiosis is associated with a variety of diseases and disorders. Accordingly, there is a need for reagents and methods for using same to restore a healthful balance of microorganisms that comprise a healthy microbiome.

### SUMMARY

Microbial Ecosystem Therapeutic (designated MET-2) is described herein. Exemplary subgroups of MET-2 (e.g., MET-2A and MET-2B) are also set forth herein. Additional exemplary subgroups of MET-2, MET-2A, and MET-2B are set forth in, e.g., Tables 3-5 presented herein. Further exemplary subgroups of MET-2 include: NB2B-6-CNA, NB2A-9-NA, NB2A-14-FMU, NB2A-8-WC, NB2A-12-BBE, NB2B-16-TSAB, NB2B-11-FAA, NB2B-13-DCM, NB2A-2-FAA, NB2A-3-NA, NB2B-BHI-1, NB2A-17-FMU, NB2B-19-DCM, NB2B-AER-MRS-02, and NB2A-10-MRS of Table 1 and also NB2B-20-GAM, NB2B-6-CNA, NB2A-9-NA, 14 LG, NB2A-8-WC, NB2A-12-BBE, NB2A-3-NA, NB2A-17-FMU, NB2B-19-DCM, NB2B-10-FAA, NB2B-26-FMU of Table 1. As described herein at least one species of MET-2 and exemplary subgroups thereof and compositions comprising at least one species of MET-2 and exemplary subgroups thereof are encompassed, wherein the total number of species of MET-2 or a subgroup thereof consists of the total number of species included in MET-2 or the specific subgroup indicated. In certain embodiments, the subset of bacterial species listed in Table 1 consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 species. In certain embodiments, an anhydrous composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 of the bacterial species listed in Table 1. Accordingly, at least one species of MET-2 and at least one species of exemplary subgroups of MET-2 and compositions comprising at least one species of MET-2 and at least one species of exemplary subgroups MET-2 are presented as therapeutic agents for use in treating a variety of gastrointestinal diseases (e.g., ulcerative colitis). Methods for treating a variety of gastrointestinal diseases by administering at least one bacterial species of MET-2 and/or at least one bacterial species of exemplary subgroups of MET-2 or compositions comprising at least one bacterial species of MET-2 and/or at least one bacterial species of exemplary subgroups of MET-2 to a subject in need thereof are also described herein. Also encompassed are the use of at least one bacterial species of MET-2 and/or at least one bacterial species of exemplary subgroups of MET-2 for treating a variety of gastrointestinal diseases and the use of at least one bacterial species of MET-2 and/or at least one bacterial species of exemplary subgroups of MET-2 in the preparation of a medicament for treating a variety of gastrointestinal diseases. Such gastrointestinal diseases include diseases or disorders associated with dysbiosis such as, for example, Clostridium difficile (Clostridioides difficile) infection, Crohn's disease, irritable bowel syndrome (IBS) or spastic colon, idiopathic ulcerative colitis, mucous colitis, collagenous colitis, inflammatory bowel disease in general, microscopic colitis, antibiotic-associated colitis, idiopathic or simple constipation, diverticular disease, and/or AIDS enteropathy.

In an aspect, an anhydrous composition comprising a co-selected microbiota is presented, wherein the co-selected microbiota comprises a plurality of bacterial species consisting of each of the bacterial species listed in Table 1, and optionally, at least one additional bacterial species, wherein the bacterial species listed in Table 1 are in powder-form, wherein the powder-form has a moisture content of less than 5% wt/wt in the anhydrous composition, and wherein the co-selected microbiota exhibits resistance to perturbational stress.

In another aspect, an anhydrous composition comprising a co-selected microbiota is presented, wherein the co-selected microbiota comprises at least one of the bacterial species listed in Table 1, wherein the co-selected microbiota consists of bacterial species recited in Table 1, and optionally, at least one additional bacterial species, wherein the bacterial species listed in Table 1 are in powder-form, wherein the powder-form has a moisture content of less than 5% wt/wt in the anhydrous composition, and wherein the co-selected microbiota exhibits resistance to perturbational stress.

In another aspect, an anhydrous composition comprising a co-selected microbiota is presented, wherein the co-selected microbiota comprises a plurality of bacterial species, the plurality of bacterial species consisting of at least one bacterial species from each phylum of bacteria listed in Table 1, and optionally, at least one additional bacterial species, wherein the bacterial species listed in Table 1 are in powder-form, wherein the powder-form has a moisture content of less than 5% wt/wt in the anhydrous composition, and wherein the co-selected microbiota exhibits resistance to perturbational stress.

In another aspect, an anhydrous composition comprising a co-selected microbiota in presented, wherein the co-selected microbiota comprises at least one of the MET-2A bacterial species listed in Table 3, wherein the co-selected microbiota consists of MET-2A bacterial species recited in Table 3, and optionally, at least one additional bacterial species, wherein the MET-2A bacterial species listed in Table 3 are in powder-form, wherein the powder-form has a moisture content of less than 5% wt/wt in the anhydrous composition, and wherein the co-selected microbiota exhibits resistance to perturbational stress.

In another aspect, an anhydrous composition comprising a co-selected microbiota is presented, wherein the co-selected microbiota comprises at least one of the MET-2B bacterial species listed in Table 3, wherein the co-selected microbiota consists of MET-2B bacterial species recited in Table 3, and optionally, at least one additional bacterial species, wherein the MET-2B bacterial species listed in Table 3 are in powder-form, wherein the powder-form has a moisture content of less than 5% wt/wt in the anhydrous composition, and wherein the co-selected microbiota exhibits resistance to perturbational stress.

In another aspect, an anhydrous composition comprising a co-selected microbiota is presented, wherein the co-selected microbiota comprises at least one of the bacterial species listed in Table 3 that is present in each of MET-2, MET-2A, and MET-2B, wherein the co-selected microbiota consists of the total number of bacterial species listed in Table 3 that are present in each of MET-2, MET-2A, and MET-2B, and optionally, at least one additional bacterial species, wherein the bacterial species present in each of MET-2, MET-2A, and MET-2B are in powder-form, wherein the powder-form has a moisture content of less than 5% wt/wt in the anhydrous composition, and wherein the co-selected microbiota exhibits resistance to perturbational stress.

In another aspect, an anhydrous composition comprising a co-selected microbiota is presented, wherein the co-selected microbiota comprises at least one of the bacterial species listed in Table 1, wherein the co-selected microbiota consists of bacterial species NB2B-20-GAM, NB2B-6-CNA, NB2A-9-NA, 14 LG, NB2A-8-WC, NB2A-12-BBE, NB2A-3-NA, NB2A-17-FMU, NB2B-19-DCM, NB2B-10-FAA, NB2B-26-FMU recited in Table 1, and optionally, at least one additional bacterial species, wherein the bacterial species NB2B-20-GAM, NB2B-6-CNA, NB2A-9-NA, 14 LG, NB2A-8-WC, NB2A-12-BBE, NB2A-3-NA, NB2A-17-FMU, NB2B-19-DCM, NB2B-10-FAA, NB2B-26-FMU recited in Table 1 are in powder-form, wherein the powder-form has a moisture content of less than 5% wt/wt in the anhydrous composition, and wherein the co-selected microbiota exhibits resistance to perturbational stress.

In an embodiment of any one of the aforementioned aspects, the co-selected microbiota comprises at least 25% Gram-negative bacterial species. In an embodiment of any one of the aforementioned aspects, the co-selected microbiota comprises at least 50% Gram-positive bacterial species. In an embodiment of any one of the aforementioned aspects, the co-selected microbiota comprises at least 65% bacterial species within the Firmicutes phylum. In an embodiment of any one of the aforementioned aspects, the co-selected microbiota comprises at least 5% bacterial species within the Bacteroidetes phylum.

In another aspect, an anhydrous composition comprising a co-selected microbiota is presented, wherein the co-selected microbiota comprises at least one of the bacterial species of any one of the following sub-groups described herein, including: NB2B-6-CNA, NB2A-9-NA, NB2A-14-FMU, NB2A-8-WC, NB2A-12-BBE, NB2B-16-TSAB, NB2B-11-FAA, NB2B-13-DCM, NB2A-2-FAA, NB2A-3-NA, NB2B-BHI-1, NB2A-17-FMU, NB2B-19-DCM, NB2B-AER-MRS-02; a sub-group described in Table 3; a sub-group described in Table 4; or a sub-group described in Table 4, and optionally, at least one additional bacterial species, wherein the sub-group of bacterial species are in powder-form, wherein the powder-form has a moisture content of less than 5% wt/wt in the anhydrous composition, and wherein the co-selected microbiota exhibits resistance to perturbational stress.

In a further embodiment of any one of the above aspects or embodiments, the bacterial species are in a state of suspended animation. In a further embodiment of any one of the above aspects or embodiments, the bacterial species exhibit robustness when challenged by perturbational stress in a chemostat model test or an ecosystem output assay.

In a further embodiment of any one of the above aspects or embodiments, the anhydrous composition further comprises a pharmaceutically acceptable carrier. More particularly, the pharmaceutically acceptable carrier is cellulose. More particularly still, the anhydrous composition is encapsulated in a capsule (e.g., the anhydrous composition may be encapsulated in a double capsule).

In a further embodiment of any one of the above aspects or embodiments, the at least one additional bacterial species is a species in the Acidaminococcus genus. More particularly, the Acidaminococcus genus is Acidaminococcus intestini or Acidaminococcus fermentans.

In a further embodiment of any one of the above aspects or embodiments, the anhydrous composition further comprises a prebiotic.

Also encompassed herein is a method for treating a mammalian subject afflicted with a disease or disorder associated with dysbiosis, the method comprising: administering a therapeutically effective amount of an anhydrous composition of any one of the above aspects or embodiments to the mammalian subject, wherein the therapeutically effective amount improves relative ratios of microorganisms in the mammalian subject, thereby treating the mammalian subject. In a particular embodiment thereof, the disease or disorder associated with dysbiosis is Clostridium difficile (Clostridioides difficile) infection, Crohn's disease, irritable bowel syndrome (IBS) or spastic colon, idiopathic ulcerative colitis, mucous colitis, collagenous colitis, inflammatory bowel disease in general, microscopic colitis, antibiotic-associated colitis, idiopathic or simple constipation, diverticular disease, or AIDS enteropathy.

Also encompassed herein is an anhydrous composition of any one of the above aspects or embodiments for use in treating a disease or disorder associated with dysbiosis, wherein the anhydrous composition improves relative ratios of microorganisms. In a particular embodiment thereof, the disease or disorder associated with dysbiosis is Clostridium difficile (Clostridioides difficile) infection, Crohn's disease, irritable bowel syndrome (IBS) or spastic colon, idiopathic ulcerative colitis, mucous colitis, collagenous colitis, inflammatory bowel disease in general, microscopic colitis, antibiotic-associated colitis, idiopathic or simple constipation, diverticular disease, or AIDS enteropathy.

Also encompassed herein is an anhydrous composition of any one of the above aspects or embodiments for use in the preparation of a medicament for treating a disease or disorder associated with dysbiosis, wherein the anhydrous composition improves relative ratios of microorganisms. In a particular embodiment thereof, the disease or disorder associated with dysbiosis is Clostridium difficile (Clostridioides difficile) infection, Crohn's disease, irritable bowel syndrome (MS) or spastic colon, idiopathic ulcerative colitis, mucous colitis, collagenous colitis, inflammatory bowel disease in general, microscopic colitis, antibiotic-associated colitis, idiopathic or simple constipation, diverticular disease, or AIDS enteropathy.

In another aspect, an anhydrous composition comprising a plurality of bacterial species is presented, the plurality of bacterial species consisting of each of the bacterial species listed in Table 1, and optionally, at least one additional bacterial species, wherein the bacterial species listed in Table 1 are (a) in powder-form, wherein the powder-form has a moisture content of less than 5% wt/wt in the anhydrous composition, and wherein the anhydrous composition when tested by a chemostat model test is: (b) suspended in a first growth media and cultured to achieve steady state growth of the plurality of bacterial species in the first growth media, wherein a relative abundance of the plurality of bacterial species at steady state growth in the first growth media is established as a first relative abundance, and (c) the plurality of bacterial species at steady state growth in the first growth media is challenged by perturbational stress, wherein the perturbational stress is a change in at least one of substrate type, substrate availability, or xenobiotic challenge, and the plurality of bacterial species exhibits robustness when challenged by the perturbational stress, wherein the robustness is exhibited by maintenance of the first relative abundance of the plurality of bacterial species after challenge by the perturbational stress.

In another aspect, an anhydrous composition comprising a plurality of bacterial species is presented, the plurality of bacterial species consisting of each of the bacterial species listed in Table 1, and optionally, at least one additional bacterial species, wherein the bacterial species listed in Table 1 are (a) in powder-form, wherein the powder-form has a moisture content of less than 5% wt/wt in the anhydrous composition, and wherein the anhydrous composition when tested by an ecosystem output assay is: (b) suspended in a first growth media and cultured to achieve steady state growth of the plurality of bacterial species in the first growth media, wherein a relative abundance of the plurality of bacterial species at steady state growth in the first growth media is established as a first relative abundance, and (c) the plurality of bacterial species at steady state growth in the first growth media is challenged by perturbational stress, wherein the perturbational stress is a change in at least one of substrate type, substrate availability, or xenobiotic challenge, and the plurality of bacterial species exhibits robustness when challenged by the perturbational stress, wherein the robustness is exhibited by maintenance of functional output of types and quantities of selected small molecules generated by the plurality of bacterial species after challenge by the perturbational stress.

In another aspect, an anhydrous composition comprising a plurality of bacterial species is presented, the plurality of bacterial species consisting of at least one bacterial species from each phylum of bacteria listed in Table 1, and optionally, at least one additional bacterial species, wherein the at least one bacterial species from each phylum of bacteria listed in Table 1 are (a) in powder-form, wherein the powder-form has a moisture content of less than 5% wt/wt in the anhydrous composition, and wherein the anhydrous composition when tested by a chemostat model test is: (b) suspended in a first growth media and cultured to achieve steady state growth of the plurality of bacterial species in the first growth media, wherein a relative abundance of the plurality of bacterial species at steady state growth in the first growth media is established as a first relative abundance, and (c) the plurality of bacterial species at steady state growth in the first growth media is challenged by perturbational stress, wherein the perturbational stress is a change in at least one of substrate type, substrate availability, or xenobiotic challenge, and the plurality of bacterial species exhibits robustness when challenged by the perturbational stress, wherein the robustness is exhibited by maintenance of the first relative abundance of the plurality of bacterial species after challenge by the perturbational stress.

In another aspect, an anhydrous composition comprising a plurality of bacterial species is presented, the plurality of bacterial species consisting of at least one bacterial species from each phylum of bacteria listed in Table 1, and optionally, at least one additional bacterial species, wherein the at least one bacterial species from each phylum of bacteria listed in Table 1 are (a) in powder-form, wherein the powder-form has a moisture content of less than 5% wt/wt in the anhydrous composition, and wherein the anhydrous composition when tested by an ecosystem output assay is: (b) suspended in a first growth media and cultured to achieve steady state growth of the plurality of bacterial species in the first growth media, wherein a relative abundance of the plurality of bacterial species at steady state growth in the first growth media is established as a first relative abundance, and (c) the plurality of bacterial species at steady state growth in the first growth media is challenged by perturbational stress, wherein the perturbational stress is a change in at least one of substrate type, substrate availability, or xenobiotic challenge, and the plurality of bacterial species exhibits robustness when challenged by the perturbational stress, wherein the robustness is exhibited by maintenance of functional output of types and quantities of selected small molecules generated by the plurality of bacterial species after challenge by the perturbational stress.

In an embodiment of each of the above, the bacterial species are in a state of suspended animation. In a further embodiment, the anhydrous composition further comprises a pharmaceutically acceptable carrier (e.g., cellulose). In a further embodiment, the anhydrous composition is encapsulated in a capsule (e.g., a double capsule). In another embodiment, the at least one additional bacterial species is a species in the Acidaminococcus genus (e.g., Acidaminococcus intestini or Acidaminococcus fermentans). In a further embodiment, the anhydrous composition further comprises a prebiotic.

Also encompassed herein is a method for treating a mammalian subject afflicted with a disease or disorder associated with dysbiosis, the method comprising: administering a therapeutically effective amount of an anhydrous composition having the aforementioned properties (including exhibiting robustness when challenged by perturbational stress in a chemostat model test or an ecosystem output assay) to the mammalian subject, wherein the therapeutically effective amount improves relative ratios of microorganisms in the mammalian subject, thereby treating the mammalian subject. In an further embodiment, the disease or disorder associated with dysbiosis is Clostridium difficile (Clostridioides difficile) infection, Crohn's disease, irritable bowel syndrome (IBS) or spastic colon, idiopathic ulcerative colitis, mucous colitis, collagenous colitis, inflammatory bowel disease in general, microscopic colitis, antibiotic-associated colitis, idiopathic or simple constipation, diverticular disease, or AIDS enteropathy.

Also encompassed herein is an anhydrous composition comprising a co-selected microbiota for use in treating a disease or disorder associated with dysbiosis, wherein the co-selected microbiota comprises a plurality of bacterial species consisting of each of the bacterial species listed in Table 1, and optionally, at least one additional bacterial species, wherein the bacterial species listed in Table 1 are in powder-form, wherein the powder-form has a moisture content of less than 5% wt/wt in the anhydrous composition, and wherein the co-selected microbiota exhibits resistance to perturbational stress. In another aspect, an anhydrous composition comprising a co-selected microbiota for use in treating a disease or disorder associated with dysbiosis is described, wherein the co-selected microbiota comprises at least one of the bacterial species listed in Table 1, wherein the co-selected microbiota consists of bacterial species recited in Table 1, and optionally, at least one additional bacterial species, wherein the bacterial species listed in Table 1 are in powder-form, wherein the powder-form has a moisture content of less than 5% wt/wt in the anhydrous composition, and wherein the co-selected microbiota exhibits resistance to perturbational stress. In a particular embodiment, the disease or disorder associated with dysbiosis is Clostridium difficile (Clostridioides difficile) infection, Crohn's disease, irritable bowel syndrome (IBS) or spastic colon, idiopathic ulcerative colitis, mucous colitis, collagenous colitis, inflammatory bowel disease in general, microscopic colitis, antibiotic-associated colitis, idiopathic or simple constipation, diverticular disease, or AIDS enteropathy. More particularly, the bacterial species are in a state of suspended animation. In a more particular embodiment, the anhydrous composition further comprises a pharmaceutically acceptable carrier (e.g., cellulose). In a more particular embodiment, the anhydrous composition is encapsulated in a capsule (e.g., in a double capsule). In another embodiment, the at least one additional bacterial species is a species in the Acidaminococcus genus (e.g., Acidaminococcus intestini or Acidaminococcus fermentans). In another embodiment, the anhydrous composition further comprises a prebiotic.

Also encompassed herein is an anhydrous composition comprising a co-selected microbiota for use in the preparation of a medicament for treating a disease or disorder associated with dysbiosis, wherein the co-selected microbiota comprises a plurality of bacterial species consisting of each of the bacterial species listed in Table 1, and optionally, at least one additional bacterial species, wherein the bacterial species listed in Table 1 are in powder-form, wherein the powder-form has a moisture content of less than 5% wt/wt in the anhydrous composition, and wherein the co-selected microbiota exhibits resistance to perturbational stress. In another aspect, an anhydrous composition comprising a co-selected microbiota for use in the preparation of a medicament for treating a disease or disorder associated with dysbiosis is presented, wherein the co-selected microbiota comprises at least one of the bacterial species listed in Table 1, wherein the co-selected microbiota consists of bacterial species recited in Table 1, and optionally, at least one additional bacterial species, wherein the bacterial species listed in Table 1 are in powder-form, wherein the powder-form has a moisture content of less than 5% wt/wt in the anhydrous composition, and wherein the co-selected microbiota exhibits resistance to perturbational stress.

More particularly, the bacterial species are in a state of suspended animation. In a more particular embodiment, the medicament/anhydrous composition further comprises a pharmaceutically acceptable carrier (e.g., cellulose). In a more particular embodiment, the medicament is encapsulated in a capsule (e.g., in a double capsule). In another embodiment, the at least one additional bacterial species is a species in the Acidaminococcus genus (e.g., Acidaminococcus intestini or Acidaminococcus fermentans). In another embodiment, the medicament further comprises a prebiotic. In a particular embodiment, the disease or disorder associated with dysbiosis is Clostridium difficile (Clostridioides difficile) infection, Crohn's disease, irritable bowel syndrome (IBS) or spastic colon, idiopathic ulcerative colitis, mucous colitis, collagenous colitis, inflammatory bowel disease in general, microscopic colitis, antibiotic-associated colitis, idiopathic or simple constipation, diverticular disease, or AIDS enteropathy.

Other objects, features and advantages of the present invention will become clear from the following description and examples.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A and 1B show a single-stage chemostat vessel employed in the methods according to some embodiments of the present invention.

FIG. 2 depicts a chemostat model test according to one embodiment of the present invention.

FIG. 3 shows a histogram of relative percent composition of bacterial species within each of the indicated phyla according to one embodiment of the present invention.

FIGS. 4A and 4B each show a bar graph of relative percent composition of bacterial species within each of the indicated families according to one embodiment of the present invention.

FIG. 5 (Table 2) lists the MET-2 strains with their accompanying 16S rRNA sequence fragments, designated herein SEQ ID NOs: 41-80 in order of appearance in Table 2.

FIG. 6 (Table 4) lists properties of bacterial strains in MET-2.

FIG. 7 (Table 5) lists properties of bacterial strains in MET-2, MET-2A, and MET-2B.

### DETAILED DESCRIPTION OF THE INVENTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “OTU” refers to an operational taxonomic unit, defining a species, or a group of species via similarities in nucleic acid sequences, including, but not limited to 16S rRNA gene sequences.

The term “dysbiosis” as used herein refers to an imbalance of the microbial community inhabiting a subject or inhabiting a particular tissue in a subject. The term typically refers to a decrease in beneficial microbes relative to deleterious microbes or a change in the ratio of microbes such that microbes that are normally only present in small numbers proliferate to a degree whereby they are present at elevated numbers.

As used herein, the term “state of suspended animation” as used herein with respect to a population of bacteria refers to a population of bacteria that is metabolically quiescent, but capable of resuming normal metabolic activity and proliferating in response to suitable growth promoting conditions.

The term “prebiotic” as used herein refers to “a selectively fermented ingredient that allows specific changes, both in the composition and/or activity in the gastrointestinal microflora that confers benefits upon host well-being and health”. See Roberfroid (2007, J Nutri 137:8305-8375. Particular prebiotics may be chosen for optimal results when used in conjunction with compositions described herein based on the mode of administration to the subject and the target tissue/s needing treatment. Particular prebiotics used in conjunction with compositions described herein may be food grade. Particular prebiotics envisioned for use in combination with compositions described herein include: inulin, fructo-oligosaccharides, or gluco-oligosaccharides and mixtures thereof.

Solutions of bacterial species are freeze dried/lyophilized to generate anhydrous compositions comprising a plurality of bacterial species having a moisture content of less than 25%, 20%, 15%, 10% 9%, 8%, 7%, 6%, 5%, 4%, 3%, 2%, or 1%. In a particular embodiment, anhydrous compositions described herein are freeze dried to a moisture content of less than 5%.

As used herein, the term “freeze dried/lyophilized” refers to a laboratory method where live microbes in aqueous suspension are rapidly frozen to <50° C., and then the majority of the frozen water content is forced to sublime under vacuum conditions, allowing this water to be efficiently removed in the gaseous phase.

As used herein, the term “anhydrous composition comprising a plurality of bacterial species” refers to a manmade, freeze dried/lyophilized population of bacterial species having a moisture content of less than 25%, 20%, 15%, 10% 9%, 8%, 7%, 6%, 5%, 4%, 3%, 2%, or 1%. In one embodiment, a plurality of bacterial species is isolated from the fecal matter of a single, healthy individual wherein the plurality of bacterial species have been co-selected and co-adapted as an interactive population.

Technologies typically used for moisture determination include, without limitation: thermogravimetric analysis (oven drying, halogen/IR drying, microwave drying, etc.); chemical analysis (Karl Fischer titration, calcium carbide testing); spectroscopic analysis (IR spectroscopy, microwave spectroscopy, proton nuclear magnetic resonance spectroscopy); and other analyses (e.g. gas chromatography, density determination, refractometry, etc.). With respect to thermogravimetric analysis (TGA), for example, moisture content is derived from the loss of product weight during drying by measuring the change in mass of a sample while being heated at a controlled rate until no more change in weight is observed.

As used herein, the term “co-selected microbiota” refers to a plurality of bacterial species that has collectively undergone co-selection and co-adaptation in a single subject (e.g., a healthy subject). In a particular embodiment, the co-selected microbiota has collectively undergone co-selection and co-adaptation in the intestines of a single, healthy subject. In contrast, bacterial species isolated or derived from different sources (e.g., different subjects and/or cell depositories) and combined with each other have not undergone co-selection and co-adaptation in a single subject (e.g., a healthy subject). Thus, even when combined in vitro, a plurality of bacterial species that have been isolated from different sources cannot constitute a co-selected microbiota.

As used herein, the term “subject” or “patient” is preferably an animal, including but not limited to animals such as mice, rats, cows, pigs, horses, chickens, cats, dogs, etc., and is preferably a mammal, more preferably a primate, and most preferably a human.

As used herein, the term “treating” or “treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term “preventing” or “prevention” refers to a reduction in risk of acquiring or developing a disease or disorder (i.e., causing at least one of the clinical symptoms of the disease not to develop in a subject that may be exposed to a disease-causing agent, or predisposed to the disease in advance of disease onset).

As used herein, the term “prophylaxis” is related to “prevention” and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

As used herein, the phrase “pharmaceutically acceptable” refers to molecular entities and compositions that are physiologically tolerable and do not typically produce an allergic or similar untoward reaction, such as gastric upset, dizziness and the like, when administered to a human.

As used herein, the phrase “therapeutically effective amount” is used to refer to an amount of an agent (e.g., a therapeutic agent) sufficient to reduce a pathological feature of a disease or condition by at least about 30 percent, by at least 50 percent, or by at least 90 percent. A “therapeutically effective amount” of an agent results in a clinically significant reduction in at least one pathological feature (e.g., a clinical symptom) of a disease or condition.

As used herein, the term “complementary” refers to two DNA strands that exhibit substantial normal base pairing characteristics. Complementary DNA may, however, contain one or more mismatches.

As used herein, the term “hybridization” refers to the hydrogen bonding that occurs between two complementary DNA strands.

As used herein, the term “nucleic acid” or a “nucleic acid molecule” refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

As used herein, the terms “natural allelic variants”, “mutants”, and “derivatives” of particular sequences of nucleic acids refer to nucleic acid sequences that are closely related to a particular sequence but which may possess, either naturally or by design, changes in sequence or structure. By closely related, it is meant that at least about 60%, but often, more than 85%, of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

As used herein, the terms “percent similarity”, “percent identity” and “percent homology” when referring to a particular sequence are used as set forth in the University of Wisconsin GCG software program and are known in the art.

As used herein, the phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the term “operably linked” refers to a regulatory sequence capable of mediating the expression of a coding sequence and which are placed in a DNA molecule (e.g., an expression vector) in an appropriate position relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

As used herein, the term “oligonucleotide” refers to primers and probes described herein, which are defined as a nucleic acid molecule comprised of two or more ribo- or deoxyribonucleotides, preferably more than three. The exact size of the oligonucleotide will depend on various factors and on the particular application and use of the oligonucleotide.

As used herein, the term “probe” refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

As used herein, the term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

As used herein, the term “primer” refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal to the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers and/or probes may be labeled fluorescently with 6-carboxyfluorescein (6-FAM). Alternatively primers may be labeled with 4, 7, 2′, 7′-Tetrachloro-6-carboxyfluorescein (TET). Other alternative DNA labeling methods are known in the art and are contemplated to be within the scope of the invention.

In a particular embodiment, oligonucleotides according to the present invention that hybridize to nucleic acid sequences identified as specific for one of the bacterial species and/or strains described herein, are at least about 10 nucleotides in length, more particularly at least 15 nucleotides in length, more particularly at least about 20 nucleotides in length. Further to the above, fragments of nucleic acid sequences identified as specific for one of the bacterial species and/or strains described herein represent aspects of the present invention. Such fragments and oligonucleotides specific for same may be used as primers or probes for determining the amount of the particular bacterial species and/or strain in a bacterial sample generated in vitro or in a biological sample obtained from a subject, wherein the particular species or strain may be identified by the presence of any one of SEQ ID NOs: 1-40. Primers such as those described herein (e.g., SEQ ID NOs: 81 and 82) may, moreover, be used in polymerase chain reaction (PCR) assays in methods directed to determining the amount of a particular bacterial species and/or strain in a bacterial sample generated in vitro or in a biological sample obtained from a subject, wherein the particular bacterial species and/or strain comprises any one of, for example, SEQ ID NOs: 1-40.

Further to the above, a given strain's 16S rRNA sequence is species specific and in many cases, depending on the species, strain specific as well. Further to this point, some bacterial species are highly conserved and thus, different strains may have extremely similar or even identical sequences. Most species, however, include strains wherein sequence differences are detected.

Preparation of Bacterial Samples (for 16S rRNA Sanger Sequencing)

Master Mix contents (per reaction or sample):
HPLC grade ddH2O (Caledon Laboratory Chemicals)—38.5 μL
dNTPs, working stock (Invitrogen)—3 μL
10× ThermoPol Reaction Buffer (NEB)—5 μL
V3kl/V6r primers (IDT)—1 μL of each
Taq DNA Polymerase High Purity (BioBasic)—0.5 μL
DNA Template—1 μL or 1 colony

PCR, Sequencing of Products, and Analysis of PCR Products
1) Determine how much of each Master Mix component is required by multiplying each by the number of samples. Add three additional reactions to account for pipetting error.
2) Bring the required amounts of ddH2O, dNTPs (stored at −20° C.), buffer (stored at −20° C.), primers (stored at −20° C.), 2 mL flip-cap tubes (sterile, from Axygen) and a V-bottom 96-well plate (sterile, from Fisher) into the Labconco Purifier Biological Safety Cabinet (Labconco, 08018496A). UV the hood and supplies for 15 minutes.
3) Turn the UV light off. Bring Taq (stored at −20° C.) and DNA template samples (if they are in liquid form) into the safety cabinet.
4) Prepare Master Mix in a 2 mL flip-cap tube, aliquoting all of the required reagents into the same tube. Mix it by gently inverting the tube several times.
5) Aliquot Master Mix into the 96-well plate, 48 μL per reaction (or sample).
6) If your DNA is in liquid or broth form, skip Steps 9) and 10). If your DNA is taken directly from colonies on media plates skip Step 7).
7) Add 1 μL of your DNA template per aliquoted reaction (i.e. one PCR reaction for each of your DNA template samples). For example, 27 DNA template samples (or 27 strains to be tested)=27 PCR reactions.
8) Remove the aliquoted Master Mix in the 96-well plate from the biological safety cabinet.
9) Transfer the aliquoted Master Mix in the 96-well plate into the Whitley anaerobic chamber workstation.
10) Use a sterile wooden applicator (Puritan) to touch a colony of interest and, using a twisting motion, deposit the colony into one well of the 96-well plate that contains an aliquot of Master Mix. Repeat for all bacterial strains of interest.
11) Run PCR reactions in the 96-well plate in the Eppendorf Mastercycler epgradient (Eppendorf, 5340 014805):
12) Using, for example, a Eppendorf Mastercycler (a.k.a. thermocycler) run the following:
Cycle parameters are 94° C. for (the initial) 10 minutes, (94° C. for 30s, 60° C. for 30s, 72° C. for 30s) for 30 cycles, then 72° C. for 5 minutes, and 4° C. for indefinite time.
13) Sequencing is performed via Sanger sequencing methods, which are a matter of routine practice in research-based laboratories.
14) Sequences (16S rRNA full-length rRNA sequences associated with each bacterial strain) generated are compared to databases of known sequences such as, for example, those maintained by U.S. government agencies, which can be accessed via the web (e.g., blast.ncbi.nlm.nih.gov/Blast.cgi) using known programs (e.g., BLAST).
15) When using, for example, a BLAST program and an alignment application thereof, a value of 99% or higher indicates that the template sequence and the query sequence are identical. If the template sequence and the query sequence are identical this indicates that the query sequence (which was obtained from a bacterial strain of interest) is the same identity as that which is associated with the template sequence.

TABLE 1
presents a list of MET 2 strains, which is an exemplary list of bacterial
species that exhibits robustness in chemostat model test assays
described herein if the bacterial species are derived from a co-selected
microbiota. In a particular embodiment, an exemplary list of bacterial
species that exhibits robustness in chemostat model test assays
described herein comprises at least one of the following strains listed
in Table 1, but does not exceed including each and every one of the
species recited in the exemplary list of Table 1. In a more particular
embodiment thereof, the exemplary list of bacterial species that
exhibits robustness in chemostat model test assays described herein
consists of each of the strains listed in Table 1.

| Strain name | Closest species match |
|---|---|
| NB2A-14-DS | Lachnoclostridium pacaense |
| NB2B-20-DS | [Clostridium] hathewayi |
| NB2B-20-GAM | [Clostridium] lactatifermentans |
| NB2B-13-CNA | Hespellia porcina |
| NB2A-7-D5 | [Clostridium] scindens |
| NB2B-10-NB | [Clostridium] saccharogumia |
| NB2B-6-CNA | [Eubacterium] eligens |
| NB2B-13-BHI | [Eubacterium] hallii |
| NB2A-9-NA | [Ruminococcus] obeum |
| NB2A-14-FMU | [Ruminococcus] torques |
| 14 LG | Acidaminococcus intestini |
| NB2A-8-WC | Akkermansia muciniphila |
| NB2B-9-DCM | Anaerostipes hadrus |
| NB2A-15-BHI | Anaerovorax odorimutans |
| NB2B-14-D5 | Bacteroides eggerthii |
| NB2A-12-BBE | Bacteroides ovatus |
| NB2A-15-DCM | Bacteroides timonensis |
| NB2B-3-WC | Barnesiella intestinihominis |
| NB2B-16-TSAB | Bifidobacterium adolescentis |
| NB2B-11-FAA | Bifidobacterium longum/breve |
| NB2B-9-FAA | Blautia wexlerae |
| NB2A-5-TSAB | Blautia schinkii |
| NB2B-13-DCM | Collinsella aerofaciens |
| NB2A-13-NA | Coprococcus catus |
| NB2A-2-FAA | Coprococcus comes |
| NB2B-15-DCM | Dorea formicigenerans |
| NB2A-3-NA | Dorea longicatena |
| NB2B-BHI-1 | Escherichia coli |
| NB2B-10-MRS | Agathobaculum desmolans |
| NB2A-17-FMU | [Eubacterium] rectale |
| NB2B-19-DCM | Faecalibacterium prausnitzii |
| NB2A-20-GAM | Flavonifractor plautii |
| NB2B-AER-MRS-02 | Lactobacillus paracasei |
| NB2B-16-D5 | Neglecta timonensis |
| NB2A-10-MRS | Parabacteroides distasonis |
| NB2A-29-D6 | Parabacteroides merdae |
| NB2A-12-FMU | Phascolarctobacterium succinatutens |
| NB2B-10-FAA | Roseburia intestinalis |
| NB2B-26-FMU | Roseburia inulinivorans |
| NB2B-17-NB | Ruminococcus lactaris |

In a particular embodiment, an exemplary list of bacterial species that exhibits robustness in chemostat model test assays described herein comprises at least one of the following strains listed in Table 1: NB2B-6-CNA, NB2A-9-NA, NB2A-14-FMU, NB2A-8-WC, NB2A-12-BBE, NB2B-16-TSAB, NB2B-11-FAA, NB2B-13-DCM, NB2A-2-FAA, NB2A-3-NA, NB2B-BHI-1, NB2A-17-FMU, NB2B-19-DCM, NB2B-AER-MRS-02, or NB2A-10-MRS, but does not exceed further including each and every one of the species recited in this exemplary list. In a particular embodiment thereof, the exemplary list of bacterial species that exhibits robustness in chemostat model test assays described herein comprises the following strains listed in Table 1: NB2B-6-CNA, NB2A-9-NA, NB2A-14-FMU, NB2A-8-WC, NB2A-12-BBE, NB2B-16-TSAB, NB2B-11-FAA, NB2B-13-DCM, NB2A-2-FAA, NB2A-3-NA, NB2B-BHI-1, NB2A-17-FMU, NB2B-19-DCM, NB2B-AER-MRS-02, and NB2A-10-MRS, but does not exceed further including each and every one of the species recited in the exemplary list of Table 1. In a more particular embodiment thereof, the exemplary list of bacterial species that exhibits robustness in chemostat model test assays described herein consists of the following strains listed in Table 1: NB2B-6-CNA, NB2A-9-NA, NB2A-14-FMU, NB2A-8-WC, NB2A-12-BBE, NB2B-16-TSAB, NB2B-11-FAA, NB2B-13-DCM, NB2A-2-FAA, NB2A-3-NA, NB2B-BHI-1, NB2A-17-FMU, NB2B-19-DCM, NB2B-AER-MRS-02, and NB2A-10-MRS.

In a further embodiment, the exemplary list of bacterial species that exhibits robustness in chemostat model test assays described herein comprises at least one of the following strains listed in Table 1: NB2B-20-GAM, NB2B-6-CNA, NB2A-9-NA, 14 LG, NB2A-8-WC, NB2A-12-BBE, NB2A-3-NA, NB2A-17-FMU, NB2B-19-DCM, NB2B-10-FAA, NB2B-26-FMU, but does not exceed further including each and every one of the species recited in this exemplary list. In another embodiment, the exemplary list of bacterial species that exhibits robustness in chemostat model test assays described herein comprises at least one of the following strains listed in Table 1: NB2B-20-GAM, NB2B-6-CNA, NB2A-9-NA, 14 LG, NB2A-8-WC, NB2A-12-BBE, NB2A-3-NA, NB2A-17-FMU, NB2B-19-DCM, NB2B-10-FAA, NB2B-26-FMU, but does not exceed further including each and every one of the species recited in the exemplary list of Table 1.

Table 3 sets forth additional exemplary microbiotic communities comprising the indicated bacterial strains. These exemplary microbiotic communities are designated herein MET-2A and MET-2B

| | Strain | | | | |
|---|---|---|---|---|---|
| Number | designation | Identity | MET-2 | MET-2A | MET-2B |
| 1 | NB2A-29-D6 | Parabacteroides merdae | + | + | + |
| 2 | NB2B-13-BHI | [Eubacterium] hallii | + | + | + |
| 3 | NB2A-10-MRS | Parabacteroides | + | | + |
| | | distasonis | | | |
| 4 | NB2A-12-FMU | Phascolarctobacterium | + | + | + |
| | | succinatutens | | | |
| 5 | NB2B-17-NB | Ruminococcus lactaris | + | + | |
| 6 | NB2B-16-D5 | Neglecta timonensis | + | | |
| 7 | NB2B-10-NB | [Clostridium] | + | + | |
| | | spiroforme | | | |
| 8 | NB2B-10-FAA | Roseburia intestinalis | + | + | |
| 9 | NB2A-8-WC | Akkermansia | + | + | + |
| | | muciniphila | | | |
| 10 | NB2A-9-NA | [Ruminococcus] obeum | + | | |
| 11 | NB2B-20-GAM | [Clostridium] | + | | |
| | | lactatifermentans | | | |
| 12 | NB2A-15-BHI | Anaerovorax | + | | + |
| | | odorimutans | | | |
| 13 | NB2A-14-FMU | [Ruminococcus] torques | + | | + |
| 14 | NB2A-17-FMU | Eubacterium rectale | + | + | + |
| 15 | NB2B-14-D5 | Bacteroides eggerthii | + | | + |
| 16 | NB2B-26-FMU | Roseburia inulinivorans | + | + | |
| 17 | NB2B-20-DS | [Clostridium] hylemonae | + | | |
| 18 | NB2B-3-WC | Barnesiella | + | | |
| | | intestinihominis | | | |
| 19 | NB2A-14-DS | [Clostridium] | + | | |
| | | aerotolerans | | | |
| 20 | NB2A-15-DCM | Bacteroides | + | | |
| | | stercorirosoris | | | |
| 21 | NB2A-20-GAM | Flavonifractor plautii | + | + | + |
| 22 | NB2A-3-NA | Dorea longicatena | + | + | + |
| 23 | NB2A-5-TSAB | Blautia stercoris | + | | |
| 24 | NB2B-11-FAA | Bifidobacterium longum | + | | + |
| 25 | NB2A-2-FAA | Coprococcus comes | + | | |
| 26 | NB2B-6-CNA | [Eubacterium] eligens | + | + | + |
| 27 | NB2B-AER- | Lactobacillus paracasei | + | + | + |
| | MRS-02 | | | | |
| 28 | NB2B-13-CNA | [Clostridium] oroticum | + | | |
| 29 | NB2B-15-DCM | Dorea formicigenerans | + | | |
| 30 | NB2B-BHI-1 | Escherichia coli | + | + | |
| 31 | NB2B-9-DCM | Anaerostipes hadrus | + | | + |
| 32 | NB2B-9-FAA | Blautia luti | + | + | + |
| 33 | NB2A-7-D5 | [Clostridium] scindens | + | + | + |
| 34 | NB2B-10-MRS | Eubacterium desmolans | + | + | + |
| 35 | NB2B-19-DCM | Faecalibacterium | + | + | + |
| | | prausnitzii | | | |
| 36 | NB2A-12-BBE | Bacteroides ovatus | + | + | |
| 37 | NB2A-13-NA | Coprococcus catus | + | + | + |
| 38 | NB2B-16-TSAB | Bifidobacterium | + | + | + |
| | | adolescentis | | | |
| 39 | NB2B-13-DCM | Collinsella aerofaciens | + | + | + |
| 40 | 14 LG | Acidaminococcus | + | + | + |
| | | intestini | | | |
| 41 | NB2A-2-DS | Alistipes shahii | | | + |
| 42 | NB2A-1-D5 | Bacteroides uniformis | | | + |
| 43 | NB2B-3-FMN | [Clostridium] leptum | | | + |
| 44 | NB2A-1- | Enterococcus hirae | | | + |
| | CNA_aer | | | | |
| 45 | NB2B-20-NB | Gemmiger formicilis | | | + |
| 46 | NB2B-23-CNA | Oscillibacter | | | + |
| | | valericigenes | | | |
| 47 | NB2A-31-NB | Pseudoflavonifractor | | | + |
| | | capillosus | | | |

In a further embodiment, the exemplary list of bacterial species that exhibits robustness in chemostat model test assays described herein comprises at least one of the MET-2A strains listed in Table 3, but does not exceed further including each and every one of the MET-2A species recited in Table 3. In another embodiment, the exemplary list of bacterial species that exhibits robustness in chemostat model test assays described herein comprises at least one of the following MET-2A strains listed in Table 3, but does not exceed further including each and every one of the species recited in the exemplary list of Table 1. In another embodiment, the exemplary list of bacterial species that exhibits robustness in chemostat model test assays described herein consists of the MET-2A strains listed in Table 3.

In a further embodiment, the exemplary list of bacterial species that exhibits robustness in chemostat model test assays described herein comprises at least one of the MET-2B strains listed in Table 3, but does not exceed further including each and every one of the MET-2B species recited in Table 3. In another embodiment, the exemplary list of bacterial species that exhibits robustness in chemostat model test assays described herein comprises at least one of the following MET-2B strains listed in Table 3, but does not exceed further including each and every one of the species recited in the exemplary list of Table 1. In another embodiment, the exemplary list of bacterial species that exhibits robustness in chemostat model test assays described herein consists of the MET-2B strains listed in Table 3.

As used herein, the term “ecosystem output assay” refers to a method whereby the composition of a microbial ecosystem may be determined from its functional output in terms of types and quantities of selected small molecule metabolites. Small molecule metabolites are known in the art and include, without limitation: organic acids (e.g., carboxylic acids and derivatives thereof), amino acids, alcohols (e.g., polyols), phenols, and fatty acids and conjugates thereof. Metabolites are typically measured in the range of millimolar concentrations. The MET-2 community, for example, exhibits a metabolic profile that comprises tartrate and urea and significantly elevated levels of glutamate, pyroglutamate, asparagine, glycolate, choline, thymine, and formate when compared to the metabolic profiles of bacterial communities isolated from different donors. See Yen et al. (2015, J Proteome Res 14:1472-1482).

As used herein, the term “microbial ecosystem” refers to a plurality of different bacterial species that have been grown together either in an in vitro assay or in a biological setting such as, for example, a subject's gut. In a particular embodiment, the subject may be a human.

As used herein, the term “chemostat model assay” refers to an assay wherein a plurality of bacterial species is seeded into a vessel compatible with bacterial proliferation, wherein the vessel is maintained under growth promoting conditions and comprises culture medium comprising growth factors suitable for promoting proliferation of the plurality of bacterial species. In an embodiment thereof, the proliferation of each of the bacterial species seeded into the vessel may be determined after a defined time period of incubation in the chemostat model assay. Such a determination may be made using techniques known in the art such as cell counting via automated or manual means and may be facilitated by cell staining using various dyes that are taken up by cells. Such dyes may be taken up differentially by live versus dead cells and thus, provide for distinguishing viable cells from dead or dying cells. The relative proliferation of each of the bacterial species seeded into the vessel may also be determined and total numbers of each bacterial species determined after a defined time period of incubation in the chemostat model assay. Accordingly, the chemostat model assay may be used to determine proliferation and/or proliferation rate of different bacterial species in the plurality of bacterial species seeded into the vessel and thus, provide an assay for comparing proliferation and/or proliferation rate among the different bacterial species seeded into the vessel under various growth promoting conditions.

In a particular embodiment, the number of bacterial cells may be determined using a LIVE/DEAD™ BacLight™ Bacterial Viability Kit in accordance with the manufacturer's protocol. Live versus dead cells are distinguished using the LIVE/DEAD™ BacLight™ Bacterial Viability Kit, which differentially stains dead and dying cells with compromised membranes red and live cells having intact membranes green. The differential staining facilitates an accurate assessment of viable cells in a given sample.

In a more particular embodiment, the number of cells is determined via flow cytometry used in conjunction with a LIVE/DEAD™ BacLight™ Bacterial Viability Kit, which combination facilitates measuring the different colors of the differentially stained cells via fluorescence detection in a plate reader. Such an approach reveals information as to relative values of live and dead cells in a sample and generally improves accuracy of cell counting.

The chemostat model assay, therefore, provides an assay wherein the growth of the plurality of bacterial species initially seeded into a vessel (bacterial seed population) may be determined at different defined time periods of incubation in the chemostat model assay. Using the chemostat model assay, multiple vessels can be seeded with different bacterial seed populations and the growth of the different bacterial seed populations and particular species in the different bacterial seed populations can be determined at different defined time periods of incubation. Results determined from multiple vessels run in the chemostat model assay can, in turn, be compared to determine if different bacterial seed populations respond differentially to different growth conditions and perturbational stress.

As used herein, the term “robustness” as it relates to a microbial community refers to the resistance and resilience of the community towards external perturbation/s relative to the state of the microbial community absent or prior to exposure to the external perturbation/s. Robustness may, for example, be reflected in the ability of the microbial community to maintain relative ratios of representation (numbers) of each of the different species or phylums wherein the species are classified post-perturbation as compared to pre-perturbation. Robustness may also, for example, be reflected in the ability of the microbial community to maintain metabolic output post-perturbation relative to pre-perturbation.

As used herein, the term “perturbational stress” refers to a change in at least one of substrate type, substrate availability, and xenobiotic challenge in the culturing conditions in which a population of bacterial cells is grown.

As used herein, the term “substrate” refers to a substance or compound present in the culture medium in which a population of bacterial cells is grown that is utilized metabolically by the bacterial cells.

As used herein, the term “xenobiotic challenge” refers to the introduction of a chemical substance into an ecosystem, wherein the chemical substance is not naturally produced or expected to be present within the ecosystem, or is present at a much higher concentration than in the natural situation.

Compositions described herein may be formulated for oral administration as capsules, powders, tablets, granulates, chewable foods, liquids, and beverages. In a particular embodiment, the compositions are formulated into a capsule (e.g., an enteric-coated microcapsule). In another particular embodiment, the compositions are formulated into a tablet. In yet another particular embodiment, the compositions are formulated into granulated or water soluble powders. Further particular compositions may be formulated into liquids, creams, lotions, gels dispersions or ointments for topical administration.

In a particular embodiment, a composition described herein is a powder. A powder may be administered as such or may be dissolved in a fluid, for example, for oral consumption (e.g., via capsule or double capsule) or for rectal administration via an enema. With respect to oral consumption, a powder composition may be provided in a palatable form for reconstitution as a drink or for reconstitution as a food additive. A powder composition may also be dissolved in a fluid for rectal administration via an enema (colonoscopic infusion). The powder may also be reconstituted to be infused via naso-duodenal infusion. Exemplary fluids for such purposes include physiological saline solutions.

Methods described herein are applicable to animals in general (e.g., mammals), and more particularly to humans and economically significant domestic animals, such as dogs, cats, cows, pigs, horses, sheep, mice, rats, and monkeys.

When formulated, the composition may contain further ingredients, including ingredients that confer properties relating to healthfulness, flavor, formulating, or tableting. Non-limiting examples of additional ingredients that may be incorporated in compositions described herein include: prebiotics, vitamins, minerals, nutritional supplements (e.g., fiber), sweeteners, flow aids, and fillers. When formulated for oral administration, the compositions comprise at least 0.1, 1, 5, 10, 15, 20, 25, 30, 35, 40, 45, 50, 55 or more w/w % of a composition of an anhydrous composition comprising a plurality of bacterial species described herein.

Compositions of the invention are useful in methods for treating various diseases and disorders characterized by dysbiosis. Compositions described herein may be used to promote digestive health, metabolism (nutritional heath), and weight management when administered orally or rectally. Compositions described herein may be used to treat or alleviate a positive indicator or symptom of a digestive disorder including: irritable bowel syndrome (IBS) or spastic colon, idiopathic ulcerative colitis, mucous colitis, collagenous colitis, Crohn's disease, inflammatory bowel disease in general, microscopic colitis, antibiotic-associated colitis, idiopathic or simple constipation, diverticular disease, and AIDS enteropathy.

Compositions described herein are also envisioned for use in treating or alleviating a positive indicator or symptom of a digestive disorder including: irritable bowel syndrome (IBS) or spastic colon, idiopathic ulcerative colitis, mucous colitis, collagenous colitis, Crohn's disease, inflammatory bowel disease in general, microscopic colitis, antibiotic-associated colitis, idiopathic or simple constipation, diverticular disease, and AIDS enteropathy.

Treatment regimens may be comprise administration of compositions described herein to a subject in need thereof on a daily basis (typically once or twice per day), twice or thrice weekly, bi-weekly, or once per month. Treatment regimens may also be altered as the subject's condition changes and may, furthermore, be intermittent. A suitable treatment regimen may be determined by a medical practitioner and/or may be established based on empirical results as evaluated by a medical practitioner and/or the subject being treated.

Unless stated otherwise, all percentages referred to herein are by weight based on the total weight of the composition.

### Fecal-Derived Bacterial Populations and Anhydrous Compositions Thereof

In a particular embodiment, a fecal-derived bacterial population is isolated or derived from a healthy subject.

In a particular embodiment, a fecal-derived bacterial population is derived from a subject (e.g., a healthy subject) by a method comprising:
a. obtaining a freshly voided stool sample, and placing the sample in an anaerobic chamber (in an atmosphere of 90% N₂, 5% CO₂ and 5% H₂);
b. generating a fecal slurry by macerating the stool sample in a buffer; and
c. removing food particles by centrifugation, and retaining the supernatant, which comprises the bacteria isolated from fecal matter and food particles. Accordingly, the supernatant comprises a purified population of intestinal bacteria that is free of fecal matter and food particles. Given that, the purified population of intestinal bacteria is a manmade product that is fecal matter-free and food particle-free.

In a further embodiment, a fecal sample (either fresh or frozen) is diluted in saline and plated onto a series of 13-20 different media types, each tailored to the isolation of particular types of species. The fecal sample may also be used undiluted as inoculum to seed a chemostat, which is grown to steady state, and then an aliquot of the steady state culture is diluted in saline and subsequently plated onto a series of 13-20 different media types, each tailored to the isolation of particular types of species. A diluted sample of bacteria may, for example, be treated with ethanol to select for sporulating bacteria. In another embodiment, antibiotics are added that exclude certain types of bacterial cells. In another embodiment, filter-sterile spent chemostat medium is added to provide growth substrates that promote proliferative or provide a selective advantage for certain types of bacterial cells. Following transfer into the 13-20 different media types, bacterial cell cultures are incubated for 3-10 days and individual colonies are picked, re-streaked to purity, and then frozen down. Frozen stocks are grown in culture to curate/characterize the strain by conducting a 16S rRNA gene sequencing read using Sanger chemistry and the obtained trace compared to the RDP database.

Once the strains have been curated/characterized, each bacterial species listed in Table 1 or a subset thereof is cultured individually to expand the population of each bacterial species to reach a threshold of biomass for each bacterial species. For bacterial species that grow poorly relative to other species listed in Table 1, a larger volume of bacterial culture is grown so as to achieve a biomass equivalent to that of faster growing species. The strains are all grown separately in Wilkins-Chalgren broth under anaerobic conditions at 37° C. The cultured bacterial population of each species is then concentrated by centrifugation, resuspended in medium optionally containing a cryoprotectant/lyoprotectant (inulin and riboflavin), and then rapidly frozen at −80° C. Frozen material is placed into a lyophilizer instrument and the cycle run to sublimate and remove the water content, leaving a fine powder representing a matrix of preserved bacterial cells and optionally cryo-lyoprotectant. The individual powders from each individual isolate are tested for purity and if pure, may be combined into desired combinations as powders via thorough mixing to generate an anhydrous composition comprising a desired plurality of bacterial species.

In certain embodiments, an anhydrous composition comprising a population of bacterial species may be derived from fecal matter in accordance with methods disclosed in U.S. Pat. Nos. 8,906,668 and 9,511,099 and in U.S. Patent Application Publication No. 20140342438, the entire content of each of which is incorporated herein by reference.

### Culture Methods According to Certain Embodiments

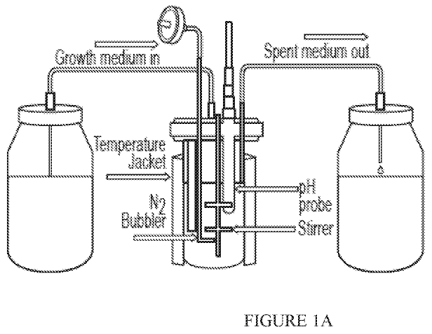

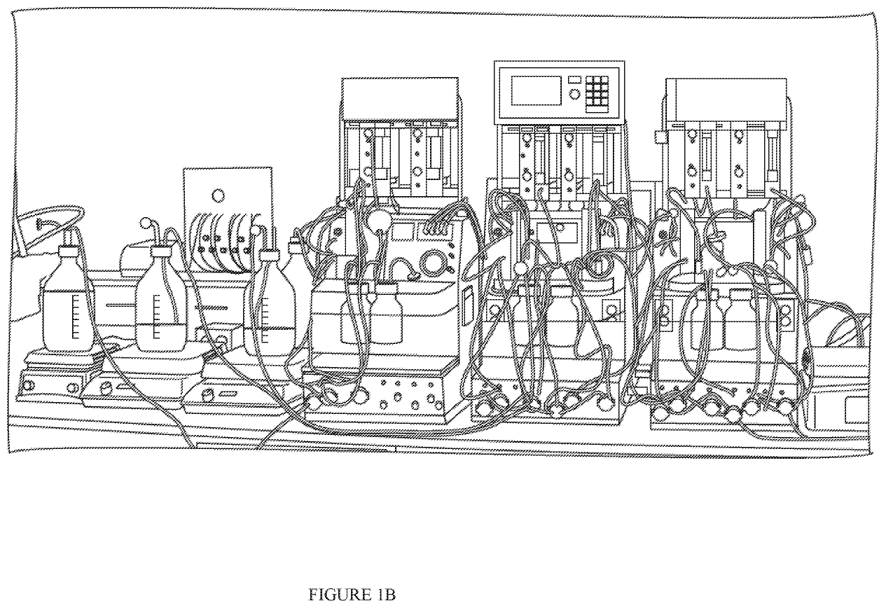

In certain embodiments, an anhydrous composition comprising a plurality of bacterial species is cultured in a chemostat vessel. In certain embodiments, the chemostat vessel is the vessel disclosed in U.S. Patent Application Publication No. 20140342438. In some embodiments, the chemostat vessel is the vessel described in FIGS. 1A and 1B.

In certain embodiments, the chemostat vessel is converted from a fermentation system to a chemostat by blocking off the condenser and bubbling nitrogen gas through the culture. In certain embodiments, the pressure forces the waste out of a metal tube (formerly a sampling tube) at a set height and allows for the maintenance of given working volume of the chemostat culture.

In certain embodiments, the chemostat vessel is kept anaerobic by bubbling filtered nitrogen gas through the chemostat vessel. In certain embodiments, temperature and pressure are automatically controlled and maintained

In certain embodiments, the culture pH of the chemostat culture is maintained using 5% (v/v) HCl (Sigma) and 5% (w/v) NaOH (Sigma).

In certain embodiments, the culture medium of the chemostat vessel is continually replaced. In certain embodiments, the replacement occurs over a period of time equal to the retention time of the distal gut. Consequently, in certain embodiments, the culture medium is continuously fed into the chemostat vessel at a rate of 400 mL/day (16.7 mL/hour) to give a retention time of 24 hours, a value set to mimic the retention time of the distal gut. An alternate retention time can be 65 hours (approximately 148 mL/day, 6.2 mL/hour). In certain embodiments, the retention time can be as short as 12 hours.

In certain embodiments, the culture medium is a culture medium disclosed in U.S. Patent Application Publication No. 20140342438.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

Reference is now made to the following examples, which together with the above descriptions illustrate some embodiments of the invention in a non limiting fashion.

### EXAMPLES

### Example 1

### Comparison of Microbial Ecosystems Derived from a Single Donor Versus Those Derived from Multiple Donors

The present inventors investigated whether microbes derived from a single individual have co-adapted to the host and demonstrate ‘cohesiveness’ or an ability to work efficiently together. The present inventors hypothesized that such cohesiveness may be critical to the ability of the ecosystem to best respond to commonly encountered environmental perturbations

To test this, the present inventors created 2 defined microbial communities of 27 bacterial species each, representing 6 bacterial phyla commonly found in the human gut. The first community (CC) represented a group of bacterial isolates, each representative of a different species, which had been isolated from a single donor. Accordingly, CC is a co-selected microbiota. The second community (FC) represented a group of isolates which matched the CC community in species identity (≥97% identity across the full length 16S rRNA gene sequences), but wherein each community member had been sourced from a different individual (i.e., 27 different individuals in total). The communities were verified for purity by individual deep sequencing of 16S rRNA genes on the Illumina Miseq platform.

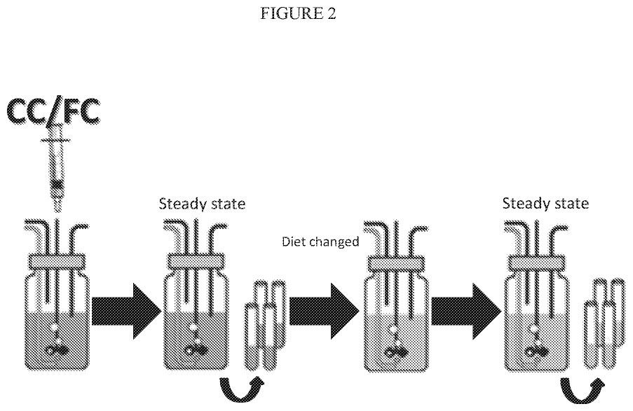

Each community was separately seeded into a bioreactor vessel fed with a high fibre diet and allowed to achieve steady state (14 days), and then samples were removed for analysis. See FIG. 2.

After sample removal at steady state, the bioreactors were switched abruptly to a high protein diet to simulate a perturbational stress. Steady state was allowed to develop over a further 14 days and the ecosystems were resampled. See FIG. 2. 16S rRNA sequence profiling was carried out on the samples using the Illumina MiSeq platform.

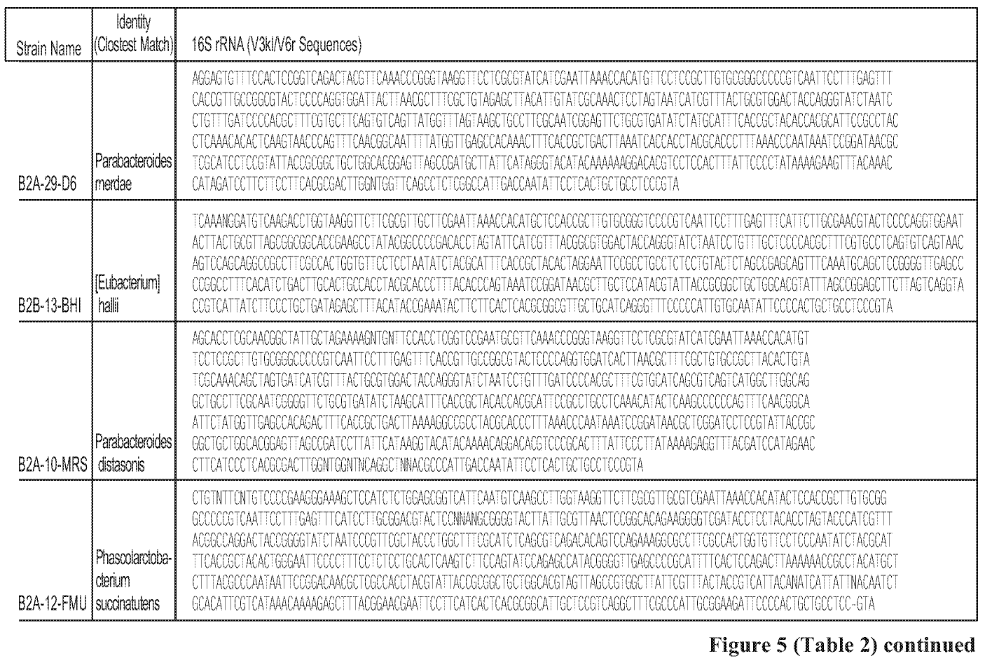

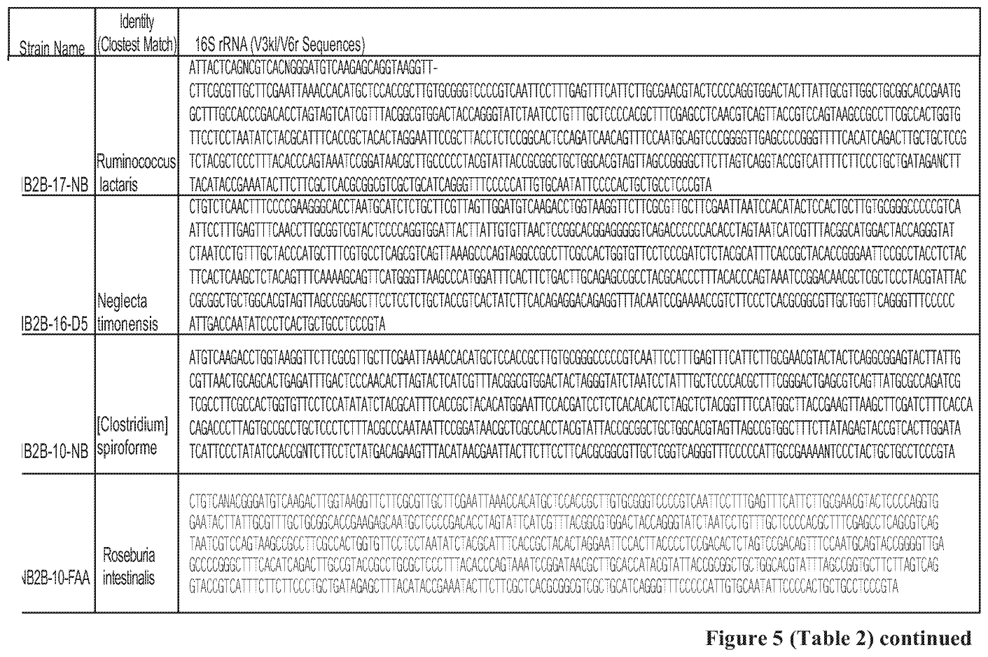

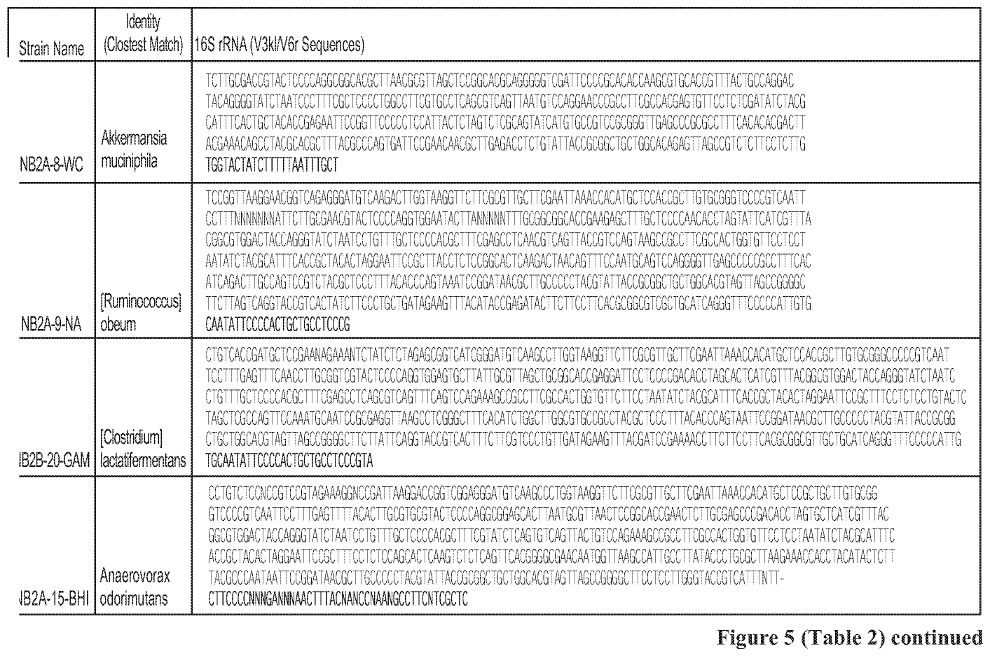

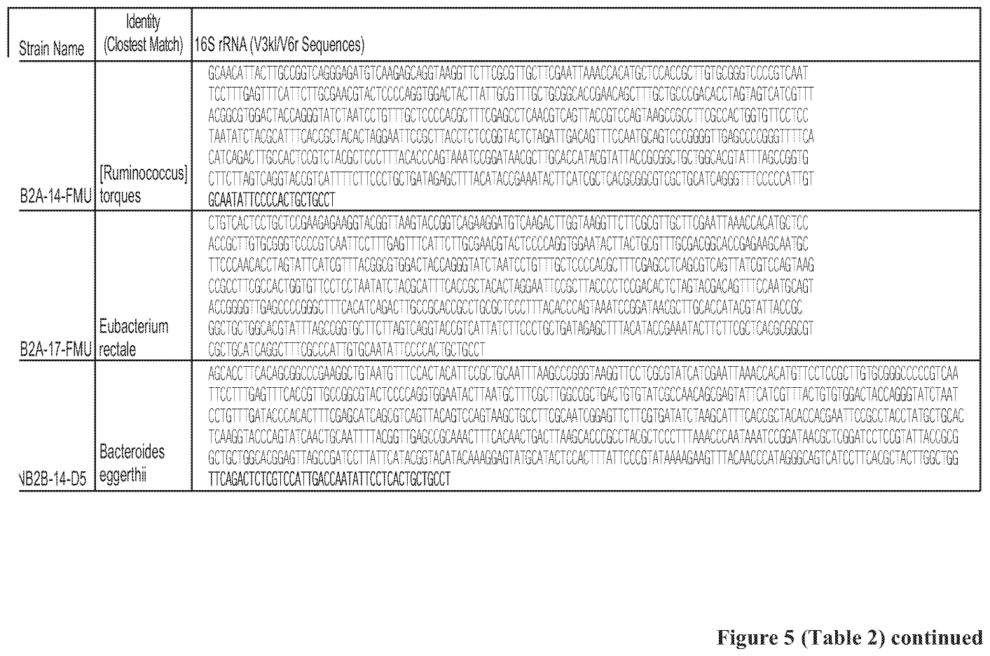

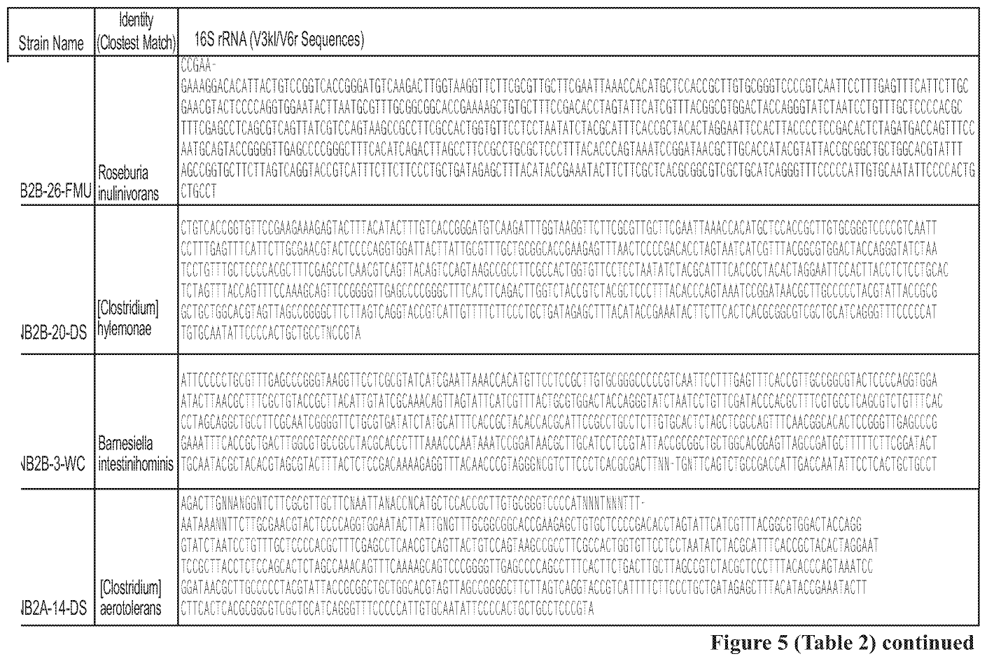

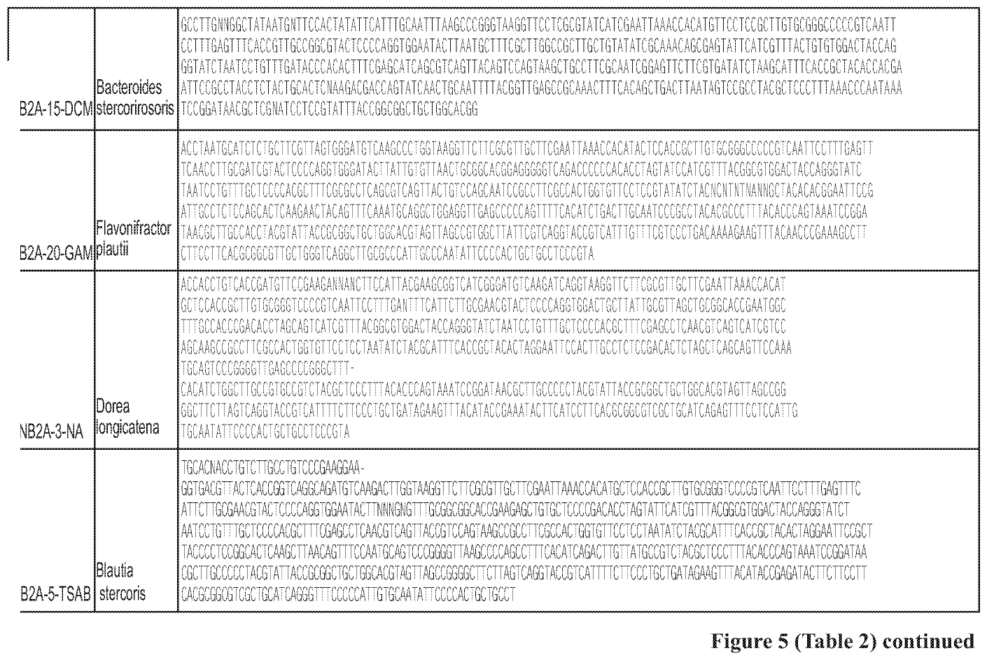

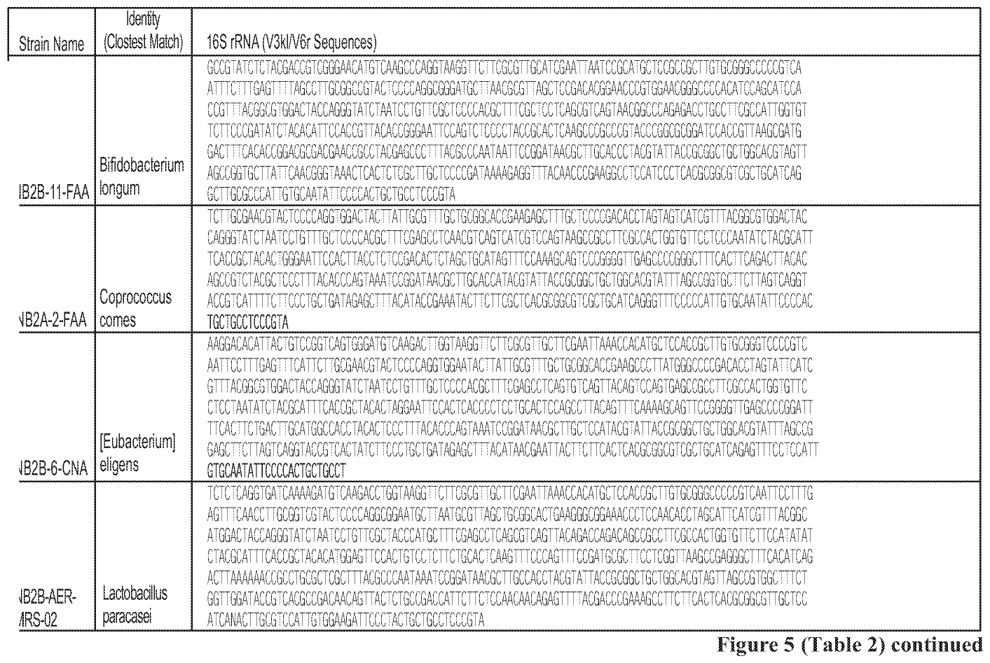

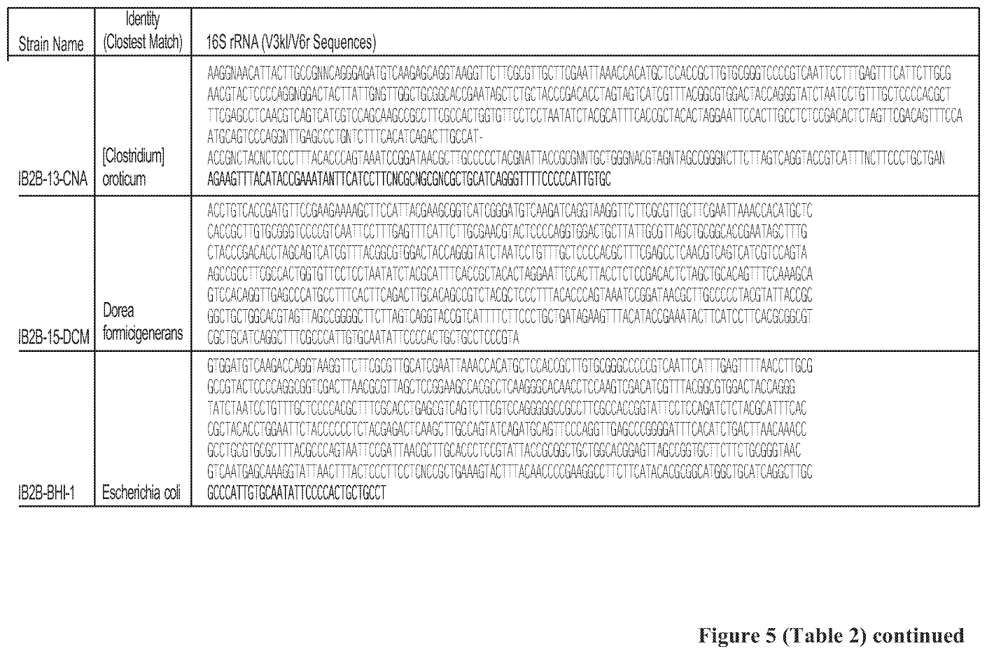

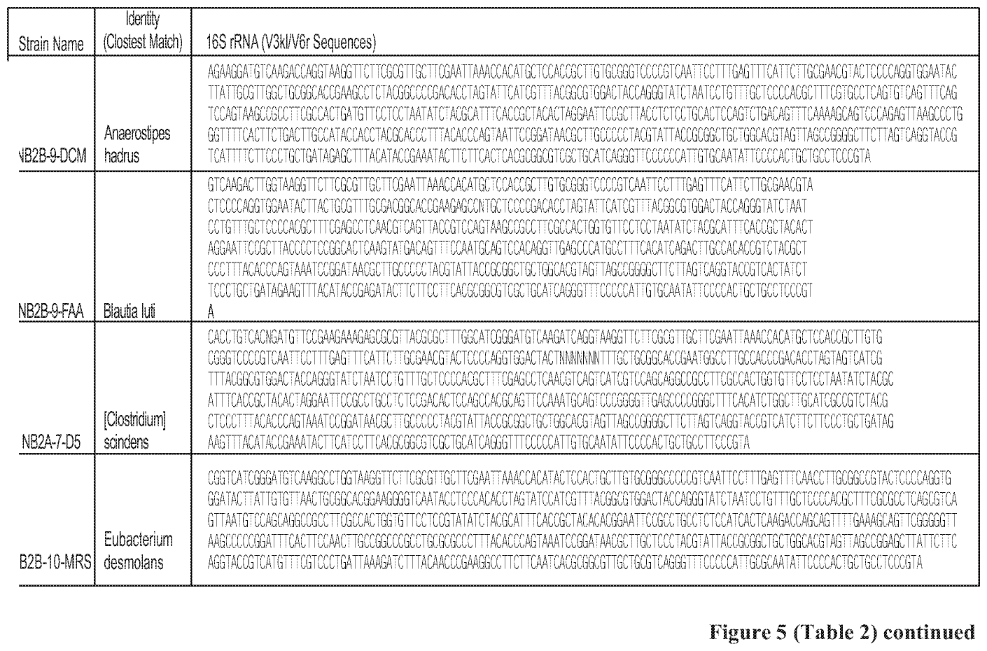

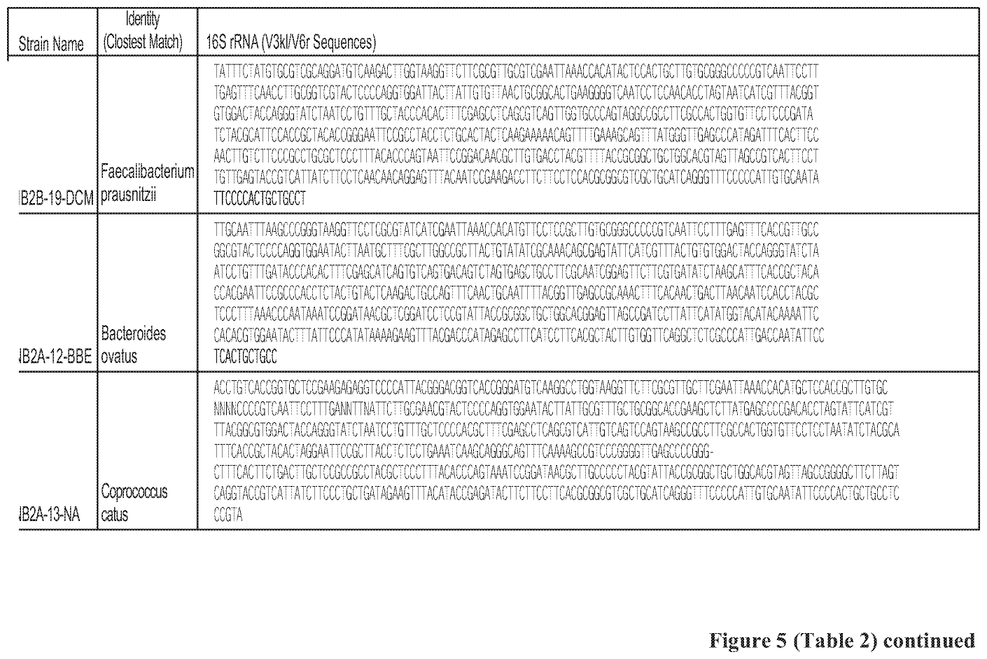

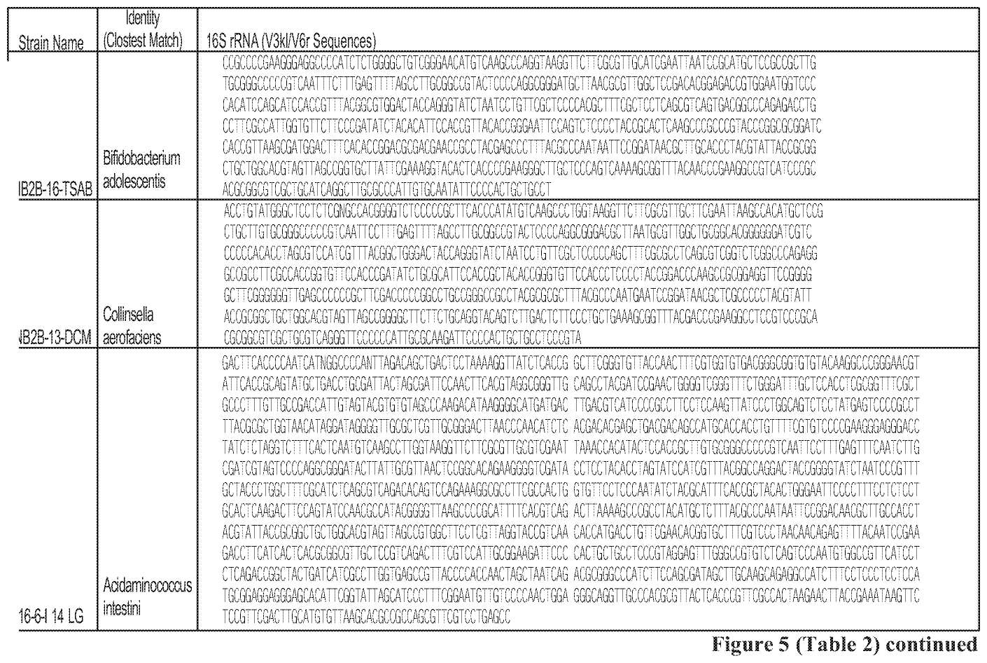

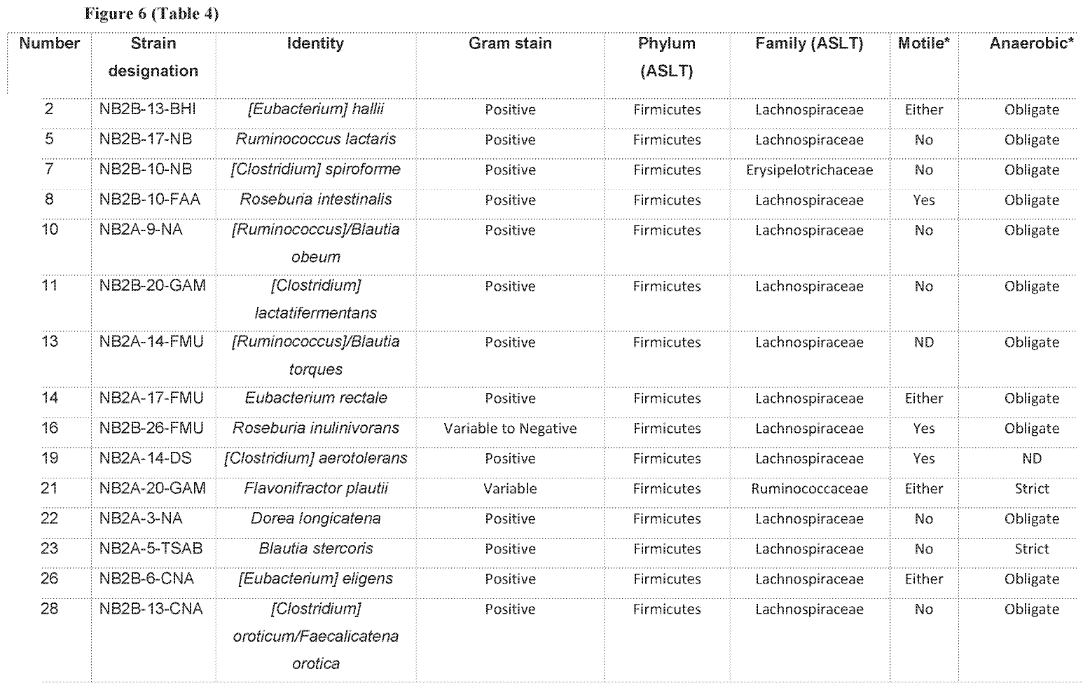

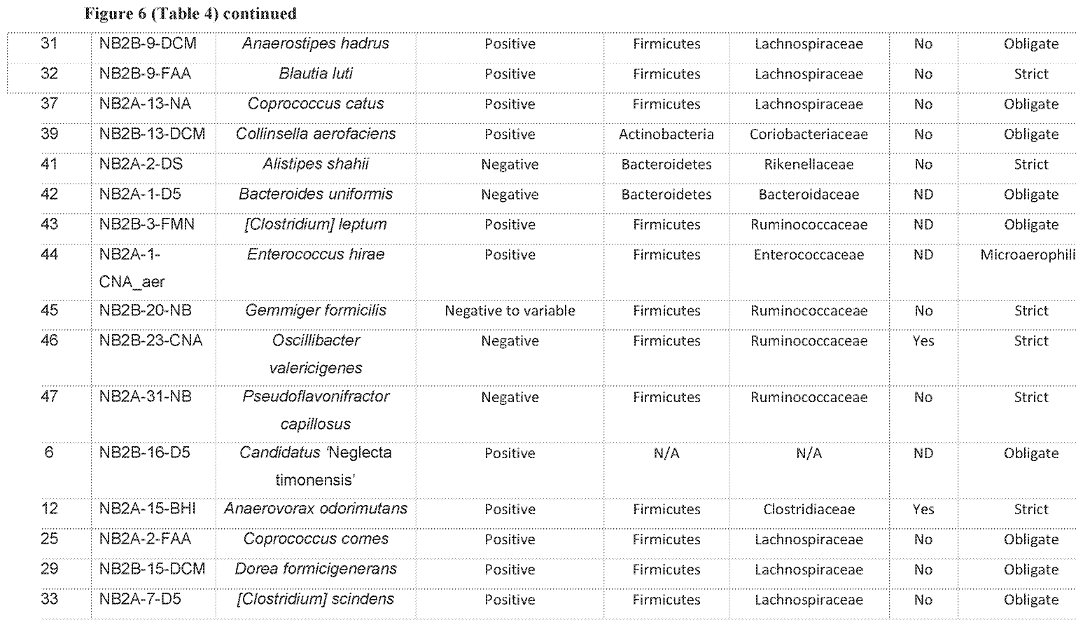

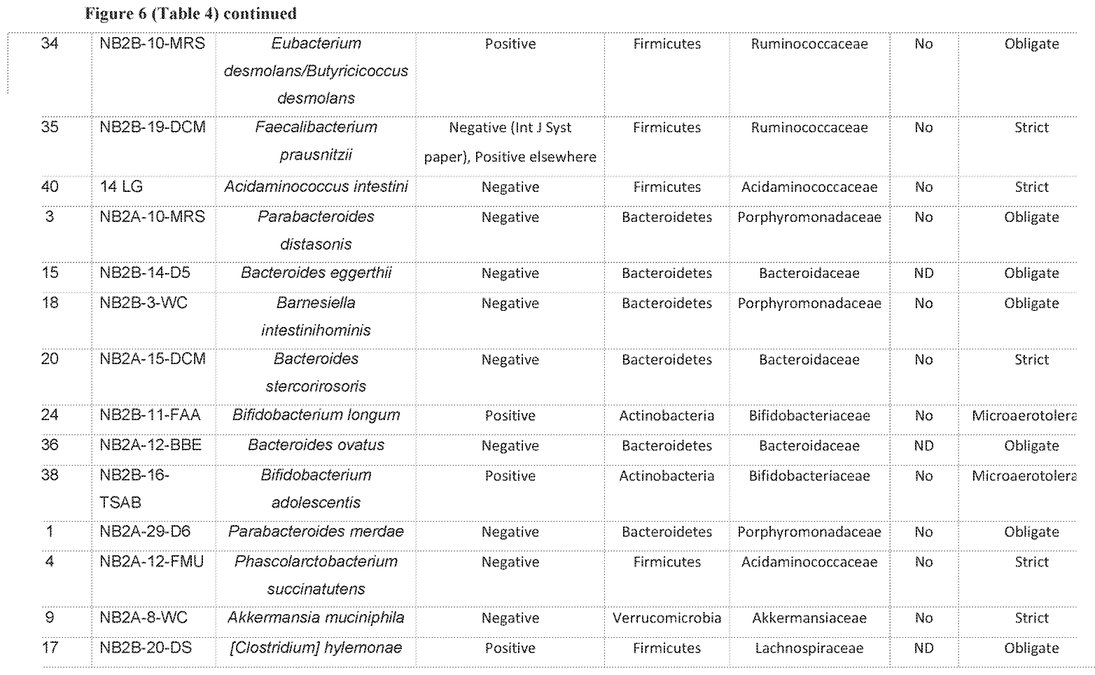

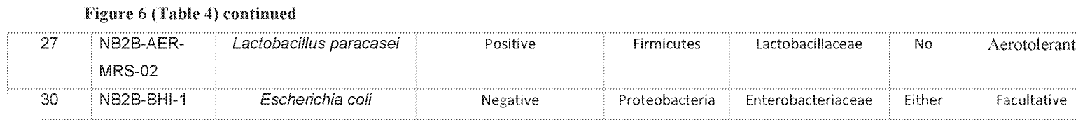

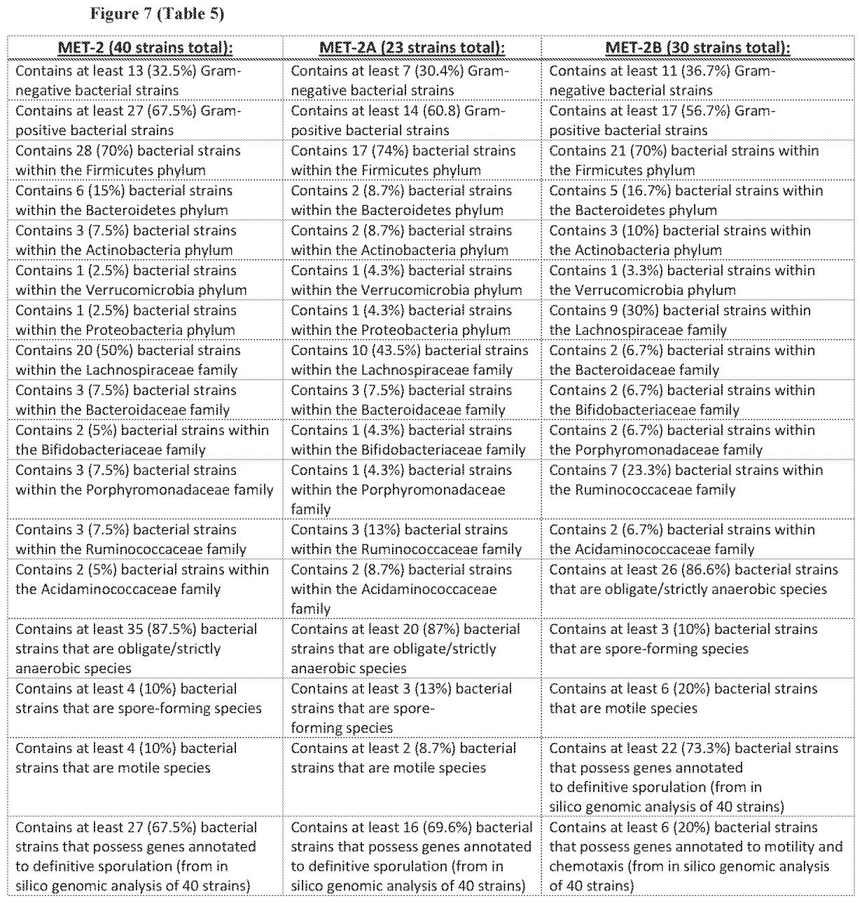

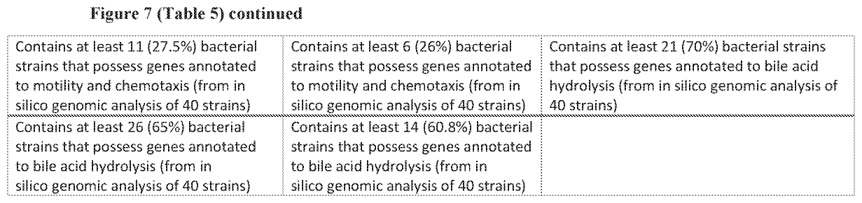

16S rRNA primers were used to generate 16S rRNA sequence fragments FIG. 5 (Table 2) and full length 16S rRNA sequences (Appendix A) corresponding to each bacterial strain presented in Table 1. Exemplary 16S rRNA primers with T3 and T7 tails, respectively are as follows:

V3kl primer
(SEQ ID NO: 81)
ATTAACCCTCACTAAAGTACGG+AG+AGGCAGCAG
V6r primer
(SEQ ID NO: 82)
AATACGACTCACTATAGGGAC+AG+ACACGAGCTGACGAC.

Full length 16S rRNA sequences for each of the MET-2 strains are presented in Appendix A (attached hereto) and designated SEQ ID NOs: 1-40.

FIG. 5 (Table 2) lists the MET-2 strains with their accompanying 16S rRNA sequence fragments. The 16S rRNA sequence fragments are designated SEQ ID NOs: 41-80 in order of their appearance in Table 2.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Results

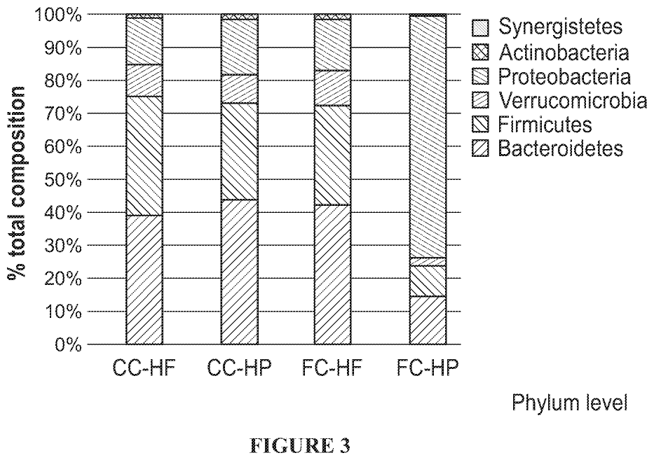

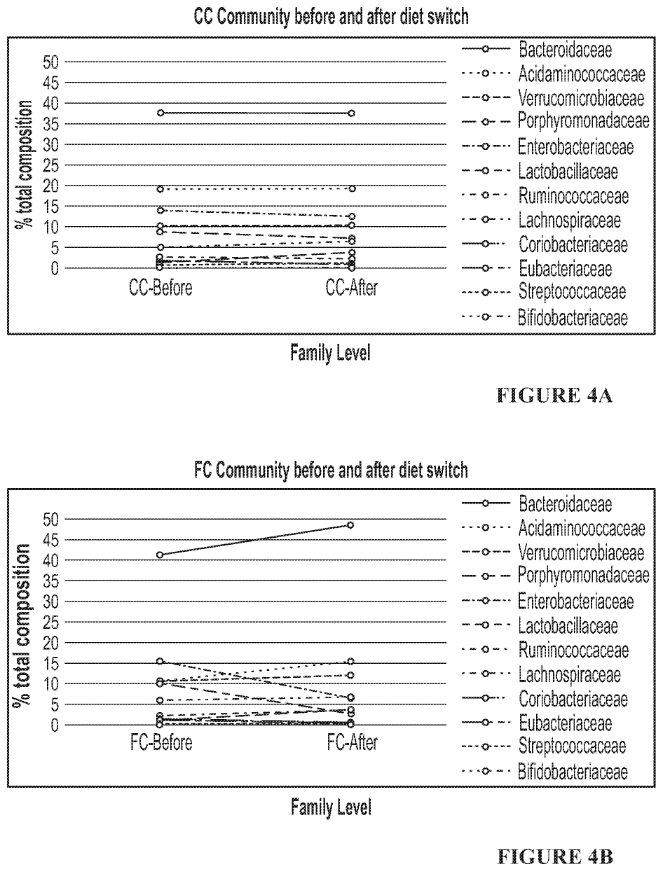

A switch from high fibre to high protein diet resulted in little appreciable change in the relative abundance profiles of the CC community, suggesting that the community could efficiently adapt to the perturbation. Conversely, the FC community showed a dramatic change in relative abundance with a marked increase in Proteobacteria—a common sign of dysbiosis. Proteobacteria include many microbial species that tend to be metabolically versatile and thus opportunistic feeders. See FIGS. 3 and 4.

Based on the results presented herein, the present inventors conclude that microbial communities that have been co-selected and co-adapted within a host (co-selected microbiota) demonstrate robustness during perturbational stress. This provides justification for creating MET products derived from a single selected donor rather than an amalgamation of many donors.

Protocol for Treating Humans Afflicted with Diseases or Disorders Associated with Dysbiosis

As described herein, MET-2 and exemplary subgroups thereof (e.g., MET-2A and MET-2B) are described as therapeutic agents for treating gastrointestinal diseases in subjects afflicted with such diseases, including ulcerative colitis. The bacterial isolates found in MET-2 are pure live bacterial cultures of intestinal bacteria that were isolated from a stool sample of a healthy 25-year-old male donor. The microbial ecosystem therapeutic product is comprised of 40 lyophilized pure bacterial cultures mixed in predefined ratios. The product is delivered to the patients orally, in capsule form.

MET-2 comprises 40 strains of lyophilized bacteria, originally purified from a healthy 25-year-old stool donor and further selected based on their favorable safety profile. The donor used to derive MET-2 was also successfully used as a donor for FMT in the treatment of multiple patients with Clostridium difficile. A phase la clinical trial with MET-2 in patients with rCDI is currently in progress. Preliminary evidence suggests MET-2 is well tolerated with no serious adverse events related to treatment with this therapeutic to date. Furthermore, there have been no reported cases of bacteremia, sepsis or invasive infections in rCDI patients undergoing MET-2 treatment to date.

MET-2 has modifications that reflect and incorporate novel information that has emerged from the rapidly evolving field of gut microbiota research in the context of ulcerative colitis. The donor from which MET-2 was derived was screened extensively for viral, bacterial and medical disease. Briefly, MET-2 excludes pathogenic organisms including extended spectrum beta-lactamase (ESBL), vancomycin-resistant Enterococcus (VRE), methicillin-resistant Staphylococcus aureus (MRSA), and Clostridium difficile (C. difficile). In addition, risk assessment for high-risk behaviors for blood-borne pathogens, a thorough detailed medical history and a physical examination to confirm the overall health of the donor were completed. There are no specific considerations for MET-2 recipients. Empiric and targeted antibiotic therapy should be guided by routine standards of care in close consultation with appropriate experts including infectious disease or medical microbiology specialists. The isolated strains were then purified by repeated subculture, initially sequenced for identification and screened for bacterial resistance to ensure no transfer of resistant strains. Within the manufacturing process, there are multiple passaging steps, where purity is subsequently examined on plate culture. Finally, MET-2 product release only occurs when each bacterial culture tests negative for impurities or any bacterial contaminant (e.g. pathogenic organisms) as determined by Sanger sequencing of the 16S rRNA gene (specific to bacteria).

MET-2 is comprised of 40 lyophilized pure bacterial cultures mixed in predefined ratios, with strengths as detailed in Table 6.

TABLE 6
Strength of MET-2 Capsules

| MET-2 Dosage | MET-2 content | MET-2 content by |
|---|---|---|
| Form | by weight (g) | Colony Forming Units |
| Capsule | 0.5 | 3.59 × 10⁷ to 3.59 × |
| | | 10¹¹ CFU per capsule |

CFU = colony forming unit

Ulcerative colitis (UC) is a chronic, relapsing, idiopathic, inflammatory disease of the colorectum. In the last decade, there has been an increase in the incidence and prevalence of UC, making it an important emerging global disease. The main symptoms of UC include bloody diarrhea, abdominal pain, urgency, tenesmus, and incontinence, which cause a reduction in patient quality of life. The severity of UC symptoms ranges from mild disease (<4 stools per day with or without blood) to severe disease (>10 stools per day with intense cramping and continuous bleeding). Depending on the clinical severity of intestinal disease, patients may also develop systemic symptoms and other life-threatening complications.

Management of UC is determined by the clinical severity of disease, and current treatment strategies are focused on regulating the immune system with anti-inflammatory and immunosuppressive drugs. For mild-to-moderate disease anti-inflammatory agents, e.g. 5-aminosalicyclic acid (5-ASA), are the main treatment options with use of immunomodulators as a steroid sparing agent. While these therapies are able to maintain remission in many cases, current medical treatments are imperfect and there is a subset of patients that do not respond to topical 5-ASA alone or in combinational therapy with corticosteroids. Additionally, 20-30% of UC patients require colectomy to manage acute complications and medically intractable disease. Thus, there is a need for more efficacious drugs with a greater favorable safety profile for the treatment of UC.

Although the pathogenesis of UC is complex, multifactorial, and not fully understood, aberrant host immune responses, and a dysfunctional intestinal barrier have been associated with this condition.

The human body is host to more than 10 trillion microbial cells with the majority of these residing in the gut. The collection of microorganisms, their gene products and corresponding metabolic functions in the human gastrointestinal (GI) tract is termed the gut microbiome. Recent advances in molecular microbiology have revealed the critical role of the gut microbiome in a variety of important processes including: vitamin/nutrient production, regulation of metabolism and host energy demands, intestinal epithelial cell homeostasis, protection against pathogens, and development and maintenance of normal immune function.

Gut dysbiosis can be defined as a pathological imbalance in a microbial community characterized by a shift in the composition, diversity or function of microbes, which can result in disease. Antibiotics, toxic compounds, diet, medical interventions, and disease can all influence the gut microbiome. However, defining gut microbial dysbiosis is difficult due to the variability in bacterial composition across individuals in both in healthy and disease-states. The gut microbiome has been associated with a multitude of disease indications including, but not limited to: C. difficile infection (CDI), inflammatory bowel disease (IBD), and irritable bowel syndrome (IBS).

The MET-2 anhydrous composition (drug product) comprises a lyophilized mixture of predetermined ratio of pure cultures of 40 diverse intestinal bacteria, derived from a stool sample of one healthy donor. Each capsule contains 0.5 g of MET-2, with a strength per capsule of 3.59 X 10⁷ to 3.59×10¹¹ colony forming units (CFU) per capsule. The drug product is shipped and maintained at room temperature; the capsule is sealed in anaerobic packaging and is opened only immediately prior to the subject/patient swallowing the capsule.

As indicated above, forty pure bacterial culture isolates have been selected for MET-2 composition from a stool sample of a single donor. The identities of the bacterial isolates have been confirmed microbiologically as well as using 16S ribosomal RNA (rRNA) sequencing. All isolates included in MET-2 are sensitive to imipenem, ceftriaxone, and piperacillin. Susceptibility to antimicrobials was determined by directly measuring susceptibility with e-strips and/or Kirby Bauer disks.

List of cultured isolates that have been selected for the drug substance are provided in Table 7 below.

TABLE 7
MET-2 Composition

| | | | CFU of strain |
|---|---|---|---|
| | | % | per 3-capsule |
| Strain ID | Identity (closest match)^{a} | Identity | dose |
| 14 LG | Acidaminococcus | 99 | 2.7 × 10⁵-10⁹ |
| | intestini | | |
| NB2A-8-WC | Akkermansia mucimphila | 100 | 4.0 × 10⁶-10¹⁰ |
| NB2B-9-DCM | Anaerostipes hadrus | 99.53 | 1.0 × 10²-10⁶ |
| NB2A-15-BHI | Anaerovorax | 95.76 | 4.0 × 10⁵-10⁹ |
| | odorimutans | | |
| NB2B-14-D5 | Bacteroides eggerthii | 99.71 | 2.4 × 10⁶-10¹⁰ |
| NB2A-12-BBE | Bacteroides ovatus | 99 | 1.3 × 10³-10⁷ |
| NB2A-15-DCM | Bacteroides | 100 | 3.5 × 10⁵-10⁹ |
| | stercorirosoris | | |
| NB2B-3-WC | Barnesiella | 99 | 2.9 × 10⁵-10⁹ |
| | intestinihominis | | |
| NB2B-16-TSAB | Bifidobacterium | 99.71 | 6.3 × 10⁵-10⁹ |
| | adolescentis | | |
| NB2B-11-FAA | Bifidobacterium longum | 99 | 1.8 × 10³-10⁷ |
| NB2B-9-FAA | Blautia luti | 99 | 3.4 × 10⁵-10⁹ |
| NB2A-5-TSAB | Blautia stercoris | 98.6 | 2.0 × 10⁵-10⁹ |
| NB2A-14-DS | [Closfridium] | 97.8 | 1.24 × 10⁵-10⁹ |
| | aerotolerans | | |
| NB2B-20-DS | [Closfridium] hylemonae | 95.73 | 5.0 × 10³-10⁷ |
| NB2B-20-GAM | [Closfridium] | 96.33 | 2.1 × 10⁵-10⁹ |
| | lactatifermentans | | |
| NB2B-13-CNA | [Closfridium] oroticum | 95.91 | 2.0 × 10⁶-10¹⁰ |
| NB2A-7-D5 | [Closfridium] scindens | 99 | 3.2 × 10⁵-10⁹ |
| NB2B-10-NB | [Closfridium] spiroforme | 97.93 | 1.3 × 10⁶-10¹⁰ |
| NB2B-13-DCM | Collinsella aerofaciens | 99.85 | 4.1 × 10⁶-10¹⁰ |
| NB2A-13-NA | Coprococcus catus | 99 | 1.9 × 10⁵-10⁹ |
| NB2A-2-FAA | Coprococcus comes | 100 | 5.6 × 10⁶-10¹⁰ |
| NB2B-15-DCM | Dorea formicigenerans | 99 | 8.0 × 10⁵-10⁹ |
| NB2A-3-NA | Dorea longicatena | 99.27 | 3.1 × 10⁴-10⁸ |
| NB2B-BHI-1 | Escherichia coli | 99 | 4.2 × 10⁷-10¹¹ |
| NB2B-10-MRS | Eubacterium desmolans | 99 | 2.5 × 10⁵-10⁹ |
| NB2A-17-FMU | Eubacterium rectale | 100 | 9.2 × 10³-10⁷ |
| NB2B-6-CNA | [Eubacterium] eligens | 99 | 3.5 × 10⁶-10¹⁰ |
| NB2B-13-BHI | [Eubacterium] hallii | 99 | 3.2 × 10²-10⁶ |
| NB2B-19-DCM | Faecalibacterium | 99 | 3.4 × 10³-10⁷ |
| | prausnitzii | | |
| NB2A-20-GAM | Flavonifractor plautii | 99 | 7.1 × 10⁵-10⁹ |
| NB2B-AER- | Lactobacillus paracasei | 99.85 | 5.9 × 10⁵-10⁹ |
| MRS-02 | | | |
| NB2B-16-D5 | Neglecta timonensis | 99.86 | 3.6 × 10³-10⁷ |
| NB2A-10-MRS | Parabacteroides | 99 | 3.5 × 10³-10⁷ |
| | distasonis | | |
| NB2A-29-D6 | Parabacteroides merdae | 99.85 | 1.7 × 10⁵-10⁹ |
| NB2A-12-FMU | Phascolarctobacterium | 99 | 2.5 × 10⁴-10⁸ |
| | succinatutens | | |
| NB2B-10-FAA | Roseburia intestinalis | 99.69 | 8.6 × 10⁴-10⁸ |
| NB2B-26-FMU | Roseburia inulinivorans | 99.4 | 1.7 × 10³-10⁷ |
| NB2B-17-NB | Ruminococcus lactaris | 99 | 1.3 × 10⁵-10⁹ |
| NB2A-9-NA | [Ruminococcus] obeum | 99 | 7.5 × 10⁴-10⁸ |
| NB2A-14-FMU | [Ruminococcus] torques | 99 | 2.2 × 10⁶-10¹⁰ |

^{a}Closest species match was inferred by alignment of the 16S rRNA sequence to the NCBI database; note that in some cases 16S rRNA gene sequences could not resolve identity beyond genus, and that closest match does not infer definitive speciation. Note that some representative strains identify with the same species by 16S rRNA gene sequence alignment but are believed to be different strains based on observed differences in colony morphology, antibiotic resistance patterns and growth rates.

Further to the above, any potential strain having equal to or greater than 97% identity to its closest neighbor by 16S rRNA gene sequence identity is considered in the art to be of the same species. This accepted understanding applies to all percent identities described herein.

Microcrystalline cellulose is added to the mixture of lyophilized drug substances as a flow aid. Two-piece hard Vcaps® Enteric Capsules (Capsugel), composed of hypromellose/hypromellose AS and titanium dioxide, are used to encapsulate the MET-2 drug substance mixture (including microcrystalline cellulose). The MET-2 product is double-encapsulated; MET-2 lyophilized material is filled into a size 0 enteric capsule, sealed, and then placed in a size 00 enteric capsule, which is then sealed again.

MET-2 capsules are administered orally in an enteric capsule, for delivery of the live bacteria to the large intestine. MET-2 capsules are to be stored at room temperature, and packaging should be opened only immediately before administration to patients in order to preserve the nitrogen atmosphere within the packages.

Preclinical Studies

Dextran sulfate sodium (DSS) is a commonly-employed mouse model of colitis which involves a chemical disruption of barrier function in the absence of involvement of any specific pathogen.

In order to explore the effects of MET-2 on barrier function and inflammation, mice may be gavaged with MET-2 following an oral antibiotic treatment and then given 3% DSS to induce colitis. Mice receiving MET-2 may be evaluated to measure serum levels of inflammatory cytokines as well as reduced histologic injury compared to controls. In addition, the effect of MET-2 administration following oral antibiotics may be measured to evaluate if MET-2 administration attenuates the DSS-mediated loss of Mucin-2, a mucin protein and major constituent of the protective mucous barrier found in the colon. Impaired gut barrier function can initiate dysbiosis which influences gut barrier integrity and innate and adaptive immune responses in the host. Maintenance of gut barrier integrity is critical in the context of gut homeostasis as inappropriate immune responses to a dysbiotic gut microbiota are hypothesized contribute to the pathogenesis of UC.

In vitro studies have already been performed with MET-2 formulations. These studies have shown that MET-2 protected human intestinal cell lines from cytoskeleton and cell barrier damage caused by C. difficile toxins C. difficile Toxin A (TcdA) and C. difficile Toxin B (TcdB). MET-2 also protected cells from apoptosis.

Results of Clinical Studies with MET-2

Preliminary evidence suggests that MET-2 is well tolerated with no serious adverse events related to treatment with this therapeutic to date. Additionally, there have been no reported cases of bacteremia, sepsis or invasive infections in rCDI patients undergoing MET-2 treatment to date.

Clinical development for MET-2 includes rigorous donor screening. To summarize, fecal material was obtained from a healthy fecal donor with informed and written consent. The donor was screened for a variety of blood borne disease such as HIV-1 and HIV-2; hepatitis A, B and C; syphilis as well as different enteric bacteria (Salmonella species, Shigella species, Campylobacter species, Escherichia coli 0157:H7 and Yersinia) and the presence of C. difficile toxins. The stool was also examined for microscopic presence of ova and parasites. The donor was further screened for colonization with Helicobacter pylori, methicillin-resistant Staphylococcus aureus and vancomycin-resistant Enterococcus species in stool. A detailed medical history, including high risk behavior and a physical examination were also completed.

Bacterial strains were purified and grown in a bioreactor modeling the conditions of the human distal gut. Susceptibility to antimicrobials was determined. Isolates representing commensal species, sensitive to a range of antimicrobials, were selected for the final stool substitute formulation. Full length 16S rRNA sequences were classified using basic local alignment search tool (BLAST) with the most specific name used to report the DNA maximum likelihood score. MET-2 constituent strains were individually grown in pure culture, snap-frozen, and subjected to lyophilization. After each strain meets CFU/g specifications, lyophilized bacterial product from all strains were combined in pre-determined ratios to make the active pharmaceutical ingredient (API).

The composition and route of delivery of METs differs based on the indication. For example, MET-2 is a lyophilized bacterial product that is given orally, in encapsulated form for UC. MET-2 for rCDI is supplied in 2 dosage forms: 1) lyophilized powder in capsules for oral ingestion and 2) lyophilized powder for rectal administration by colonoscopy (powder is resuspended in 0.9% saline). MET-1 was a live bacterial product (resuspended in 0.9% saline) also administered by colonoscopy. A recent non-inferiority trial showed that oral capsules are equally effective compared to colonoscopy-delivered FMT for rCDI. Notably, there were fewer minor adverse events in patients receiving FMT capsules compared to patients receiving FMT via colonoscopy in the above study. Additionally, several FMT studies have shown that frozen fecal material is as effective as fresh fecal material in treating rCDI. More recently, FMT has been given in lyophilized form by capsule delivery, with an 88% success rate. Accordingly, the MET-2 clinical protocol implemented changes in the route of administration with regards to these recent advances in the literature. Encapsulated lyophilized FMT material has not been studied for use in UC patients, although lyophilized bacterial product preparations are commonplace in the probiotic industry.

As described herein, MET-2 is a therapeutic composition composed of a defined microbial community of 40 bacterial strains derived from the stool of a healthy fecal donor. The bacteria are prepared as a mixture in a predetermined ratio of pure lyophilized intestinal bacteria. The bacteria are then double encapsulated in enteric capsules. MET-2 capsules contain 0.5 g of MET-2 (equivalent to 3.59×10⁷ to 3.59×10¹² CFU) and are administered to patients via oral route. The donor from where MET-2 strains were derived has been rigorously screened for infectious materials and blood-borne pathogens. Stool from this donor has also been previously used as an FMT donor to successfully treat rCDI. There is no upper toxicity limit is expected due to the safety profile of the MET-2 bacterial community.

A multi-species derivative community such as that described herein will be more generally useful than a single organism probiotic or a mixed culture of such probiotic species. The microbes in MET-2 are derived from a community and are expected to retain community structure to a degree that enables them to colonize the colonic environment. A defined microbial community, isolated from a single healthy donor, may be sufficiently robust to withstand further perturbations by antibiotics as indicated by results presented herein demonstrating augmented robustness responsiveness to perturbations. See, e.g., FIGS. 3 and 4.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

Appendix A
NB2-A29D6 Parabacteroides merdae
(SEQ ID NO: 1)
ACGAAGAGTTTGATCCTGGCTCAGGATGAACGCTAGCGACAGGCTTAACACATGCA
AGTCGAGGGGCAGCATGATTTGTAGCAATACAGATTGATGGCGACCGGCGCACGGG
TGAGTAACGCGTATGCAACTTACCTATCAGAGGGGGATAGCCCGGCGAAAGTCGGA
TTAATACCCCATAAAACAGGGGTCCCGCATGGGAATATTTGTTAAAGATTCATCGCT
GATAGATAGGCATGCGTTCCATTAGGCAGTTGGCGGGGTAACGGCCCACCAAACCG
ACGATGGATAGGGGTTCTGAGAGGAAGGTCCCCCACATTGGTACTGAGACACGGAC
CAAACTCCTACGGGAGGCAGCAGTGAGGAATATTGGTCAATGGCCGAGAGGCTGAA
CCAGCCAAGTCGCGTGAAGGAAGAAGGATCTATGGTTTGTAAACTTCTTTTATAGGG
GAATAAAGTGGAGGACGTGTCCTTTTTTGTATGTACCCTATGAATAAGCATCGGCTA
ACTCCGTGCCAGCAGCCGCGGTAATACGGAGGATGCGAGCGTTATCCGGATTTATTG
GGTTTAAAGGGTGCGTAGGTGGTGATTTAAGTCAGCGGTGAAAGTTTGTGGCTCAAC
CATAAAATTGCCGTTGAAACTGGGTTACTTGAGTGTGTTTGAGGTAGGCGGAATGCG
TGGTGTAGCGGTGAAATGCATAGATATCACGCAGAACTCCGATTGCGAAGGCAGCT
TACTAAACCATAACTGACACTGAAGCACGAAAGCGTGGGGATCAAACAGGATTAGA
TACCCTGGTAGTCCACGCAGTAAACGATGATTACTAGGAGTTTGCGATACAATGTAA
GCTCTACAGCGAAAGCGTTAAGTAATCCACCTGGGGAGTACGCCGGCAACGGTGAA
ACTCAAAGGAATTGACGGGGGCCCGCACAAGCGGAGGAACATGTGGTTTAATTCGA
TGATACGCGAGGAACCTTACCCGGGTTTGAACGTAGTCTGACCGGAGTGGAAACAC
TCCTTCTAGCAATAGCAGATTACGAGGTGCTGCATGGTTGTCGTCAGCTCGTGCCGT
GAGGTGTCGGCTTAAGTGCCATAACGAGCGCAACCCTTATCACTAGTTACTAACAGG
TGAAGCTGAGGACTCTGGTGAGACTGCCAGCGTAAGCTGTGAGGAAGGTGGGGATG
ACGTCAAATCAGCACGGCCCTTACATCCGGGGCGACACACGTGTTACAATGGCATG
GACAAAGGGCAGCTACCTGGCGACAGGATGCTAATCTCCAAACCATGTCTCAGTTC
GGATCGGAGTCTGCAACTCGACTCCGTGAAGCTGGATTCGCTAGTAATCGCGCATCA
GCCATGGCGCGGTGAATACGTTCCCGGGCCTTGTACACACCGCCCGTCAAGCCATGG
GAGCCGGGGGTACCTGAAGTCCGTAACCGCAAGGATCGGCCTAGGGTAAAACTGGT
GACTGGGGCTAAGTCGTAACAAGGTAGCCGTACCGGAAGGTGCGGCTGGAACACCT
CCTTT
NB2-B13BHI [Eubacterium] hallii
(SEQ ID NO: 2)
CTGAGTGGCGGACGGGTGAGTAACGCGTGGGTAACCTGCCCTGTACAGGGGGATAA
CAGCTGGAAACGGCTGCTAATACCGCATAAGCGCACGAGGAGACATCTCCTTGTGT
GAAAAACTCCGGTGGTACAGGATGGGCCCGCGTCTGATTAGCTGGTTGGCAGGGTA
ACGGCCTACCAAGGCAACGATCAGTAGCCGGTCTGAGAGGATGAACGGCCACATTG
GAACTGAGACACGGTCCAAACTCCTACGGGAGGCAGCAGTGGGGAATATTGCACAA
TGGGGGAAACCCTGATGCAGCAACGCCGCGTGAGTGAAGAAGTATTTCGGTATGTA
AAGCTCTATCAGCAGGGAAGATAATGACGGTACCTGACTAAGAAGCTCCGGCTAAA
TACGTGCCAGCAGCCGCGGTAATACGTATGGAGCAAGCGTTATCCGGATTTACTGGG
TGTAAAGGGTGCGTAGGTGGCAGTGCAAGTCAGATGTGAAAGGCCGGGGCTCAACC
CCGGAGCTGCATTTGAAACTGCTCGGCTAGAGTACAGGAGAGGCAGGCGGAATTCC
TAGTGTAGCGGTGAAATGCGTAGATATTAGGAGGAACACCAGTGGCGAAGGCGGCC
TGCTGGACTGTTACTGACACTGAGGCACGAAAGCGTGGGGAGCAAACAGGATTAGA
TACCCTGGTAGTCCACGCCGTAAACGATGAATACTAGGTGTCGGGGCCGTATAGGCT
TCGGTGCCGCCGCTAACGCAGTAAGTATTCCACCTGGGGAGTACGTTCGCAAGAATG
AAACTCAAAGGAATTGACGGGGACCCGCACAAGCGGTGGAGCATGTGGTTTAATTC
GAAGCAACGCGAAGAACCTTACCAGGTCTTGACATCCTTCTGACCGCACCTTAATCG
GTGCTTTCCTTCGGGACAGAAGAGACAGGTGGTGCATGGTTGTCGTCAGCTCGTGTC
GTGAGATGTTGGGTTAAGTCCCGCAACGAGCGCAACCCCTATCTTCAGTAGCCAGCA
GGTAAGGCTGGGCACTCTGGAGAGACTGCCAGGGATAACCTGGAGGAAGGTGGGG
ACGACGTCAAATCATCATGCCCCTTATGATCTGGGCGACACACGTGCTACAATGGCG
GTCACAGAGTGAGGCGAACCCGCGAGGGGGAGCAAACCACAAAAAGGCCGTCCCA
GTTCGGACTGTAGTCTGCAACCCGACTACACGAAGCTGGAATCGCTAGTAATCGCGA
ATCAGAATGTCGCGGTGAATACGTTCCCGGGTCTTGTACACACCGCCCGTCACACCA
TGGGAGTCGGAAATGCCCGAAGCCAGTGACCCAACCTTTTGGAGGGAGCTGTCGAA
GGTGGAGCCGGTAACTGGGGTGAAGTCGTAACAAGGTAGCCGTATCGGAAGGTGCG
GCTGGATCACCTCCTTT
NB2-A10MRS Parabacteroides distasonis
(SEQ ID NO: 3)
CTATCAGAGGGGGATAACCCGGCGAAAGTCGGACTAATACCGCATGAAGCAGGGGC
CCCGCATGGGGATATTTGCTAAAGATTCATCGCTGATAGATAGGCATGCGTTCCATT
AGGCAGTTGGCGGGGTAACGGCCCACCAAACCGACGATGGATAGGGGTTCTGAGAG
GAAGGTCCCCCACATTGGTACTGAGACACGGACCAAACTCCTACGGGAGGCAGCAG
TGAGGAATATTGGTCAATGGGCGTAAGCCTGAACCAGCCAAGTCGCGTGAGGGATG
AAGGTTCTATGGATCGTAAACCTCTTTTATAAGGGAATAAAGTGCGGGACGTGTCCT
GTTTTGTATGTACCTTATGAATAAGGATCGGCTAACTCCGTGCCAGCAGCCGCGGTA
ATACGGAGGATCCGAGCGTTATCCGGATTTATTGGGTTTAAAGGGTGCGTAGGCGGC
CTTTTAAGTCAGCGGTGAAAGTCTGTGGCTCAACCATAGAATTGCCGTTGAAACTGG
GGGGCTTGAGTATGTTTGAGGCAGGCGGAATGCGTGGTGTAGCGGTGAAATGCTTA
GATATCACGCAGAACCCCGATTGCGAAGGCAGCCTGCCAAGCCATGACTGACGCTG
ATGCACGAAAGCGTGGGGATCAAACAGGATTAGATACCCTGGTAGTCCACGCAGTA
AACGATGATCACTAGCTGTTTGCGATACAGTGTAAGCGGCACAGCGAAAGCGTTAA
GTGATCCACCTGGGGAGTACGCCGGCAACGGTGAAACTCAAAGGAATTGACGGGGG
CCCGCACAAGCGGAGGAACATGTGGTTTAATTCGATGATACGCGAGGAACCTTACC
CGGGTTTGAACGCATTCGGACCGAGGTGGAAACACCTTTTCTAGCAATAGCCGTTTG
CGAGGTGCTGCATGGTTGTCGTCAGCTCGTGCCGTGAGGTGTCGGCTTAAGTGCCAT
AACGAGCGCAACCCTTGCCACTAGTTACTAACAGGTGATGCTGAGGACTCTGGTGG
GACTGCCAGCGTAAGCTGCGAGGAAGGCGGGGATGACGTCAAATCAGCACGGCCCT
TACATCCGGGGCGACACACGTGTTACAATGGCGTGGACAAAGGGATGCCACCTGGC
GACAGGGAGCGAATCCCCAAACCACGTCTCAGTTCGGATCGGAGTCTGCAACCCGA
CTCCGTGAAGCTGGATTCGCTAGTAATCGCGCATCAGCCATGGCGCGGTGAATACGT
TCCCGGGCCTTGTACACACCGCCCGTCAAGCCATGGGAGCCGGGGGTACCTGAAGT
CCGTA
NB2-A12FMU Phascolarctobacterium succinatutens
(SEQ ID NO: 4)
ATTGGAGAGTTTGATCCTGGCTCAGGACGAACGCTGGCGGCATGCCTAACACATGC
AAGTCGAACGGAGAAAGTTCAACACCAAGTATTTCATCCGCTGAAGTGTAGCGGTA
AAAATTGCGAAGCAATTTTTACTACGCATTAAAAGCATGAACTAACACGGTGGTTGA
AGTATTAGGTGTTGAACTTTCTTAGTGGCGAACGGGTGAGTAACGCGTGGGCAACCT
GCCCTCTAGATGGGGACAACATCCCGAAAGGGGTGCTAATACCGAATGTGACAGCA
ATCTCGCATGAGGATGCTGTGAAAGATGGCCTCTATTTATAAGCTATCGCTAGAGGA
TGGGCCTGCGTCTGATTAGCTAGTTGGTGGGGTAACGGCCTACCAAGGCGATGATCA
GTAGCCGGTCTGAGAGGATGAACGGCCACATTGGGACTGAGACACGGCCCAGACTC
CTACGGGAGGCAGCAGTGGGGAATCTTCCGCAATGGGCGAAAGCCTGACGGAGCAA
TGCCGCGTGAGTGATGAAGGAATTCGTTCCGTAAAGCTCTTTTGTTTATGACGAATG
TGCAGATTGTAAATAATGATCTGTAATGACGGTAGTAAACGAATAAGCCACGGCTA
ACTACGTGCCAGCAGCCGCGGTAATACGTAGGTGGCGAGCGTTGTCCGGAATTATTG
GGCGTAAAGAGCATGTAGGCGGTTTTTTAAGTCTGGAGTGAAAATGCGGGGCTCAA
CCCCGTATGGCTCTGGATACTGGAAGACTTGAGTGCAGGAGAGGAAAGGGGAATTC
CCAGTGTAGCGGTGAAATGCGTAGATATTGGGAGGAACACCAGTGGCGAAGGCGCC
TTTCTGGACTGTGTCTGACGCTGAGATGCGAAAGCCAGGGTAGCGAACGGGATTAG
ATACCCCGGTAGTCCTGGCCGTAAACGATGGGTACTAGGTGTAGGAGGTATCGACC
CCTTCTGTGCCGGAGTTAACGCAATAAGTACCCCGCCTGGGGAGTACGTCCGCAAG
GATGAAACTCAAAGGAATTGACGGGGGCCCGCACAAGCGGTGGAGTATGTGGTTTA
ATTCGACGCAACGCGAAGAACCTTACCAAGGCTTGACATTGAATGACCGCTCCAGA
GATGGAGCTTTCCCTTCGGGGACATGAAAACAGGTGGTGCATGGCTGTCGTCAGCTC
GTGTCGTGAGATGTTGGGTTAAGTCCCGCAACGAGCGCAACCCCTATCCTATGTTAC
CAGCGGGTAATGCCGGGGACTCATAGGAGACTGCCAAGGACAACTTGGAGGAAGGC
GGGGATGACGTCAAGTCATCATGCCCCTTATGTCTTGGGCTACACACGTACTACAAT
GGTCGGCAACAGAGGGAAGCAAAGCCGTGAGGCAGAGCAAACCCCAGAAACCCGA
TCCCAGTTCGGATTGCAGGCTGCAACTCGCCTGCATGAAGTCGGAATCGCTAGTAAT
CGCAGGTCAGCATACTGCGGTGAATACGTTCCCGGGCCTTGTACACACCGCCCGTCA
CACCACGAAAGTTGGTAACACCCGAAGCCGGTGGGGTAACCGTAAGGAGCCAGCCG
TCTAAGGTGGGGCCGATGATTGGGGTGAAGTCGTAACAAGGTAGCCGTATCGGAAG
GTGCGGCTGGATCACCTCCTTT
NB2-B17NB Ruminococcus lactaris
(SEQ ID NO: 5)
GAGAGTTTGATCCTGGCTCAGGATGAACGCTGGCGGCGTGCTTAACACATGCAAGTC
GAGCGAAGCACTTAGGAAAGATTCTTCGGATGATTTCCTATTTGACTGAGCGGCGGA
CGGGTGAGTAACGCGTGGGTAACCTGCCTCATACAGGGGGATAACAGTTAGAAATG
ACTGCTAATACCGCATAAGACCACAGCACCGCATGGTGCAGGGGTAAAAACTCCGG
TGGTATGAGATGGACCCGCGTCTGATTAGTTAGTTGGTGGGGTAACGGCCTACCAAG
GCGACGATCAGTAGCCGACCTGAGAGGGTGACCGGCCACATTGGGACTGAGACACG
GCCCAAACTCCTACGGGAGGCAGCAGTGGGGAATATTGCACAATGGGGGAAACCCT
GATGCAGCGACGCCGCGTGAGCGAAGAAGTATTTCGGTATGTAAAGCTCTATCAGC
AGGGAAGAAAATGACGGTACCTGACTAAGAAGCCCCGGCTAACTACGTGCCAGCAG
CCGCGGTAATACGTAGGGGGCAAGCGTTATCCGGATTTACTGGGTGTAAAGGGAGC
GTAGACGGAGCAGCAAGTCTGATGTGAAAACCCGGGGCTCAACCCCGGGACTGCAT
TGGAAACTGTTGATCTGGAGTGCCGGAGAGGTAAGCGGAATTCCTAGTGTAGCGGT
GAAATGCGTAGATATTAGGAGGAACACCAGTGGCGAAGGCGGCTTACTGGACGGTA
ACTGACGTTGAGGCTCGAAAGCGTGGGGAGCAAACAGGATTAGATACCCTGGTAGT
CCACGCCGTAAACGATGACTACTAGGTGTCGGGTGGCAAAGCCATTCGGTGCCGCA
GCCAACGCAATAAGTAGTCCACCTGGGGAGTACGTTCGCAAGAATGAAACTCAAAG
GAATTGACGGGGACCCGCACAAGCGGTGGAGCATGTGGTTTAATTCGAAGCAACGC
GAAGAACCTTACCTGCTCTTGACATCCCGGTGACGGCAGAGTAATGTCTGCTTTTCT
TTGGAACACCGGTGACAGGTGGTGCATGGTTGTCGTCAGCTCGTGTCGTGAGATGTT
GGGTTAAGTCCCGCAACGAGCGCAACCCCTATCTTCAGTAGCCAGCGGTAAGGCCG
GGCACTCTGGAGAGACTGCCAGGGATAACCTGGAGGAAGGTGGGGATGACGTCAAA
TCATCATGCCCCTTATGAGCAGGGCTACACACGTGCTACAATGGCGTAAACAAAGG
GAAGCGAACCCGCGAGGGTGGGCAAATCCCAAAAATAACGTCTCAGTTCGGATTGT
AGTCTGCAACTCGACTACATGAAGCTGGAATCGCTAGTAATCGCGAATCAGAATGTC
GCGGTGAATACGTTCCCGGGTCTTGTACACACCGCCCGTCACACCATGGGAGTCAGT
AACGCCCGAAGTCAGTGACCCAACC
NB2-B16D5 Neglecta timonensis
(SEQ ID NO: 6)
TTTAGAGAGTTTGATCCTGGCTCAGGACGAACGCTGGCGGCGTGCCTAACACATGCA
AGTCGAACGGAGATAGACGCTGAAAGGGAGACAGCTTGCTGTAAGAATTTCTTGTT
TATCTTAGTGGCGGACGGGTGAGTAACGCGTGAGTAACCTGCCTTTCAGAGGGGGA
TAACGTCTGGAAACGGACGCTAATACCGCATGAGACCACAGCTTCACATGGAGCGG
CGGTCAAAGGAGCAATCCGCTGAAAGATGGACTCGCGTCCGATTAGATAGTTGGCG
GGGTAACGGCCCACCAAGTCGACGATCGGTAGCCGGACTGAGAGGTTGAACGGCCA
CATTGGGACTGAGACACGGCCCAGACTCCTACGGGAGGCAGCAGTGAGGGATATTG
GTCAATGGGGGAAACCCTGAACCAGCAACGCCGCGTGAGGGAAGACGGTTTTCGGA
TTGTAAACCTCTGTCCTCTGTGAAGATAGTGACGGTAGCAGAGGAGGAAGCTCCGG
CTAACTACGTGCCAGCAGCCGCGGTAATACGTAGGGAGCGAGCGTTGTCCGGATTT
ACTGGGTGTAAAGGGTGCGTAGGCGGCTCTGCAAGTCAGAAGTGAAATCCATGGGC
TTAACCCATGAACTGCTTTTGAAACTGTAGAGCTTGAGTGAAGTAGAGGTAGGCGG
AATTCCCGGTGTAGCGGTGAAATGCGTAGAGATCGGGAGGAACACCAGTGGCGAAG
GCGGCCTACTGGGCTTTAACTGACGCTGAGGCACGAAAGCATGGGTAGCAAACAGG
ATTAGATACCCTGGTAGTCCATGCCGTAAACGATGATTACTAGGTGTGGGGGGTCTG
ACCCCCTCCGTGCCGGAGTTAACACAATAAGTAATCCACCTGGGGAGTACGACCGC
AAGGTTGAAACTCAAAGGAATTGACGGGGGCCCGCACAAGCAGTGGAGTATGTGGA
TTAATTCGAAGCAACGCGAAGAACCTTACCAGGTCTTGACATCCAACTAACGAAGC
AGAGATGCATTAGGTGCCCTTCGGGGAAAGTTGAGACAGGTGGTGCATGGTTGTCG
TCAGCTCGTGTCGTGAGATGTTGGGTTAAGTCCCGCAACGAGCGCAACCCTTACTGT
TAGTTGCTACGCAAGAGCACTCTAGCAGGACTGCCGTTGACAAAACGGAGGAAGGT
GGGGACGACGTCAAATCATCATGCCCCTTATGACCTGGGCCTCACACGTACTACAAT
GGCCATTAACAGAGGGAAGCAAGCCCGCGAGGTGGAGCAAAACCCTAAAAATGGT
CTCAGTTCGGATCGTAGGCTGAAACCCGCCTGCGTGAAGTTGGAATTGCTAGTAATC
GCGGATCAGCATGCCGCGGTGAATACGTTCCCGGGCCTTGTACACACCGCCCGTCAC
ACCATGGGAGCCGGTAATACCCGAAGTCAGTAGTCTAACCGCAAGGGGGACGCTGC
CGAAGGTAGGATTGGCGACTGGGGTGAAGTCGTAACAAGGTAGCCGTATCGGAAGG
TGCGGCTGGATCACCTCCTTT
NB2-B10NB [Clostridium] spiroforme
(SEQ ID NO: 7)
ATGGAGAGTTTGATCCTGGCTCAGGATGAACGCTGGCGGCGTGCCTAATACATGCA
AGTCGAACGCTTCACTTCGGTGAAGAGTGGCGAACGGGTGAGTAATACATAAGTAA
CCTGGCATCTACAGGGGGATAACTGATGGAAACGTCAGCTAAGACCGCATAGGTGT
AGAGATCGCATGAACTCTATATGAAAAGTGCTACGGGACTGGTAGATGATGGACTT
ATGGCGCATTAGCTGGTTGGTAGGGTAACGGCCTACCAAGGCGACGATGCGTAGCC
GACCTGAGAGGGTGACCGGCCACACTGGGACTGAGACACGGCCCAGACTCCTACGG
GAGGCAGCAGTAGGGAATTTTCGGCAATGGGGGAAACCCTGACCGAGCAACGCCGC
GTGAAGGAAGAAGTAATTCGTTATGTAAACTTCTGTCATAGAGGAAGAACGGTGGA
TATAGGGAATGATATCCAAGTGACGGTACTCTATAAGAAAGCCACGGCTAACTACG
TGCCAGCAGCCGCGGTAATACGTAGGTGGCGAGCGTTATCCGGAATTATTGGGCGT
AAAGAGGGAGCAGGCGGCACTAAGGGTCTGTGGTGAAAGATCGAAGCTTAACTTCG
GTAAGCCATGGAAACCGTAGAGCTAGAGTGTGTGAGAGGATCGTGGAATTCCATGT
GTAGCGGTGAAATGCGTAGATATATGGAGGAACACCAGTGGCGAAGGCGACGATCT
GGCGCATAACTGACGCTCAGTCCCGAAAGCGTGGGGAGCAAATAGGATTAGATACC
CTAGTAGTCCACGCCGTAAACGATGAGTACTAAGTGTTGGGAGTCAAATCTCAGTGC
TGCAGTTAACGCAATAAGTACTCCGCCTGAGTAGTACGTTCGCAAGAATGAAACTCA
AAGGAATTGACGGGGGCCCGCACAAGCGGTGGAGCATGTGGTTTAATTCGAAGCAA
CGCGAAGAACCTTACCAGGTCTTGACATCGATCTAAAGGCTCCAGAGATGGAGAGA
TAGCTATAGAGAAGACAGGTGGTGCATGGTTGTCGTCAGCTCGTGTCGTGAGATGTT
GGGTTAAGTCCCGCAACGAGCGCAACCCCTGTTGCCAGTTGCCAGCATTAAGTTGGG
GACTCTGGCGAGACTGCCGGTGACAAGCCGGAGGAAGGCGGGGATGACGTCAAATC
ATCATGCCCCTTATGACCTGGGCTACACACGTGCTACAATGGACAGAGCAGAGGGA
AGCGAAGCCGCGAGGTGGAGCGAAACCCATAAAACTGTTCTCAGTTCGGACTGCAG
TCTGCAACTCGACTGCACGAAGATGGAATCGCTAGTAATCGCGAATCAGCATGTCGC
GGTGAATACGTTCTCGGGCCTTGTACACACCGCCCGTCACACCATGAGAGTCGGTAA
CACCCGAAGCCGGTGGCCTAACCGCAAGGAAGGAGCTGTCTAAGGTGGGACTGATG
ATTGGGGTGAAGTCGTAACAAGGTATCCCTACGGGAACGTGGGGATGGATCACCTC
CTTT
NB2-B10FAA Roseburia intestinalis
(SEQ ID NO: 8)
ACTGAGTGGCGGACGGGTGAGTAACGCGTGGGTAACCTGCCTCATACAGGGGGATA
ACAGTTGGAAACGACTGCTAATACCGCATAAGCGCACAGGGTCGCATGACCTGGTG
TGAAAAACTCCGGTGGTATGAGATGGACCCGCGTCTGATTAGCCAGTTGGTGGGGT
AACGGCCTACCAAAGCGACGATCAGTAGCCGACCTGAGAGGGTGACCGGCCACATT
GGGACTGAGACACGGCCCAAACTCCTACGGGAGGCAGCAGTGGGGAATATTGCACA
ATGGGGGAAACCCTGATGCAGCGACGCCGCGTGAGCGAAGAAGTATTTCGGTATGT
AAAGCTCTATCAGCAGGGAAGAAGAAATGACGGTACCTGACTAAGAAGCACCGGCT
AAATACGTGCCAGCAGCCGCGGTAATACGTATGGTGCAAGCGTTATCCGGATTTACT
GGGTGTAAAGGGAGCGCAGGCGGTACGGCAAGTCTGATGTGAAAGCCCGGGGCTCA
ACCCCGGTACTGCATTGGAAACTGTCGGACTAGAGTGTCGGAGGGGTAAGTGGAAT
TCCTAGTGTAGCGGTGAAATGCGTAGATATTAGGAGGAACACCAGTGGCGAAGGCG
GCTTACTGGACGATTACTGACGCTGAGGCTCGAAAGCGTGGGGAGCAAACAGGATT
AGATACCCTGGTAGTCCACGCCGTAAACGATGAATACTAGGTGTCGGGGAGCATTG
CTCTTCGGTGCCGCAGCAAACGCAATAAGTATTCCACCTGGGGAGTACGTTCGCAAG
AATGAAACTCAAAGGAATTGACGGGGACCCGCACAAGCGGTGGAGCATGTGGTTTA
ATTCGAAGCAACGCGAAGAACCTTACCAAGTCTTGACATCCCGATGACAGAACATG
TAATGTGTTTTCTCTTCGGAGCATCGGTGACAGGTGGTGCATGGTTGTCGTCAGCTC
GTGTCGTGAGATGTTGGGTTAAGTCCCGCAACGAGCGCAACCCCTATTCTTAGTAGC
CAGCGGGTAAGCCGGGCACTCTAGGGAGACTGCCAGGGATAACCTGGAGGAAGGTG
GGGATGACGTCAAATCATCATGCCCCTTATGACTTGGGCTACACACGTGCTACAATG
GCGTAAACAAAGGGAAGCGAGCCTGCGAGGGGGAGCAAATCTCAAAAATAACGTC
TCAGTTCGGACTGCAGTCTGCAACTCGACTGCACGAAGCTGGAATCGCTAGTAATCG
CGAATCAGAATGTCGCGGTGAATACGTTCCCGGGTCTTGTACACACCGCCCGTCACA
CCATGGGAGTTGGTAATGCCCGAAGTCAGTGACCCAACCGCAAGGAGGGAGCTGCC
GAAGGCAGGATCGATAACTGGGGTGAAGTCGTAACAAGGTAGCCGTATCGGAAGGT
GCGGCTGGATCACCTCCTTT
NB2-A8WC Akkermansia muciniphila
(SEQ ID NO: 9)
ATGGAGAGTTTGATTCTGGCTCAGAACGAACGCTGGCGGCGTGGATAAGACATGCA
AGTCGAACGAGAGAATTGCTAGCTTGCTAATAATTCTCTAGTGGCGCACGGGTGAGT
AACACGTGAGTAACCTGCCCCCGAGAGCGGGATAGCCCTGGGAAACTGGGATTAAT
ACCGCATAGAATCGCAAGATTAAAGCAGCAATGCGCTTGGGGATGGGCTCGCGGCC
TATTAGTTAGTTGGTGAGGTAACGGCTCACCAAGGCGATGACGGGTAGCCGGTCTG
AGAGGATGTCCGGCCACACTGGAACTGAGACACGGTCCAGACACCTACGGGTGGCA
GCAGTCGAGAATCATTCACAATGGGGGAAACCCTGATGGTGCGACGCCGCGTGGGG
GAATGAAGGTCTTCGGATTGTAAACCCCTGTCATGTGGGAGCAAATTAAAAAGATA
GTACCACAAGAGGAAGAGACGGCTAACTCTGTGCCAGCAGCCGCGGTAATACAGAG
GTCTCAAGCGTTGTTCGGAATCACTGGGCGTAAAGCGTGCGTAGGCTGTTTCGTAAG
TCGTGTGTGAAAGGCGCGGGCTCAACCCGCGGACGGCACATGATACTGCGAGACTA
GAGTAATGGAGGGGGAACCGGAATTCTCGGTGTAGCAGTGAAATGCGTAGATATCG
AGAGGAACACTCGTGGCGAAGGCGGGTTCCTGGACATTAACTGACGCTGAGGCACG
AAGGCCAGGGGAGCGAAAGGGATTAGATACCCCTGTAGTCCTGGCAGTAAACGGTG
CACGCTTGGTGTGCGGGGAATCGACCCCCTGCGTGCCGGAGCTAACGCGTTAAGCG
TGCCGCCTGGGGAGTACGGTCGCAAGATTAAAACTCAAAGAAATTGACGGGGACCC
GCACAAGCGGTGGAGTATGTGGCTTAATTCGATGCAACGCGAAGAACCTTACCTGG
GCTTGACATGTAATGAACAACATGTGAAAGCATGCGACTCTTCGGAGGCGTTACAC
AGGTGCTGCATGGCCGTCGTCAGCTCGTGTCGTGAGATGTTTGGTTAAGTCCAGCAA
CGAGCGCAACCCCTGTTGCCAGTTACCAGCACGTGAAGGTGGGGACTCTGGCGAGA
CTGCCCAGATCAACTGGGAGGAAGGTGGGGACGACGTCAGGTCAGTATGGCCCTTA
TGCCCAGGGCTGCACACGTACTACAATGCCCAGTACAGAGGGGGCCGAAGCCGCGA
GGCGGAGGAAATCCTAAAAACTGGGCCCAGTTCGGACTGTAGGCTGCAACCCGCCT
ACACGAAGCCGGAATCGCTAGTAATGGCGCATCAGCTACGGCGCCGTGAATACGTT
CCCGGGTCTTGTACACACCGCCCGTCACATCATGGAAGCCGGTCGCACCCGAAGTAT
CTGAAGCCAACCGCAAGGAGGCAGGGTCCTAAGGTGAGACTGGTAACTGGGATGAA
GTCGTAACAAGGTAGCCGTAGGGGAACCTGCGGCTGGATCACCTCCTTT
NB2-A9NA [Ruminococcus] obeum
(SEQ ID NO: 10)
GCTTAACACATGCAAGTCGAACGAGAAGGCGTAGCAATACGCTTGTAAAGTGGCGA
ACGGGTGAGNAACACNTGGGTAACCTACCCTCGAGTGGGGGATAACCCGCCGAAAG
GCGGGCTAATACCGCGTACGCTTCCGATCTTGCGAGATCGGAAGGAAAGCTGTCCC
AAGGGGATGGCGCTCAAGGATGGGCTCACGTCCNATCAGCTNGTTGGTGNGGTAAC
GGCNNACCAAGGCGACGANGGNTAGCTGGTCTGAGAGGANGANCAGCCACACTGG
GACTGNGACACGGCCCAGACTCCTACGGGAGGCAGCAGTNGGGAATCTTGCGCAAT
GNGCGAAAGCNTGACGCAGCNACGCCGCGTGNGGGANGANGGCCNTCGGGTTGTA
AACCNCTTTCAGNAGGGACGAATCTGACGGTACCTGCAGAAGAAGCCCCGGCNAAC
TACGTGCCAGCAGCCGCGGTAATACGTAGGGGGCNAGCGTTGTCCGGATTTATTGG
GCGTAAAGAGCTCGTAGGCGGCTTGGCAAGTCGGGTGTGAAACCTCCAGGCTTAAC
CTGGAGACGCCACTCGATNCTGCCATGGCTAGAGTCCGGTAGGGGACCACGGAATT
CCTGGTGTAGCGGTGAAATGCGCAGATATCAGGAGGAACNCCGGTGGCGAAGGCGG
NGNTCTGGGNCGGNACTGACGCTGAGGNGCGAAAGCGTGGGNAGCAAACAGGATT
AGATACCCTGGTAGTCCACGCCGTAAACGNTGGGCACTAGGTGTGGGACCTTATCA
ACGGGTTCCGTGCCGTAGCTAACGCATTAAGTGCCCCGCCTGGGGAGTACGGCCGC
AAGGCTAAAACTCAAAGGAATTGACGGGGGCCCGCACAAGCGGCGGAGCATGTTGC
TTAATTCGATGCAACGCGAAGAACCTTACCTGGGTTGAACTACGCGGGAAAAGCCA
CAGAGATGTGGTGTCCGAAAGGGCCCGCGATAGGTGGTGCATGGCTGTCGTCAGCT
CGTGTCGTGAGATGTTGGGTTAAGTCCCGCAACGAGCGCAACCCTTNTCCNATGTTG
CCAGCGGATCATGCCGGGGACTCNTGGGAGACTGCCGGGGTCAACTCGGAGGAAGG
TGGGGATGACGTCAAGTCANCATGCCCCTTATGTCCAGGGCTNNAAACATGCTACA
ATGGCCGGTACAAAGGGTNGCGAGNCNGCGANGNNGAGCNAATCCCATAAAGNNN
GTCTNAGTNCGGATCGNAGTCTGCAACTCGACTNCGTGAAGNCGGAGTNGCTAGTA
ATCNCGNATCAGCANNGNCGNGGTGAATACGTTCCCGGGCCTTGTACACACCGCCC
GTCACACCACGAAAGTTGGTAACACCCGAAGCCGGTGG
NB2-B20GAM [Clostridium] lactatifermentans
(SEQ ID NO: 11)
GAGTAATTCGGTATAGGATGGGCCCGCATCTGATTAGCTAGTTGGTGAGATAACAGC
CCACCAAGGCGACGATCAGTAGCCGACCTGAGAGGGTGATCGGCCACATTGGGACT
GAGACACGGCCCAAACTCCTACGGGAGGCAGCAGTGGGGAATATTGCACAATGGGG
GAAACCCTGATGCAGCAACGCCGCGTGAAGGAAGAAGGTTTTCGGATCGTAAACTT
CTATCAACAGGGACGAAGAAAGTGACGGTACCTGAATAAGAAGCCCCGGCTAACTA
CGTGCCAGCAGCCGCGGTAATACGTAGGGGGCAAGCGTTATCCGGAATTACTGGGT
GTAAAGGGAGCGTAGGCGGCACGCCAAGCCAGATGTGAAAGCCCGAGGCTTAACCT
CGCGGATTGCATTTGGAACTGGCGAGCTAGAGTACAGGAGAGGAAAGCGGAATTCC
TAGTGTAGCGGTGAAATGCGTAGATATTAGGAAGAACACCAGTGGCGAAGGCGGCT
TTCTGGACTGAAACTGACGCTGAGGCTCGAAAGCGTGGGGAGCAAACAGGATTAGA
TACCCTGGTAGTCCACGCCGTAAACGATGAGTGCTAGGTGTCGGGGAGGAATCCTC
GGTGCCGCAGCTAACGCAATAAGCACTCCACCTGGGGAGTACGACCGCAAGGTTGA
AACTCAAAGGAATTGACGGGGGCCCGCACAAGCGGTGGAGCATGTGGTTTAATTCG
AAGCAACGCGAAGAACCTTACCAAGGCTTGACATCCCGATGACCGCTCTAGAGATA
GAGNTTCTCTTCGGAGCATCGGTGACAGGTGGTGCATGGTTGTCGTCAGCTCGTGTC
GTGAGATGTTGGGTTAAGTCCCGCAACGAGCGCAACCCTTATCCTTAGTAGCCATCA
TTGAGTTGGGCACTCTAGGGAGACTGCCGTGGATAACACGGAGGAAGGTGGGGATG
ACGTCAAATCATCATGCCCCTTATGTCTTGGGCTACACACGTGCTACAATGGCTGGT
AACAGAGTGAAGCGAGACGGCGACGTTAAGCAAATCACAAAAACCCAGTCCCAGTT
CGGATTGTAGTCTGCAACTCGACTACATGAAGCTGGAATCGCTAGTAATCGCGAATC
AGAATGTCGCGGTGAATACGTTCCCGGGCCTTGTACACACCGCCCGTCACACCATGG
GAGTTGGAAGCACCCGAAGTCGGTGACCTAACCGTAAGGAAGGAGCCGCCGAAGGT
GAAGCCAGTGACTGGGGTGAAGTCGTAACAAGGTAGCCGTATCGGAAGGTGCGGCT
GGATCACCTCCTTT
NB2-A15BHI Anaerovorax odorimutans
(SEQ ID NO: 12)
ATATGAGAGTTTGATCCTGGCTCAGGATGAACGCTGGCGGCGTGCCTAACACATGCA
AGTCGAGCGAGAAGCTGATGATTGACACTTCGGTTGAGAGAATCAGTGGAAAGCGG
CGGACGGGTGAGTAACGCGTAGGCAACCTGCCCTTTGCAGAGGGATAGCCTCGGGA
AACCGGGATTAAAACCTCATGATGCTGTATGTCCGCATGGGCAGACGGTCAAAGAT
TTATCGGCAGAGGATGGGCCTGCGTCTGATTAGTTAGTTGGTGGGGTAACGGCCTAC
CAAGGCAACGATCAGTAGCCGACCTGAGAGGGTGATCGGCCACATTGGAACTGAGA
CACGGTCCAAACTCCTACGGGAGGCAGCAGTGGGGAATATTGCACAATGGGGGAAA
CCCTGATGCAGCAACGCCGCGTGAGCGAAGAAGGCCTTTGGGTCGTAAAGCTCTGT
CCTTGGGGAAGAAAAAATGACGGTACCCAAGGAGGAAGCCCCGGCTAACTACGTGC
CAGCAGCCGCGGTAATACGTAGGGGGCAAGCGTTATCCGGAATTATTGGGCGTAAA
GAGTATGTAGGTGGTTTCTTAAGCGCAGGGTATAAGGCAATGGCTTAACCATTGTTC
GCCCCGTGAACTGAGAGACTTGAGTGCTGGAGAGGAAAGCGGAATTCCTAGTGTAG
CGGTGAAATGCGTAGATATTAGGAGGAACACCAGTGGCGAAGGCGGCTTTCTGGAC
AGTAACTGACACTGAGATACGAAAGCGTGGGGAGCAAACAGGATTAGATACCCTGG
TAGTCCACGCCGTAAACGATGAGCACTAGGTGTCGGGCTCGCAAGAGTTCGGTGCC
GGAGTTAACGCATTAAGTGCTCCGCCTGGGGAGTACGCACGCAAGTGTAAAACTCA
AAGGAATTGACGGGGACCCGCACAAGCAGCGGAGCATGTGGTTTAATTCGAAGCAA
CGCGAAGAACCTTACCAGGGCTTGACATCCCTCCGACCGGTCCTTAATCGGACCTTT
CTACGGACGGGGGAGACAGGTGGTGCATGGTTGTCGTCAGCTCGTGTCGTGAGATG
TTGGGTTAAGTCCCGCAACGAGCGCAACCCTTGTCATTAGTTGCTAACAGTAAGATG
AGAACTCTAATGAGACTGCCGTGGATAACACGGAGGAAGGTGGGGATGACGTCAAA
TCATCATGCCCCTTATGTCCTGGGCTACACACGTGCTACAATGGTCGGTACAAAGAG
AAGCAAGACCGCGAGGTGGAGCAAATCTCAAAAACCGATCCCAGTTCGGATTGCAG
GCTGCAACTCGCCTGCATGAAGTCGGAGTTGCTAGTAATCGCAGATCAGAATGCTGC
GGTGAATGCGTTCCCGGGTCTTGTACACACCGCCCGTCACACCATGGAAGTTGGGGG
CGCCCGAAGTCGGCTAGTAAATAGGCTGCCTAAGGCGAAATCAATGACTGGGGTGA
AGTCGTAACAAGGTAGCCGTATCGGAAGGTGCGGCTGGATCACCTCCTTT
NB2-A14FMU [Ruminococcus] torques
(SEQ ID NO: 13)
AACGAGAGTTTGATCCTGGCTCAGGATGAACGCTGGCGGCGTGCCTAACACATGCA
AGTCGAGCGAAGCACTTTGCTTAGATTCTTCGGATGAAGAGGATTGTGACTGAGCGG
CGGACGGGTGAGTAACGCGTGGGTAACCTGCCTCATACAGGGGGATAACAGTTAGA
AATGACTGCTAATACCGCATAAGACCACAGCACCGCATGGTGCGGGGGTAAAAACT
CCGGTGGTATGAGATGGACCCGCGTCTGATTAGCTAGTTGGTAAGGTAACGGCTTAC
CAAGGCGACGATCAGTAGCCGACCTGAGAGGGTGACCGGCCACATTGGGACTGAGA
CACGGCCCAAACTCCTACGGGAGGCAGCAGTGGGGAATATTGCACAATGGGGGAAA
CCCTGATGCAGCGACGCCGCGTGAGCGAAGAAGTATTTCGGTATGTAAAGCTCTATC
AGCAGGGAAGAAAATGACGGTACCTGACTAAGAAGCACCGGCTAAATACGTGCCAG
CAGCCGCGGTAATACGTATGGTGCAAGCGTTATCCGGATTTACTGGGTGTAAAGGG
AGCGTAGACGGATGGGCAAGTCTGATGTGAAAACCCGGGGCTCAACCCCGGGACTG
CATTGGAAACTGTTCATCTAGAGTGCTGGAGAGGTAAGTGGAATTCCTAGTGTAGCG
GTGAAATGCGTAGATATTAGGAGGAACACCAGTGGCGAAGGCGGCTTACTGGACAG
TAACTGACGTTGAGGCTCGAAAGCGTGGGGAGCAAACAGGATTAGATACCCTGGTA
GTCCACGCCGTAAACGATGACTACTAGGTGTCGGGTGGCAAAGCCATTCGGTGCCG
CAGCAAACGCAATAAGTAGTCCACCTGGGGAGTACGTTCGCAAGAATGAAACTCAA
AGGAATTGACGGGGACCCGCACAAGCGGTGGAGCATGTGGTTTAATTCGAAGCAAC
GCGAAGAACCTTACCTGCTCTTGACATCCCGCTGACCGGACGGTAATGCGTCCTTCC
CTTCGGGGCAGCGGAGACAGGTGGTGCATGGTTGTCGTCAGCTCGTGTCGTGAGATG
TTGGGTTAAGTCCCGCAACGAGCGCAACCCCTATCTTTAGTAGCCAGCGGCCAGGCC
GGGCACTCTAGAGAGACTGCCGGGGATAACCCGGAGGAAGGTGGGGATGACGTCA
AATCATCATGCCCCTTATGAGCAGGGCTACACACGTGCTACAATGGCGTAAACAAA
GGGAAGCGAGACCGCGAGGTGGAGCAAATCCCAAAAATAACGTCTCAGTTCGGATT
GTAGTCTGCAACTCGACTACATGAAGCTGGAATCGCTAGTAATCGCGAATCAGAAT
GTCGCGGTGAATACGTTCCCGGGTCTTGTACACACCGCCCGTCACACCATGGGAGTC
AGTAACGCCCGAAGTCAGTGACCCAACCGTAAGGAGGGAGCTGCCGAAGGCGG
NB2-A17FMU [Eubacterium] rectale
(SEQ ID NO: 14)
ATTTTGTGACTGAGTGGCGGACGGGTGAGTAACGCGTGGGTAACCTGCCTTGTACAG
GGGGATAACAGTTGGAAACGGCTGCTAATACCGCATAAGCGCACAGCATCGCATGA
TGCAGTGTGAAAAACTCCGGTGGTATAAGATGGACCCGCGTTGGATTAGCTAGTTGG
TGAGGTAACGGCCCACCAAGGCGACGATCCATAGCCGACCTGAGAGGGTGACCGGC
CACATTGGGACTGAGACACGGCCCAAACTCCTACGGGAGGCAGCAGTGGGGAATAT
TGCACAATGGGCGAAAGCCTGATGCAGCGACGCCGCGTGAGCGAAGAAGTATTTCG
GTATGTAAAGCTCTATCAGCAGGGAAGATAATGACGGTACCTGACTAAGAAGCACC
GGCTAAATACGTGCCAGCAGCCGCGGTAATACGTATGGTGCAAGCGTTATCCGGATT
TACTGGGTGTAAAGGGAGCGCAGGCGGTGCGGCAAGTCTGATGTGAAAGCCCGGGG
CTCAACCCCGGTACTGCATTGGAAACTGTCGTACTAGAGTGTCGGAGGGGTAAGCG
GAATTCCTAGTGTAGCGGTGAAATGCGTAGATATTAGGAGGAACACCAGTGGCGAA
GGCGGCTTACTGGACGATAACTGACGCTGAGGCTCGAAAGCGTGGGGAGCAAACAG
GATTAGATACCCTGGTAGTCCACGCCGTAAACGATGAATACTAGGTGTTGGGAAGC
ATTGCTTCTCGGTGCCGTCGCAAACGCAGTAAGTATTCCACCTGGGGAGTACGTTCG
CAAGAATGAAACTCAAAGGAATTGACGGGGACCCGCACAAGCGGTGGAGCATGTG
GTTTAATTCGAAGCAACGCGAAGAACCTTACCAAGTCTTGACATCCTTCTGACCGGT
ACTTAACCGTACCTTCTCTTCGGAGCAGGAGTGACAGGTGGTGCATGGTTGTCGTCA
GCTCGTGTCGTGAGATGTTGGGTTAAGTCCCGCAACGAGCGCAACCCTTATCTTTAG
TAGCCAGCGGTTCGGCCGGGCACTCTAGAGAGACTGCCAGGGATAACCTGGAGGAA
GGCGGGGATGACGTCAAATCATCATGCCCCTTATGACTTGGGCTACACACGTGCTAC
AATGGCGTAAACAAAGGGAAGCAAAGCTGTGAAGCCGAGCAAATCTCAAAAATAA
CGTCTCAGTTCGGACTGTAGTCTGCAACCCGACTACACGAAGCTGGAATCGCTAGTA
ATCGCAGATCAGAATGCTGCGGTGAATACGTTCCCGGGTCTTGTACACACCGCCCGT
CACACCATGGGAGTTGGGAATGCCCGAAGCCAGTGACCTAACCGAAAGGAAGGAG
CTGTCGAAGGCAGGCTCGATAACTGGGGTGAAGTCGTAACAAGGTAGCCGTATCGG
AAGGTGCGGCTGGATCACCTCCTTT
NB2-B14D5 Bacteroides eggerthii
(SEQ ID NO: 15)
ATGAAGAGTTTGATCCTGGCTCAGGATGAACGCTAGCTACAGGCTTAACACATGCA
AGTCGAGGGGCAGCATGATTGAAGCTTGCTTCAATCGATGGCGACCGGCGCACGGG
TGAGTAACACGTATCCAACCTGCCGATAACTCGGGGATAGCCTTTCGAAAGAAAGA
TTAATACCCGATAGTATAGTATTTCCGCATGGTTTCACTATTAAAGAATTTCGGTTAT
CGATGGGGATGCGTTCCATTAGATAGTTGGCGGGGTAACGGCCCACCAAGTCAACG
ATGGATAGGGGTTCTGAGAGGAAGGTCCCCCACATTGGAACTGAGACACGGTCCAA
ACTCCTACGGGAGGCAGCAGTGAGGAATATTGGTCAATGGACGAGAGTCTGAACCA
GCCAAGTAGCGTGAAGGATGACTGCCCTATGGGTTGTAAACTTCTTTTATACGGGAA
TAAAGTGGAGTATGCATACTCCTTTGTATGTACCGTATGAATAAGGATCGGCTAACT
CCGTGCCAGCAGCCGCGGTAATACGGAGGATCCGAGCGTTATCCGGATTTATTGGGT
TTAAAGGGAGCGTAGGCGGGTGCTTAAGTCAGTTGTGAAAGTTTGCGGCTCAACCGT
AAAATTGCAGTTGATACTGGGTACCTTGAGTGCAGCATAGGTAGGCGGAATTCGTG
GTGTAGCGGTGAAATGCTTAGATATCACGAAGAACTCCGATTGCGAAGGCAGCTTA
CTGGACTGTAACTGACGCTGATGCTCGAAAGTGTGGGTATCAAACAGGATTAGATA
CCCTGGTAGTCCACACAGTAAACGATGAATACTCGCTGTTGGCGATACACAGTCAGC
GGCCAAGCGAAAGCATTAAGTATTCCACCTGGGGAGTACGCCGGCAACGGTGAAAC
TCAAAGGAATTGACGGGGGCCCGCACAAGCGGAGGAACATGTGGTTTAATTCGATG
ATACGCGAGGAACCTTACCCGGGCTTAAATTGCAGCGGAATGTAGTGGAAACATTA
CAGCCTTCGGGCCGCTGTGAAGGTGCTGCATGGTTGTCGTCAGCTCGTGCCGTGAGG
TGTCGGCTTAAGTGCCATAACGAGCGCAACCCTTATCTATAGTTACTATCAGGTCAT
GCTGAGGACTCTATGGAGACTGCCGTCGTAAGATGTGAGGAAGGTGGGGATGACGT
CAAATCAGCACGGCCCTTACGTCCGGGGCTACACACGTGTTACAATGGGGGGTACA
GAAGGCAGCTACCTGGCGACAGGATGCTAATCCCTAAAACCTCTCTCAGTTCGGATT
GGAGTCTGCAACCCGACTCCATGAAGCTGGATTCGCTAGTAATCGCGCATCAGCCAC
GGCGCGGTGAATACGTTCCCGGGCCTTGTACACACCGCCCGTCAAGCCATGAAAGC
CGGGGGTACCTGAAGTACGTAACCGCAAGGAGCGTCCTAGGGTAAAACTGGTGATT
GGGGCTAAGTCGTAACAAGGTAGCCGTACCGGAAGGTGCGGCTGGAACACCTCCTT
T
NB2-B26FMU Roseburia inulinivorans
(SEQ ID NO: 16)
ACTGAGTGGCGGACGGGTGAGTAACGCGTGGGTAACCTGCCTCACACAGGGGGATA
ACAGTTGGAAACGGCTGCTAATACCGCATAAGCGCACAGTACCGCATGGTACAGTG
TGAAAAACTCCGGTGGTGTGAGATGGACCCGCGTCTGATTAGCTAGTTGGCAGGGC
AACGGCCTACCAAGGCGACGATCAGTAGCCGACCTGAGAGGGTGACCGGCCACATT
GGGACTGAGACACGGCCCAAACTCCTACGGGAGGCAGCAGTGGGGAATATTGCACA
ATGGGGGAAACCCTGATGCAGCGACGCCGCGTGAGCGAAGAAGTATTTCGGTATGT
AAAGCTCTATCAGCAGGGAAGAAGAAATGACGGTACCTGACTAAGAAGCACCGGCT
AAATACGTGCCAGCAGCCGCGGTAATACGTATGGTGCAAGCGTTATCCGGATTTACT
GGGTGTAAAGGGAGCGCAGGCGGAAGGCTAAGTCTGATGTGAAAGCCCGGGGCTCA
ACCCCGGTACTGCATTGGAAACTGGTCATCTAGAGTGTCGGAGGGGTAAGTGGAAT
TCCTAGTGTAGCGGTGAAATGCGTAGATATTAGGAGGAACACCAGTGGCGAAGGCG
GCTTACTGGACGATAACTGACGCTGAGGCTCGAAAGCGTGGGGAGCAAACAGGATT
AGATACCCTGGTAGTCCACGCCGTAAACGATGAATACTAGGTGTCGGAAAGCACAG
CTTTTCGGTGCCGCCGCAAACGCATTAAGTATTCCACCTGGGGAGTACGTTCGCAAG
AATGAAACTCAAAGGAATTGACGGGGACCCGCACAAGCGGTGGAGCATGTGGTTTA
ATTCGAAGCAACGCGAAGAACCTTACCAAGTCTTGACATCCCGGTGACCGGACAGT
AATGTGTCCTTTTCTTCGGAACACCGGTGACAGGTGGTGCATGGTTGTCGTCAGCTC
GTGTCGTGAGATGTTGGGTTAAGTCCCGCAACGAGCGCAACCCTTATCCCCAGTAGC
CAGCATTTTGGATGGGCACTCTGAGGAGACTGCCAGGGATAACCTGGAGGAAGGTG
GGGATGACGTCAAATCATCATGCCCCTTATGACTTGGGCTACACACGTGCTACAATG
GCGTAAACAAAGGGAAGCGAGACCGTGAGGTGGAGCAAATCCCAAAAATAACGTC
TCAGTTCGGACTGTAGTCTGCAACCCGACTACACGAAGCTGGAATCGCTAGTAATCG
CAGATCAGAATGCTGCGGTGAATACGTTCCCGGGTCTTGTACACACCGCCCGTCACA
CCATGGGAGTTGGAAATGCCCGAAGTCAGTGACCCAACCGCAAGGAGGGAGCTGCC
GAAGGCAGGTTCGATAACTGGGGTGAAGTCGTAACAAGGTAGCCGTATCGGAAGGT
GCGGCTGGATCACCTCCTTT
NB2-B20DS [Clostridium] hylemonae
(SEQ ID NO: 17)
ACGAGAGTTTGATCCTGGCTCAGGACGAACGCTGGCGGCGTGCCTAACACATGCAA
GTCGAACGAGAATCTTTGGGATGATTCTTTCGGGATGAATTCCAAAGAGGAAAGTG
GCGGACGGGCGAGTAACGCGTGAGTAACCTGCCCATAAGAGGGGGATAATCCATGG
AAACGTGGACTAATACCGCATATTGTAGTTAAGTTGCATGACTTGATTATGAAAGAT
TTATCGCTTATGGATGGACTCGCGTCAGATTAGATAGTTGGTGAGGTAACGGCTCAC
CAAGTCAACGATCTGTAGCCGAACTGAGAGGTTGATCGGCCGCATTGGGACTGAGA
CACGGCCCAGACTCCTACGGGAGGCAGCAGTGGGGAATATTGCGCAATGGGGGCAA
CCCTGACGCAGCAACGCCGCGTGCAGGAAGAAGGTCTTCGGATTGTAAACTGTTGTC
GCAAGGGAAGAAGACAGTGACGGTACCTTGTGAGAAAGTCACGGCTAACTACGTGC
CAGCAGCCGCGGTAATACGTAGGTGACAAGCGTTGTCCGGATTTACTGGGTGTAAA
GGGCGCGTAGGCGGACTGTCAAGTCAGTCGTGAAATACCGGGGCTTAACCCCGGGG
CTGCGATTGAAACTGACAGCCTTGAGTATCGGAGAGGAAAGCGGAATTCCTAGTGT
AGCGGTGAAATGCGTAGATATTAGGAGGAACACCAGTGGCGAAGGCGGCTTTCTGG
ACGACAACTGACGCTGAGGCGCGAAAGTGTGGGGAGCAAACAGGATTAGATACCCT
GGTAGTCCACACCGTAAACGATGGATACTAGGTGTAGGAGGTATCGACCCCTTCTGT
GCCGCAGTTAACACAATAAGTATCCCACCTGGGGAGTACGACCGCAAGGTTGAAAC
TCAAAGGAATTGACGGGGGCCCGCACAAGCAGTGGAGTATGTGGTTTAATTCGAAG
CAACGCGAAGAACCTTACCTGGGCTTGACATCCCTGGAATCGAGTAGAGATACTTG
AGTGCCTTCGGGAATCAGGTGACAGGTGGTGCATGGTTGTCGTCAGCTCGTGTCGTG
AGATGTTGGGTTAAGTCCCGCAACGAGCGCAACCCCTATTGTCAGTTGCCATCATTA
AGTTGGGCACTCTGGCGAGACTGCCGGTGACAAATCGGAGGAAGGTGGGGACGACG
TCAAATCATCATGCCCCTTATGCCCAGGGCTACACACGTACTACAATGGCCGATAAC
AAAGTGCAGCGAAACCGTGAGGTGGAGCGAATCACAAAACTCGGTCTCAGTTCAGA
TTGCAGGCTGCAACTCGCCTGCATGAAGTTGGAATTGCTAGTAATCGCGGATCAGAA
TGCCGCGGTGAATACGTTCCCGGGCCTTGTACACACCGCCCGTCACACCATGAGAGT
CGATAACACCCGAAGCCTGTGAGCTAACCTTTTAGGAGGCAGCAGTCGAAGGTGGG
GTTGATGATTGGGGTGAAGTCGTAACAAGGTAGCCGTATCGGAAGGTGCGGCTGGA
TCACCTCCTTT
NB2-B3WC Barnesiella intestinihominis
(SEQ ID NO: 18)
CGGCGACCGGCGCACGGGTGAGTAACACGTATGCAATCCACCTGTAACAGGGGGAT
AACCCGGAGAAATCCGGACTAATACCCCATAATATGGGCGCTCCGCATGGAGAGCC
CATTAAAGAGAGCAATCTTGGTTACAGACGAGCATGCGCTCCATTAGCCAGTTGGCG
GGGTAACGGCCCACCAAGGCGACGATGGATAGGGGTTCTGAGAGGAAGGTCCCCCA
CATTGGAACTGAGACACGGTCCAAACTCCTACGGGAGGCAGCAGTGAGGAATATTG
GTCAATGGTCGGCAGACTGAACCAGCCAAGTCGCGTGAGGGAAGACGGCCCTACGG
GTTGTAAACCTCTTTTGTCGGAGAGTAAAGTACGCTACGTGTAGCGTATTGCAAGTA
TCCGAAGAAAAAGCATCGGCTAACTCCGTGCCAGCAGCCGCGGTAATACGGAGGAT
GCAAGCGTTATCCGGATTTATTGGGTTTAAAGGGTGCGTAGGCGGCACGCCAAGTCA
GCGGTGAAATTTCCGGGCTCAACCCGGAGTGTGCCGTTGAAACTGGCGAGCTAGAG
TGCACAAGAGGCAGGCGGAATGCGTGGTGTAGCGGTGAAATGCATAGATATCACGC
AGAACCCCGATTGCGAAGGCAGCCTGCTAGGGTGAAACAGACGCTGAGGCACGAA
AGCGTGGGTATCGAACAGGATTAGATACCCTGGTAGTCCACGCAGTAAACGATGAA
TACTAACTGTTTGCGATACAATGTAAGCGGTACAGCGAAAGCGTTAAGTATTCCACC
TGGGGAGTACGCCGGCAACGGTGAAACTCAAAGGAATTGACGGGGGCCCGCACAA
GCGGAGGAACATGTGGTTTAATTCGATGATACGCGAGGAACCTTACCCGGGCTCAA
ACGCAGGGGGAATATATATGAAAGTATATAGCTAGCAATAGTCACCTGCGAGGTGC
TGCATGGTTGTCGTCAGCTCGTGCCGTGAGGTGTCGGCTTAAGTGCCATAACGAGCG
CAACCCCTATGGACAGTTACTAACGGGTGAAGCCGAGGACTCTGTCGAGACTGCCG
GCGCAAGCCGCGAGGAAGGTGGGGATGACGTCAAATCAGCACGGCCCTTACGTCCG
GGGCGACACACGTGTTACAATGGCAGGTACAGAAGGCAGCCAGTCAGCAATGACGC
GCGAATCCCGAAAACCTGTCTCAGTTCGGATTGGAGTCTGCAACCCGACTCCATGAA
GCTGGATTCGCTAGTAATCGCGCATCAGCCATGGCGCGGTGAATACGTTCCCGGGCC
TTGTACACACCGCCCGTCAAGCCATGGAAGCCGGGAGTACCTGAAGCATGCAACCG
CAAGGAGCGTACGAAGGTAATACCGGTAACTGGGGCTAAGTCGTAACAAGGTAGCC
GTACCGGAAGGTGCGGCTGGAACACCTCCTTT
NB2-A14DS [Clostridium] aerotolerans
(SEQ ID NO: 19)
TTCCTTAGAAAGGAGGTGATCCAGCCGCACCTTCCGATACGGCTACCTTGTTACGAC
TTCACCCCAGTTATCAGTCCCGCCTTCGGCAGCTCCCTCCTTRCGGTTGGGTCACTGA
CTTCGGGCGTTACCAACTCCCATGGTGTGACGGGCGGTGTGTACAAGACCCGGGAA
CGTATTCACCGCGACATTCTGATTCGCGATTACTAGCGATTCCAGCTTCATGTAGTCG
AGTTGCAGACTACAATCCGAACTGAGACGTTATTTTTGAGATTTGCTTAAGCTCACA
CTCTCGCTTCCCTTTGTTTACGCCATTGTAGCACGTGTGTAGCCCAAGTCATAAGGGG
CATGATGATTTGACGTCATCCCCACCTTCCTCCAGGTTATCCCTGGCAGTCTCTCCAG
AGTGCCCGACCGAATCGCTGGCTACTGAAGATAAGGGTTGCGCTCGTTGCGGGACTT
AACCCAACATCTCACGACACGAGCTGACGACAACCATGCACCACCTGTCACCGATG
CTCCGAAGAGAAGGYYCCATTACRRACCGGTCATCGGGATGTCAAGACTTGGTAAG
GTTCTTCGCGTTGCTTCGAATTAAACCACATGCTCCACCGCTTGTGCGGGTCCCCGTC
AATTCCTTTGAGTTTCATTCTTGCGAACGTACTCCCCAGGTGGAATACTTATTGCGTT
TGCGGCGGCACCGAAGAGCTGTGCTCCCCGACACCTAGTATTCATCGTTTACGGCGT
GGACTACCAGGGTATCTAATCCTGTTTGCTCCCCACGCTTTCGAGCCTCAACGTCAG
TTACTGTCCAGTAAGCCGCCTTCGCCACTGGTGTTCCTCCTAATATCTACGCATTTCA
CCGCTACACTAGGAATTCCGCTTACCTCTCCAGCACTCTAGCCAAACAGTTTCAAAA
GCAGTCCCGGGGTTGAGCCCCAGCCTTTCACTTCTGACTTGCTTARCCGTCTACGCTC
CCTTTACACCCAGTAAATCCGGATAACGCTTGCCCCCTACGTATTACCGCGGCTGCT
GGCACGTAGTTAGCCGGGGCTTCTTAGTCAGGTACCGTCATTTTCTTCCCTGCTGATA
GAGCTTTACATACCGAAATACTTCTTCACTCACGCGGCGTCGCTGCATCAGGGTTTC
CCCCATTGTGCAATATTCCCCACTGCTGCCTCCCGTAGGAGTTTGGGCCGTGTCTCA
GTCCCAATGTGGCCGTTCACCCTCTCAGGCCGGCTATGGATCGTCGCCTTGGTAGGC
CGTTACCCTGCCAACTAGCTAATCCAACGCGGGTCCATCTCACACCGATAAATCTTT
TCCGTCCGGGCCATGCGGCCCTAGCGGGTTATGCGGTATTAGCGGTCGTTTCCAACT
GTTATCCCCCTGTGTGAGGCAGGTTACCCACGCGTTACTCACCCGTCCGCCACTAAG
TCGCAAGAGAAATCATCCGAAGAATCAATCTCAAGCGCTTCGTTCGACTTGCATGTG
TTAAGCACGCCGCCAGCGTTCATCCTGAGCCAGGATCAAACTCTCGATTAA
NB2-A15DCM Bacteroides stercorirosoris
(SEQ ID NO: 20)
ATGAAGAGTTTGATCCTGGCTCAGGATGAACGCTAGCTACAGGCTTAACACATGCA
AGTCGAGGGGCAGCATGACCTAGCAATAGGTTGATGGCGACCGGCGCACGGGTGAG
TAACACGTATCCAACCTACCGGTTATTCCGGGATAGCCTTTCGAAAGAAAGATTAAT
ACCGGATAGTATAACGAGAAGGCATCTTCTTGTTATTAAAGAATTTCGATAACCGAT
GGGGATGCGTTCCATTAGTTTGTTGGCGGGGTAACGGCCCACCAAGACATCGATGG
ATAGGGGTTCTGAGAGGAAGGTCCCCCACATTGGAACTGAGACACGGTCCAAACTC
CTACGGGAGGCAGCAGTGAGGAATATTGGTCAATGGACGAGAGTCTGAACCAGCCA
AGTAGCGTGAAGGATGACTGCCCTATGGGTTGTAAACTTCTTTTATATGGGAATAAA
GTGAGCCACGTGTGGCTTTTTGTATGTACCATACGAATAAGGATCGGCTAACTCCGT
GCCAGCAGCCGCGGTAATACGGAGGATCCGAGCGTTATCCGGATTTATTGGGTTTAA
AGGGAGCGTAGGCGGACTATTAAGTCAGCTGTGAAAGTTTGCGGCTCAACCGTAAA
ATTGCAGTTGATACTGGTCGTCTTGAGTGCAGTAGAGGTAGGCGGAATTCGTGGTGT
AGCGGTGAAATGCTTAGATATCACGAAGAACTCCGATTGCGAAGGCAGCTTACTGG
ACTGTAACTGACGCTGATGCTCGAAAGTGTGGGTATCAAACAGGATTAGATACCCTG
GTAGTCCACACAGTAAACGATGAATACTCGCTGTTTGCGATATACAGCAAGCGGCC
AAGCGAAAGCATTAAGTATTCCACCTGGGGAGTACGCCGGCAACGGTGAAACTCAA
AGGAATTGACGGGGGCCCGCACAAGCGGAGGAACATGTGGTTTAATTCGATGATAC
GCGAGGAACCTTACCCGGGCTTAAATTGCAAATGAATATAGTGGAAACATTATAGC
CGCAAGGCATTTGTGAAGGTGCTGCATGGTTGTCGTCAGCTCGTGCCGTGAGGTGTC
GGCTTAAGTGCCATAACGAGCGCAACCCTTATCTTTAGTTACTAACAGGTCATGCTG
AGGACTCTAGAGAGACTGCCGTCGTAAGATGTGAGGAAGGTGGGGATGACGTCAAA
TCAGCACGGCCCTTACGTCCGGGGCTACACACGTGTTACAATGGGGGGTACAGAAG
GCAGCTACACAGCGATGTGATGCTAATCCCAAAAGCCTCTCTCAGTTCGGATTGGAG
TCTGCAACCCGACTCCATGAAGCTGGATTCGCTAGTAATCGCGCATCAGCCACGGCG
CGGTGAATACGTTCNCGGGCCTTGTACACACCGCCCGTCAAGCCATGAAAGCCGGG
GGTACCTGAAGTCCGTAACCGCAAGGAG
NB2-A2OGAM Flavonifractor plautii
(SEQ ID NO: 21)
TATTGAGAGTTTGATCCTGGCTCAGGATGAACGCTGGCGGCGTGCTTAACACATGCA
AGTCGAACGGGGTGCTCATGACGGAGGATTCGTCCAACGGATTGAGTTACCTAGTG
GCGGACGGGTGAGTAACGCGTGAGGAACCTGCCTTGGAGAGGGGGATAACACTCCG
AAAGGAGTGCTAATACCGCATGATGCAGTTGGGTCGCATGGCTCTGACTGCCAAAG
ATTTATCGCTCTGAGATGGCCTCGCGTCTGATTAGCTAGTAGGTGGGGTAACGGCCC
ACCTAGGCGACGATCAGTAGCCGGACTGAGAGGTTGACCGGCCACATTGGGACTGA
GACACGGCCCAGACTCCTACGGGAGGCAGCAGTGGGGAATATTGGGCAATGGGCGC
AAGCCTGACCCAGCAACGCCGCGTGAAGGAAGAAGGCTTTCGGGTTGTAAACTTCT
TTTGTCAGGGACGAAACAAATGACGGTACCTGACGAATAAGCCACGGCTAACTACG
TGCCAGCAGCCGCGGTAATACGTAGGTGGCAAGCGTTATCCGGATTTACTGGGTGTA
AAGGGCGTGTAGGCGGGATTGCAAGTCAGATGTGAAAACTGGGGGCTCAACCTCCA
GCCTGCATTTGAAACTGTAGTTCTTGAGTGCTGGAGAGGCAATCGGAATTCCGTGTG
TAGCGGTGAAATGCGTAGATATACGGAGGAACACCAGTGGCGAAGGCGGATTGCTG
GACAGTAACTGACGCTGAGGCGCGAAAGCGTGGGGAGCAAACAGGATTAGATACCC
TGGTAGTCCACGCCGTAAACGATGGATACTAGGTGTGGGGGGTCTGACCCCCTCCGT
GCCGCAGTTAACACAATAAGTATCCCACCTGGGGAGTACGATCGCAAGGTTGAAAC
TCAAAGGAATTGACGGGGGCCCGCACAAGCGGTGGAGTATGTGGTTTAATTCGAAG
CAACGCGAAGAACCTTACCAGGGCTTGACATCCCACTAACGAAGCAGAGATGCATT
AGGTGCCCTTCGGGGAAAGTGGAGACAGGTGGTGCATGGTTGTCGTCAGCTCGTGTC
GTGAGATGTTGGGTTAAGTCCCGCAACGAGCGCAACCCTTATTGTTAGTTGCTACGC
AAGAGCACTCTAGCGAGACTGCCGTTGACAAAACGGAGGAAGGTGGGGACGACGTC
AAATCATCATGCCCCTTATGTCCTGGGCCACACACGTACTACAATGGTGGTTAACAG
AGGGAGGCAAAACCGCGAGGTGGAGCAAATCCCTAAAAGCCATCCCAGTTCGGATT
GCAGGCTGAAACCCGCCTGTATGAAGTTGGAATCGCTAGTAATCGCGGATCAGCAT
GCCGCGGTGAATACGTTCCCGGGCCTTGTACACACCGCCCGTCACACCATGAGAGTC
GGGAACACCCGAAGTCCGTAGCCTAACCG
NB2-A3NA Dorea longicatena
(SEQ ID NO: 22)
GCATGGTACAGTGGTAAAAACTCCGGTGGTATGAGATGGACCCGCGTCTGATTAGG
TAGTTGGTGGGGTAACGGCCTACCAAGCCGACGATCAGTAGCCGACCTGAGAGGGT
GACCGGCCACATTGGGACTGAGACACGGCCCAGACTCCTACGGGAGGCAGCAGTGG
GGAATATTGCACAATGGAGGAAACTCTGATGCAGCGACGCCGCGTGAAGGATGAAG
TATTTCGGTATGTAAACTTCTATCAGCAGGGAAGAAAATGACGGTACCTGACTAAGA
AGCCCCGGCTAACTACGTGCCAGCAGCCGCGGTAATACGTAGGGGGCAAGCGTTAT
CCGGATTTACTGGGTGTAAAGGGAGCGTAGACGGCACGGCAAGCCAGATGTGAAAG
CCCGGGGCTCAACCCCGGGACTGCATTTGGAACTGCTGAGCTAGAGTGTCGGAGAG
GCAAGTGGAATTNCTAGTGTAGCGGTGAAATGCGTAGATATTAGGAGGAACACCAG
TGGCGAAGGCGGCTTGCTGGACGATGACTGACGTTGAGGCTCGAAAGCGTGGGGAG
CAAACAGGATTAGATACCCTGGTAGTCCACGCCGTAAACGATGACTGCTAGGTGTC
GGGTGGCAAAGCCATTCGGTGCCGCAGCTAACGCAATAAGCAGTCCACCTGGGGAG
TACGTTCGCAAGAATGAAACTCAAAGGAATTGACGGGGACCCGCACAAGCGGTGGA
GCATGTGGTTTAATTCGAAGCAACGCGAAGAACCTTACCTGATCTTGACATCCCGAT
GACCGCTTCGTAATGGAAGNTTTTCTTCGGAACATCGGTGACAGGTGGTGCATGGTT
GTCGTCAGCTCGTGTCGTGAGATGTTGGGTTAAGTCCCGCAACGAGCGCAACCCCTA
TCTTCAGTAGCCAGCAGGTTAAGCTGGGCACTCTGGAGAGACTGCCAGGGATAACC
TGGAGGAAGGTGGGGATGACGTCAAATCATCATGCCCCTTATNACCAGGGCTACAC
ACGTGCTACAATGGCGTAAACAAAGAGACGCGAACTCGCGAGGGTAAGCAAATCTC
AAAAATAACGTCTCAGTTCGGATTGTAGTCTGCAACTCGACTACATGAAGCTGGAAT
CGCTAGTAATCGCAGATCAGAATGCTGCGGTGAATACGTTCCCGGGTCTTGTACACA
CCGCCCGTCACACCATGGGAGTCAGTAACGCCCGAAGTCAGTGACCCAACCGTAAG
GAGGGAGCTGCCGAAGGTGGGACCGATAACTGGGGTGAAGTCGTAACAAGGTAGCC
GTATCGGAAGGTGCGGCTGGATCACCTCCTTT
NB2-A5TSAB Blautia stercoris
(SEQ ID NO: 23)
TTCGCTTCCCTCTGTTTACGCCATTGTAGCACGTGTGTAGCCCAAATCATAAGGGGC
ATGATGATTTGACGTCATCCCCACCTTCCTCCAGGTTATCCCTGGCAGTCTCCTCAGA
GTGCCCACCATTACATGCTGGCTACTGGGGATAGGGGTTGCGCTCGTTGCGGGACTT
AACCCAACATCTCACGACACGAGCTGACGACAACCATGCACCACCTGTCTTGCCTGT
CCCGAAGGAAAGGTGACGTTACTCACCGGTCAGGCAGATGTCAAGACTTGGTAAGG
TTCTTCGCGTTGCTTCGAATTAAACCACATGCTCCACCGCTTGTGCGGGTCCCCGTCA
ATTCCTTTGAGTTTCATTCTTGCGAACGTACTCCCCAGGTGGAATACTTAATGCGTTT
GCGGCGGCACCGAAGAGCTGTGCTCCCCGACACCTAGTATTCATCGTTTACGGCGTG
GACTACCAGGGTATCTAATCCTGTTTGCTCCCCACGCTTTCGAGCCTCAACGTCAGTT
ACCGTCCAGTAAGCCGCCTTCGCCACTGGTGTTCCTCCTAATATCTACGCATTTCACC
GCTACACTAGGAATTCCGCTTACCCCTCCGGCACTCAAGCTTAACAGTTTCCAATGC
AGTCCCGGGGTTAAGCCCCAGCCTTTCACATCAGACTTGTTATGCCGTCTACGCTCC
CTTTACACCCAGTAAATCCGGATAACGCTTGCCCCCTACGTATTACCGCGGCTGCTG
GCACGTAGTTAGCCGGGGCTTCTTAGTCAGGTACCGTCATTTTCTTCCCTGCTGATAG
AAGTTTACATACCGAGATACTTCTTCCTTCACGCGGCGTCGCTGCATCAGGGTTTCCC
CCATTGTGCAATATTCCCCACTGCTGCCTCCCGTAGGAGTCTGGGCCGTGTCTCAGT
CCCAATGTGGCCGTTCACCCTCTCAGGCCGGCTATGGATCGTCGCTTTGGTAGGCCG
TTACCCTGCCAACTGGCTAATCCAACGCGGGTCCATCTTATACCACCTCAGTTTTTCA
CACCGGGCCATGCGGCCCTGTGCGCTTATGCGGTATTAGCAGCCATTTCTGACTGTT
ATCCCCCTGTATAAGGCAGGTTACCCACGCGTTACTCACCCGTCCGCCACTAGGATT
AAATCAAATCTGCCGAAGCTTCAATAAAATAATCCCCGTTCGACTTGCATGTGTTAA
GCACGCCGCCAGCGTTCATCCTGAGCCAGGATCAAACTCTCTGATAAAGTTTGATGT
CTCAAGACAACCAACTAGCTTAGTTATCTCTCGTCATTACTGTTTTAAAGTTCATTCT
TCCGAATGTGATTGTAAAAGAATTTTCGAGAATCGTATGTGTTTCACTGTTTAGTTAT
CAATGTTCATTGCTTTTTACTGTCTCTCGACAGCTTATTTACTTTACCACATCTTTTTT
TGTTTGTCAACAACTTTTTTGAAGTTTTTCAAACTTTTTTTCCGAAGATGAAGTTTTGT
CATCCGTGTTGACGACTTGACTACTTTATCATAGATAAGTCAGTTTGTCAACAGG
NB2-B11FAA Bifidobacterium longum
(SEQ ID NO: 24)
GTGGAGGGTTCGATTCTGGCTCAGGATGAACGCTGGCGGCGTGCTTAACACATGCA
AGTCGAACGGGATCCATCAGGCTTTGCTTGGTGGTGAGAGTGGCGAACGGGTGAGT
AATGCGTGACCGACCTGCCCCATACACCGGAATAGCTCCTGGAAACGGGTGGTAAT
GCCGGATGCTCCAGTTGATCGCATGGTCTTCTGGGAAAGCTTTCGCGGTATGGGATG
GGGTCGCGTCCTATCAGCTTGACGGCGGGGTAACGGCCCACCGTGGCTTCGACGGG
TAGCCGGCCTGAGAGGGCGACCGGCCACATTGGGACTGAGATACGGCCCAGACTCC
TACGGGAGGCAGCAGTGGGGAATATTGCACAATGGGCGCAAGCCTGATGCAGCGAC
GCCGCGTGAGGGATGGAGGCCTTCGGGTTGTAAACCTCTTTTATCGGGGAGCAAGC
GAGAGTGAGTTTACCCGTTGAATAAGCACCGGCTAACTACGTGCCAGCAGCCGCGG
TAATACGTAGGGTGCAAGCGTTATCCGGAATTATTGGGCGTAAAGGGCTCGTAGGC
GGTTCGTCGCGTCCGGTGTGAAAGTCCATCGCTTAACGGTGGATCCGCGCCGGGTAC
GGGCGGGCTTGAGTGCGGTAGGGGAGACTGGAATTCCCGGTGTAACGGTGGAATGT
GTAGATATCGGGAAGAACACCAATGGCGAAGGCAGGTCTCTGGGCCGTTACTGACG
CTGAGGAGCGAAAGCGTGGGGAGCGAACAGGATTAGATACCCTGGTAGTCCACGCC
GTAAACGGTGGATGCTGGATGTGGGGCCCGTTCCACGGGTTCCGTGTCGGAGCTAAC
GCGTTAAGCATCCCGCCTGGGGAGTACGGCCGCAAGGCTAAAACTCAAAGAAATTG
ACGGGGGCCCGCACAAGCGGCGGAGCATGCGGATTAATTCGATGCAACGCGAAGA
ACCTTACCTGGGCTTGACATGTTCCCGACGGTCGTAGAGATACGGCTTCCCTTCGGG
GCGGGTTCACAGGTGGTGCATGGTCGTCGTCAGCTCGTGTCGTGAGATGTTGGGTTA
AGTCCCGCAACGAGCGCAACCCTCGCCCCGTGTTGCCAGCGGATTATGCCGGGAAC
TCACGGGGGACCGCCGGGGTTAACTCGGAGGAAGGTGGGGATGACGTCAGATCATC
ATGCCCCTTACGTCCAGGGCTTCACGCATGCTACAATGGCCGGTACAACGGGATGCG
ACGCGGCGACGCGGAGCGGATCCCTGAAAACCGGTCTCAGTTCGGATCGCAGTCTG
CAACTCGACTGCGTGAAGGCGGAGTCGCTAGTAATCGCGAATCAGCAACGTCGCGG
TGAATGCGTTCCCGGGCCTTGTACACACCGCCCGTCAAGTCATGAAAGTGGGCAGC
ACCCGAAGCCGGTGGCCTAACCCCTTGTGGGATGGAGCCGTCTAAGGTGAGGCTCG
TGATTGGGACTAAGTCGTAACAAGGTAGCCGTACCGGAAGGTGCGGCTGGATCACC
TCCTTT
NB2-A2FAA Coprococcus comes
(SEQ ID NO: 25)
GAGAGTTTGATCCTGGCTCAGGATGAACGCTGGCGGCGTGCTTAACACATGCAAGTC
GAACGAAGCACCTGGATTTGATTCTTCGGATGAAGATCCTTGTGACTGAGTGGCGGA
CGGGTGAGTAACGCGTGGGTAACCTGCCTCATACAGGGGGATAACAGTTAGAAATG
ACTGCTAATACCGCATAAGACCACAGGGTCGCATGACCTGGTGGGAAAAACTCCGG
TGGTATGAGATGGACCCGCGTCTGATTAGGTAGTTGGTGGGGTAACGGCCTACCAA
GCCGACGATCAGTAGCCGACCTGAGAGGGTGACCGGCCACATTGGGACTGAGACAC
GGCCCAAACTCCTACGGGAGGCAGCAGTGGGGAATATTGCACAATGGGGGAAACCC
TGATGCAGCGACGCCGCGTGAGCGAAGAAGTATTTCGGTATGTAAAGCTCTATCAG
CAGGGAAGAAAATGACGGTACCTGACTAAGAAGCACCGGCTAAATACGTGCCAGCA
GCCGCGGTAATACGTATGGTGCAAGCGTTATCCGGATTTACTGGGTGTAAAGGGAG
CGTAGACGGCTGTGTAAGTCTGAAGTGAAAGCCCGGGGCTCAACCCCGGGACTGCT
TTGGAAACTATGCAGCTAGAGTGTCGGAGAGGTAAGTGGAATTCCCAGTGTAGCGG
TGAAATGCGTAGATATTGGGAGGAACACCAGTGGCGAAGGCGGCTTACTGGACGAT
GACTGACGTTGAGGCTCGAAAGCGTGGGGAGCAAACAGGATTAGATACCCTGGTAG
TCCACGCCGTAAACGATGACTACTAGGTGTCGGGGAGCAAAGCTCTTCGGTGCCGC
AGCAAACGCAATAAGTAGTCCACCTGGGGAGTACGTTCGCAAGAATGAAACTCAAA
GGAATTGACGGGGACCCGCACAAGCGGTGGAGCATGTGGTTTAATTCGAAGCAACG
CGAAGAACCTTACCTGCTCTTGACATCCCGGTGACCGGCATGTAATGATGCCTTTTC
TTCGGAACACCGGTGACAGGTGGTGCATGGTTGTCGTCAGCTCGTGTCGTGAGATGT
TGGGTTAAGTCCCGCAACGAGCGCAACCCTTATCTTCAGTAGCCAGCATTTCGGGTG
GGCACTCTGGAGAGACTGCCAGGGATAACCTGGAGGAAGGTGGGGATGACGTCAAA
TCATCATGCCCCTTATGAGCAGGGCTACACACGTGCTACAATGGCGTAAACAAAGG
GAAGCGAACCTGTGAGGGTAAGCAAATCTCAAAAATAACGTCTCAGTTCGGATTGT
AGTCTGCAACTCGACTACATGAAGCTGGAATCGCTAGTAATCGCGAATCAGCATGTC
GCGGTGAATACGTTCCCGGGTCTTGTACACACCGCCCGTCACACCATGGGAGTTGGT
AACGCCCGAAGTCAGTGACTCAACCGTAAGGAGAGAGCTGCCGAAGGTGGGACCG
ATAACTGGGGTGAAGTCGTAACAAGGTAGCCGTATCGGAAGGTGCGGCTGGATCAC
CTCCTTT
NB2-B6CNA [Eubacterium] eligens
(SEQ ID NO: 26)
TTCCTTAGAAAGGAGGTGATCCAGCCGCACCTTCCGATACGGCTACCTTGTTACGAC
TTCACCCCAGTTATCAAACCTGCCTTCGGCGGCTCCTTCTTTCGTTAGGTCACCGACT
TCGGGCATTTTCGACTCCCATGGTGTGACGGGCGGTGTGTACAAGACCCGGGAACGT
ATTCACCGCAGCATTCTGATCTGCGATTACTAGCGATTCCAGCTTCATGTAGTCGAG
TTGCAGACTACAATCCGAACTGAGACGTTATTTTTGTGATTTGCTTGGCCTCACGACT
TCGCTTCACTTTGTTTACGCCATTGTAGCACGTGTGTAGCCCAAGTCATAAGGGGCA
TGATGATTTGACGTCATCCCCACCTTCCTCCAGGTTATCCCTGGCAGTCTCCCTAGAG
TGCCCATCTTACTGCTGGCTACTAAGGATAGGGGTTGCGCTCGTTGCGGGACTTAAC
CCAACATCTCACGACACGAGCTGACGACAACCATGCACCACCTGTCTCCACTGTCCC
GAAGGAAAGGACACATTACTGTCCGGTCAGTGGGATGTCAAGACTTGGTAAGGTTC
TTCGCGTTGCTTCGAATTAAACCACATGCTCCACCGCTTGTGCGGGTCCCCGTCAATT
CCTTTGAGTTTCATTCTTGCGAACGTACTCCCCAGGTGGAATACTTATTGCGTTTGCT
GCGGCACCGAAGCCCTTATGGGCCCCGACACCTAGTATTCATCGTTTACGGCGTGGA
CTACCAGGGTATCTAATCCTGTTTGCTCCCCACGCTTTCGAGCCTCAGTGTCAGTTAC
AGTCCAGTGAGCCGCCTTCGCCACTGGTGTTCCTCCTAATATCTACGCATTTCACCGC
TACACTAGGAATTCCACTCACCCCTCCTGCACTCCAGCCTTACAGTTTCAAAAGCAG
TTCCGGGGTTGAGCCCCGGATTTTCACTTCTGACTTGCATGGCCACCTACACTCCCTT
TACACCCAGTAAATCCGGATAACGCTTGCTCCATACGTATTACCGCGGCTGCTGGCA
CGTATTTAGCCGGAGCTTCTTAGTCAGGTACCGTCACTATCTTCCCTGCTGATAGAGC
TTTACATAACGAATTACTTCTTCACTCACGCGGCGTCGCTGCATCAGAGTTTCCTCCA
TTGTGCAATATTCCCCACTGCTGCCTCCCGTAGGAGTCTGGGCCGTGTCTCAGTCCC
AATGTGGCCGGTCACCCTCTCAGGTCGGCTACTGATCGTCGCCTTGGTGGGCTGTTA
TCTCACCAACTAGCTAATCAGACGCGGGTCCATCTTATACCACCGGAGTTTTTCACA
CCATGTCATGCAACATTGTGCGCTTATGCGGTATTACCAGCCGTTTCCAGCTGCTATC
CCCCAGTACAAGGCAGGTTACCCACGCGTTACTCACCCGTCCGCCACTCAGTCATAA
AGAACTTCAAACCGAAGTAATCCGTTCTAAATGCTTCGTTCGACTTGCATGTGTTAA
GCACGCCGCCAGCGTTCATCCTGAGCCAGGATCAAACTCTCATA
NB2-BAERMRS02 Lactobacillus paracasei
(SEQ ID NO: 27)
CCAAGGCGATGATACGTAGCCGAACTGAGAGGTTGATCGGCCACATTGGGACTGAG
ACACGGCCCAAACTCCTACGGGAGGCAGCAGTAGGGAATCTTCCACAATGGACGCA
AGTCTGATGGAGCAACGCCGCGTGAGTGAAGAAGGCTTTCGGGTCGTAAAACTCTG
TTGTTGGAGAAGAATGGTCGGCAGAGTAACTGTTGNCGNCGTGACGGTATCCAACC
AGAAAGCCACGGCTAACTACGTGCCAGCAGCCGCGGTAATACGTAGGTGGCAAGCG
TTATCCGGATTTATTGGGCGTAAAGCGAGCGCAGGCGGTTTTTTAAGTCTGATGTGA
AAGCCCTCGGCTTAACCGAGGAAGCGCATCGGAAACTGGGAAACTTGAGTGCAGAA
GAGGACAGTGGAACTCCATGTGTAGCGGTGAAATGCGTAGATATATGGAAGAACAC
CAGTGGCGAAGGCGGCTGTCTGGTCTGTAACTGACGCTGAGGCTCGAAAGCATGGG
TAGCGAACAGGATTAGATACCCTGGTAGTCCATGCCGTAAACGATGAATGCTAGGT
GTTGGAGGGTTTCCGCCCTTCAGTGCCGCAGCTAACGCATTAAGCATTCCGCCTGGG
GAGTACGACCGCAAGGTTGAAACTCAAAGGAATTGACGGGGGCCCGCACAAGCGGT
GGAGCATGTGGTTTAATTCGAAGCAACGCGAAGAACCTTACCAGGTCTTGACATCTT
TTGATCACCTGAGAGATCAGGTTTCCCCTTCGGGGGCAAAATGACAGGTGGTGCATG
GTTGTCGTCAGCTCGTGTCGTGAGATGTTGGGTTAAGTCCCGCAACGAGCGCAACCC
TTATGACTAGTTGCCAGCATTTAGTTGGGCACTCTAGTAAGACTGCCGGTGACAAAC
CGGAGGAAGGTGGGGATGACGTCAAATCATCATGCCCCTTATGACCTGGGCTACAC
ACGTGCTACAATGGATGGTACAACGAGTTGCGAGACCGCGAGGTCAAGCTAATCTC
TTAAAGCCATTCTCAGTTCGGACTGTAGGCTGCAACTCGCCTACACGAAGTCGGAAT
CGCTAGTAATCGCGGATCAGCACGCCGCGGTGAATACGTTCCCGGGCCTTGTACACA
CCGCCCGTCACACCATGAGAGTTTGTAACACCCGAAGCCGGTGGCGTAACCCTTTTA
GGGAGCGAGCCGTCTAAGGTGGGACAAATGATTAGGGTGAAGTCGTAACAAGGTAG
CCGTAGGAGAACCTGCGGCTGGATCACCTCCTTT
NB2-B13CNA [Clostridium] oroticum
(SEQ ID NO: 28)
CCTTAGAAAGGAGGTGATCCAGCCGCACCTTCCGATACGGCTACCTTGTTACGACTT
CACCCCAGTTATCGGTCCCACCTTCGGCAGCTCCCTCCTTGCGGTTGGGTCACTGACT
TCGGGCGTTACCAACTCCCATGGTGTGACGGGCGGTGTGTACAAGACCCGGGAACG
TATTCACCGCGACATTCTGATTCGCGATTACTAGCGATTCCAGCTTCATGTAGTCGA
GTTGCAGACTACAATCCGAACTGAGACGTTATTTTTGAGATTTGCTTACCCTCGCAG
GCTCGCTTCCCTTTGTTTACGCCATTGTAGCACGTGTGTAGCCCTGCTCATAAGGGGC
ATGATGATTTGACGTCATCCCCACCTTCCTCCAGGTTATCCCTGGCAGTCTCTCTAGA
GTGCCCGGCCRWACCGCTGGCTACTAAAGATAGGGGTTGCGCTCGTTGCGGGACTT
AACCCAACATCTCACGACACGAGCTGACGACAACCATGCACCACCTGTCATCCCTGT
CCCGAAGGAAAGGCAACATTACTTGCCGGTCAGGGAGATGTCAAGAGCAGGTAAGG
TTCTTCGCGTTGCTTCGAATTAAACCACATGCTCCACCGCTTGTGCGGGTCCCCGTCA
ATTCCTTTGAGTTTCATTCTTGCGAACGTACTCCCCAGGTGGACTACTTATTGCGTTG
GCTGCGGCACCGAATAGCTCTGCTACCCGACACCTAGTAGTCATCGTTTACGGCGTG
GACTACCAGGGTATCTAATCCTGTTTGCTCCCCACGCTTTCGAGCCTCAACGTCAGT
CATCGTCCAGCAAGCCGCCTTCGCCACTGGTGTTCCTCCTAATATCTACGCATTTCAC
CGCTACACTAGGAATTCCACTTGCCTCTCCGACACTCTAGTTCGACAGTTTCCAATG
CAGTCCCAGGGTTGAGCCCTGGCCTTTCACATCAGACTTGCCATACCGTCTACGCTC
CCTTTACACCCAGTAAATCCGGATAACGCTTGCCCCCTACGTATTACCGCGGCTGCT
GGCACGTAGTTAGCCGGGGCTTCTTAGTCAGGTACCGTCATTTTCTTCCCTGCTGATA
GAAGTTTACATACCGAAATACTTCATCCTTCACGCGGCGTCGCTGCATCAGGGTTTC
CCCCATTGTGCAATATTCCCCACTGCTGCCTCCCGTAGGAGTTTGGGCCGTGTCTCA
GTCCCAATGTGGCCGGTCACCCTCTCAGGTCGGCTACTGATCGTCGCCTTGGTAGGC
CGTTACCCCACCAACYAGCTAATCAGACGCGGGTCCATCTCATACCACCGGAGTTTT
TACCCCTGCACCATGCGGTGCTGTGGTCTTATGCGGTATTAGCAGYCATTTCTAACT
GTTATCCCCCTGTATGAGGCAGGTTACCCACGCGTTACTCACCCGTCCGCCACTCAG
TCACAAAAGTCTTCATCCGAAGAATCAAACTTAAGTGCTTCGTTCGACTTGCATGTG
TTAAGCACGCCGCCAGCGTTCATCCTGAGCCAGGATCAAACTCTCGT
NB2-B15DCM Dorea formicigenerans
(SEQ ID NO: 29)
TTAAACGAGAGTTTGATCCTGGCTCAGGATGAACGCTGGCGGCGTGCTTAACACATG
CAAGTCGAGCGAAGCACTTAAGTTCGATTCTTCGGATGAAGACTTTTGTGACTGAGC
GGCGGACGGGTGAGTAACGCGTGGGTAACCTGCCTCATACAGGGGGATAACAGTTA
GAAATGGCTGCTAATACCGCATAAGACCACAGTACTGCATGGTACAGTGGTAAAAA
CTCCGGTGGTATGAGATGGACCCGCGTCTGATTAGGTAGTTGGTGAGGTAACGGCCC
ACCAAGCCGACGATCAGTAGCCGACCTGAGAGGGTGACCGGCCACATTGGGACTGA
GACACGGCCCAGACTCCTACGGGAGGCAGCAGTGGGGAATATTGCACAATGGGCGA
AAGCCTGATGCAGCGACGCCGCGTGAAGGATGAAGTATTTCGGTATGTAAACTTCTA
TCAGCAGGGAAGAAAATGACGGTACCTGACTAAGAAGCCCCGGCTAACTACGTGCC
AGCAGCCGCGGTAATACGTAGGGGGCAAGCGTTATCCGGATTTACTGGGTGTAAAG
GGAGCGTAGACGGCTGTGCAAGTCTGAAGTGAAAGGCATGGGCTCAACCTGTGGAC
TGCTTTGGAAACTGTGCAGCTAGAGTGTCGGAGAGGTAAGTGGAATTCCTAGTGTAG
CGGTGAAATGCGTAGATATTAGGAGGAACACCAGTGGCGAAGGCGGCTTACTGGAC
GATGACTGACGTTGAGGCTCGAAAGCGTGGGGAGCAAACAGGATTAGATACCCTGG
TAGTCCACGCCGTAAACGATGACTGCTAGGTGTCGGGTAGCAAAGCTATTCGGTGCC
GCAGCTAACGCAATAAGCAGTCCACCTGGGGAGTACGTTCGCAAGAATGAAACTCA
AAGGAATTGACGGGGACCCGCACAAGCGGTGGAGCATGTGGTTTAATTCGAAGCAA
CGCGAAGAACCTTACCTGATCTTGACATCCCGATGACCGCTTCGTAATGGAAGCTTT
TCTTCGGAACATCGGTGACAGGTGGTGCATGGTTGTCGTCAGCTCGTGTCGTGAGAT
GTTGGGTTAAGTCCCGCAACGAGCGCAACCCTTATCTTCAGTAGCCAGCATTTAAGA
TGGGCACTCTGGAGAGACTGCCAGGGATAACCTGGAGGAAGGTGGGGATGACGTCA
AATCATCATGCCCCTTATGACCAGGGCTACACACGTGCTACAATGGCGTAAACAAA
GGGAAGCAGAGCCGCGAGGCCGAGCAAATCTCAAAAATAACGTCTCAGTTCGGATT
GTAGTCTGCAACTCGACTACATGAAGCTGGAATCGCTAGTAATCGCAGATCAGAAT
GCTGCGGTGAATACGTTCCCGGGTCTTGTACACACCGCCCGTCACACCATGGGAGTC
AGTAACGCCCGAAGTCAGTGACCCAACCGAAAGGAGGGAGCTGCCGAAGGTGGGA
CCGATAACTGGGGTGAAGTCGTAACAAGGTAGCCGTATCGGAAGGTGCGGCTGGAT
CACCTCCTTTCT
NB2-BBHI1 Escherichia coli
(SEQ ID NO: 30)
ACGAGTGGCGGACGGGTGAGTAATGTCTGGGAAACTGCCTGATGGAGGGGGATAAC
TACTGGAAACGGTAGCTAATACCGCATAACGTCGCAAGACCAAAGAGGGGGACCTT
CGGGCCTCTTGCCATCGGATGTGCCCAGATGGGATTAGCTAGTAGGTGGGGTAACG
GCTCACCTAGGCGACGATCCCTAGCTGGTCTGAGAGGATGACCAGCCACACTGGAA
CTGAGACACGGTCCAGACTCCTACGGGAGGCAGCAGTGGGGAATATTGCACAATGG
GCGCAAGCCTGATGCAGCCATGCCGCGTGTATGAAGAAGGCCTTCGGGTTGTAAAG
TACTTTCAGCGGGGAGGAAGGGAGTAAAGTTAATACCTTTNCTCATTGACGTTACCC
GCAGAAGAAGCACCGGCTAACTCCGTGCCAGCAGCCGCGGTAATACGGAGGGTGCA
AGCGTTAATCGGAATTACTGGGCGTAAAGCGCACGCAGGCGGTTTGTTAAGTCAGA
TGTGAAATCCCCGGGCTCAACCTGGGAACTGCATCTGATACTGGCAAGCTTGAGTCT
CGTAGAGGGGGGTAGAATTCCAGGTGTAGCGGTGAAATGCGTAGAGATCTGGAGGA
ATACCGGTGGCGAAGGCGGCCCCCTGGACGAAGACTGACGCTCAGGTGCGAAAGCG
TGGGGAGCAAACAGGATTAGATACCCTGGTAGTCCACGCCGTAAACGATGTCGACT
TGGAGGTTGTGCCCTTGAGGCGTGGCTTCCGGAGCTAACGCGTTAAGTCGACCGCCT
GGGGAGTACGGCCGCAAGGTTAAAACTCAAATGAATTGACGGGGGCCCGCACAAGC
GGTGGAGCATGTGGTTTAATTCGATGCAACGCGAAGAACCTTACCTGGTCTTGACAT
CCACNGAANTTTNCAGAGATGAGAATGTGCCTTCGGGAACNGTGAGACAGGTGCTG
CATGGCTGTCGTCAGCTCGTGTTGTGAAATGTTGGGTTAAGTCCCGCAACGAGCGCA
ACCCTTATCCTTTGTTGCCAGCGGTCCGGCCGGGAACTCAAAGGAGACTGCCAGTGA
TAAACTGGAGGAAGGTGGGGATGACGTCAAGTCATCATGGCCCTTACGACCAGGGC
TACACACGTGCTACAATGGCGCATACAAAGAGAAGCGACCTCGCGAGAGCAAGCGG
ACCTCATAAAGTGCGTCGTAGTCCGGATTGGAGTCTGCAACTCGACTCCATGAAGTC
GGAATCGCTAGTAATCGTGGATCAGAATGCCACGGTGAATACGTTCCCGGGCCTTGT
ACACACCGCCCGTCACACCATGGGAGTGGGTTGCAAAAGAAGTAGGTAGCTTAACC
TTCGGGAGGGCGCTTACCACTTTGTGATTCATGACTGGGGTGAAGTCGTAACAAGGT
AACCGTAGGGGAACCTGCGGTTGGATCACCTCCTT
NB2-B9DCM Anaerostipes hadrus
(SEQ ID NO: 31)
ATGAGAGTTTGATCCTGGCTCAGGATGAACGCTGGCGGCGTGCTTAACACATGCAA
GTCGAACGAAGCGCCTTATTTGATTTTCTTCGGAACTGAAGATTTGGTGACTGAGTG
GCGGACGGGTGAGTAACGCGTGGGTAACCTGCCCTGTACAGGGGGATAACAATCAG
AAATGACTGCTAATACCGCATAAGACCACAGCACCGCATGGTGCAGGGGTAAAAAC
TCCGGTGGTACAGGATGGACCCGCGTCTGATTAGCTGGTTGGTGAGGTAACGGCTCA
CCAAGGCGACGATCAGTAGCCGGCTTGAGAGAGTGAACGGCCACATTGGGACTGAG
ACACGGCCCAAACTCCTACGGGAGGCAGCAGTGGGGAATATTGCACAATGGGGGGA
ACCCTGATGCAGCGACGCCGCGTGAGTGAAGAAGTATTTCGGTATGTAAAGCTCTAT
CAGCAGGGAAGAAAATGACGGTACCTGACTAAGAAGCCCCGGCTAACTACGTGCCA
GCAGCCGCGGTAATACGTAGGGGGCAAGCGTTATCCGGAATTACTGGGTGTAAAGG
GTGCGTAGGTGGTATGGCAAGTCAGAAGTGAAAACCCAGGGCTTAACTCTGGGACT
GCTTTTGAAACTGTCAGACTGGAGTGCAGGAGAGGTAAGCGGAATTCCTAGTGTAG
CGGTGAAATGCGTAGATATTAGGAGGAACATCAGTGGCGAAGGCGGCTTACTGGAC
TGAAACTGACACTGAGGCACGAAAGCGTGGGGAGCAAACAGGATTAGATACCCTGG
TAGTCCACGCCGTAAACGATGAATACTAGGTGTCGGGGCCGTAGAGGCTTCGGTGC
CGCAGCCAACGCAATAAGTATTCCACCTGGGGAGTACGTTCGCAAGAATGAAACTC
AAAGGAATTGACGGGGACCCGCACAAGCGGTGGAGCATGTGGTTTAATTCGAAGCA
ACGCGAAGAACCTTACCTGGTCTTGACATCCTTCTGACCGGTCCTTAACCGGACCTT
TCCTTCGGGACAGGAGTGACAGGTGGTGCATGGTTGTCGTCAGCTCGTGTCGTGAGA
TGTTGGGTTAAGTCCCGCAACGAGCGCAACCCCTATCTTTAGTAGCCAGCATTTAAG
GTGGGCACTCTAGAGAGACTGCCAGGGATAACCTGGAGGAAGGTGGGGACGACGTC
AAATCATCATGCCCCTTATGACCAGGGCTACACACGTGCTACAATGGCGTAAACAG
AGGGAAGCAGCCTCGTGAGAGTGAGCAAATCCCAAAAATAACGTCTCAGTTCGGAT
TGTAGTCTGCAACTCGACTACATGAAGCTGGAATCGCTAGTAATCGCGAATCAGAAT
GTCGCGGTGAATACGTTCCCGGGTCTTGTACACACCGCCCGTCACACCATGGGAGTC
AGTAACGCCCGAAGTCAGTGACCCAACCGTAAGGAGGGAGCTGCCGAAGGCGGGA
CCGATAACTGGGGTGAAGTCGTAACAAGGTAGCCGTATCGGAAGGTGCGGCTGGAT
CACCTCCTTT
NB2-B9FAA Blautia luti
(SEQ ID NO: 32)
GTTGGTGGGGTAACGGCCCACCAAGGCGACGATCCATAGCCGGCCTGAGAGGGTGA
ACGGCCACATTGGGACTGAGACACGGCCCAGACTCCTACGGGAGGCAGCAGTGGGG
AATATTGCACAATGGGGGAAACCCTGATGCAGCGACGCCGCGTGAAGGAAGAAGTA
TCTCGGTATGTAAACTTCTATCAGCAGGGAAGATAGTGACGGTACCTGACTAAGAA
GCCCCGGCTAACTACGTGCCAGCAGCCGCGGTAATACGTAGGGGGCAAGCGTTATC
CGGATTTACTGGGTGTAAAGGGAGCGTAGACGGTGTGGCAAGTCTGATGTGAAAGG
CATGGGCTCAACCTGTGGACTGCATTGGAAACTGTCATACTTGAGTGCCGGAGGGGT
AAGCGGAATTCCTAGTGTAGCGGTGAAATGCGTAGATATTAGGAGGAACACCAGTG
GCGAAGGCGGCTTACTGGACGGTAACTGACGTTGAGGCTCGAAAGCGTGGGGAGCA
AACAGGATTAGATACCCTGGTAGTCCACGCCGTAAACGATGAATACTAGGTGTCGG
GGAGCAAAGCTCTTCGGTGCCGTCGCAAACGCAGTAAGTATTCCACCTGGGGAGTA
CGTTCGCAAGAATGAAACTCAAAGGAATTGACGGGGACCCGCACAAGCGGTGGAGC
ATGTGGTTTAATTCGAAGCAACGCGAAGAACCTTACCAAATCTTGACATCCCTCTGA
CCGGTCTTTAATCGGACCTTCTCTTCGGAGCAGAGGTGACAGGTGGTGCATGGTTGT
CGTCAGCTCGTGTCGTGAGATGTTGGGTTAAGTCCCGCAACGAGCGCAACCCCTATC
CTCAGTAGCCAGCATTTAAGGTGGGCACTCTGGGGAGACTGCCAGGGATAACCTGG
AGGAAGGCGGGGATGACGTCAAATCATCATGCCCCTTATGATTTGGGCTACACACGT
GCTACAATGGCGTAAACAAAGGGAAGCGAGATCGTGAGATGGAGCAAATCCCAAA
AATAACGTCCCAGTTCGGACTGTAGTCTGCAACCCGACTACACGAAGCTGGAATCG
CTAGTAATCGCGGATCAGAATGCCGCGGTGAATACGTTCCCGGGTCTTGTACACACC
GCCCGTCACACCATGGGAGTCAGTAACGCCCGAAGTCAGTGACCTAACTGCAAAGA
AGGAGCTGCCGAAGGCGGGACCGATGACTGGGGTGAAGTCGTAACAAGGTAGCCGT
ATCGGAAGGTGCGGCTGGATCAC
NB2-A7D5 [Clostridium] scindens
(SEQ ID NO: 33)
AACGAGAGTTTGATCCTGGCTCAGGATGAACGCTGGCGGCGTGCCTAACACATGCA
AGTCGAACGAAGCGCTTCCGCCTGATTTTCTTCGGAGATGAAGGCGGCTGCGACTGA
GTGGCGGACGGGTGAGTAACGCGTGGGCAACCTGCCTTGCACTGGGGGATAACAGC
CAGAAATGGCTGCTAATACCGCATAAGACCGAAGCGCCGCATGGCGCAGCGGCCAA
AGCCCCGGCGGTGCAAGATGGGCCCGCGTCTGATTAGGTAGTTGGCGGGGTAACGG
CCCACCAAGCCGACGATCAGTAGCCGACCTGAGAGGGTGACCGGCCACATTGGGAC
TGAGACACGGCCCAGACTCCTACGGGAGGCAGCAGTGGGGAATATTGCACAATGGG
GGAAACCCTGATGCAGCGACGCCGCGTGAAGGATGAAGTATTTCGGTATGTAAACT
TCTATCAGCAGGGAAGAAGATGACGGTACCTGACTAAGAAGCCCCGGCTAACTACG
TGCCAGCAGCCGCGGTAATACGTAGGGGGCAAGCGTTATCCGGATTTACTGGGTGT
AAAGGGAGCGTAGACGGCGATGCAAGCCAGATGTGAAAGCCCGGGGCTCAACCCC
GGGACTGCATTTGGAACTGCGTGGCTGGAGTGTCGGAGAGGCAGGCGGAATTCCTA
GTGTAGCGGTGAAATGCGTAGATATTAGGAGGAACACCAGTGGCGAAGGCGGCCTG
CTGGACGATGACTGACGTTGAGGCTCGAAAGCGTGGGGAGCAAACAGGATTAGATA
CCCTGGTAGTCCACGCCGTAAACGATGACTACTAGGTGTCGGGTGGCAAGGCCATTC
GGTGCCGCAGCAAACGCAATAAGTAGTCCACCTGGGGAGTACGTTCGCAAGAATGA
AACTCAAAGGAATTGACGGGGACCCGCACAAGCGGTGGAGCATGTGGTTTAATTCG
AAGCAACGCGAAGAACCTTACCTGATCTTGACATCCCGATGCCAAAGCGCGTAACG
CGCTCTTTCTTCGGAACATCGGTGACAGGTGGTGCATGGTTGTCGTCAGCTCGTGTC
GTGAGATGTTGGGTTAAGTCCCGCAACGAGCGCAACCCCTATCTTCAGTAGCCAGCA
TTCCGGATGGGCACTCTGGAGAGACTGCCAGGGACAACCTGGAGGAAGGTGGGGAT
GACGTCAAATCATCATGCCCCTTATGACCAGGGCTACACACGTGCTACAATGGCGTA
AACAAAGGGAGGCGAACCCGCGAGGGTGGGCAAATCCCAAAAATAACGTCTCAGTT
CGGATTGTAGTCTGCAACTCGACTACATGAAGCTGGAATCGCTAGTAATCGCGAATC
AGAATGTCGCGGTGAATACGTTCCCGGGTCTTGTACACACCGCCCGTCACACCATGG
GAGTCAGTAACGCCCGAAGCCGGTGACCCAACCCGCAAGGGAGGGAGCCGTCGAA
GGTGGGACCGATAACTGGGGTGAAGTCGTAACAAGGTAGCCGTATCGGAAGGTGCG
GCTGGATCACCTCCTTT
NB2-B10MRS Eubacterium desmolans
(SEQ ID NO: 34)
TTTAGAGAGTTTGATCCTGGCTCAGGATGAACGCTGGCGGCGTGCCTAACACATGCA
AGTCGAACGGAGTCGTTTTGGAAAATCCTTCGGGATTGGAATTCTCGACTTAGTGGC
GGACGGGTGAGTAACGCGTGAGCAATCTGCCTTTAAGAGGGGGATAACAGTCGGAA
ACGGCTGCTAATACCGCATAAAGCATTGAATTCGCATGTTTTCGATGCCAAAGGAGC
AATCCGCTTTTAGATGAGCTCGCGTCTGATTAGCTAGTTGGTGGGGTAACGGCCCAC
CAAGGCGACGATCAGTAGCCGGACTGAGAGGTTGAACGGCCACATTGGGACTGAGA
CACGGCCCAGACTCCTACGGGAGGCAGCAGTGGGGAATATTGCGCAATGGGGGAAA
CCCTGACGCAGCAACGCCGCGTGATTGAAGAAGGCCTTCGGGTTGTAAAGATCTTTA
ATCAGGGACGAAAAATGACGGTACCTGAAGAATAAGCTCCGGCTAACTACGTGCCA
GCAGCCGCGGTAATACGTAGGGAGCAAGCGTTATCCGGATTTACTGGGTGTAAAGG
GCGCGCAGGCGGGCCGGCAAGTTGGAAGTGAAATCCGGGGGCTTAACCCCCGAACT
GCTTTCAAAACTGCTGGTCTTGAGTGATGGAGAGGCAGGCGGAATTCCGTGTGTAGC
GGTGAAATGCGTAGATATACGGAGGAACACCAGTGGCGAAGGCGGCCTGCTGGACA
TTAACTGACGCTGAGGCGCGAAAGCGTGGGGAGCAAACAGGATTAGATACCCTGGT
AGTCCACGCCGTAAACGATGGATACTAGGTGTGGGAGGTATTGACCCCTTCCGTGCC
GCAGTTAACACAATAAGTATCCCACCTGGGGAGTACGGCCGCAAGGTTGAAACTCA
AAGGAATTGACGGGGGCCCGCACAAGCAGTGGAGTATGTGGTTTAATTCGAAGCAA
CGCGAAGAACCTTACCAGGCCTTGACATCCCGATGACCGGCTTAGAGATAAGCCTTC
TCTTCGGAGCATCGGTGACAGGTGGTGCATGGTTGTCGTCAGCTCGTGTCGTGAGAT
GTTGGGTTAAGTCCCGCAACGAGCGCAACCCTTACGGTTAGTTGATACGCAAGATCA
CTCTAGCCGGACTGCCGTTGACAAAACGGAGGAAGGTGGGGACGACGTCAAATCAT
CATGCCCCTTATGGCCTGGGCTACACACGTACTACAATGGCAGTCATACAGAGGGA
AGCAAAATCGCGAGGTGGAGCAAATCCCTAAAAGCTGTCCCAGTTCAGATTGCAGG
CTGCAACCCGCCTGCATGAAGTCGGAATTGCTAGTAATCGCGGATCAGCATGCCGC
GGTGAATACGTTCCCGGGCCTTGTACACACCGCCCGTCACACCATGAGAGCCGTCAA
TACCCGAAGTCCGTAGCCTAACCGCAAGGAGGGCGCGGCCGAAGGTAGGGGTGGTA
ATTAGGGTGAAGTCGTAACAAGGTAGCCGTATCGGAAGGTGCGGCTGGATCACCTC
CTTT
NB2-B19DCM Faecalibacterium prausnitzii
(SEQ ID NO: 35)
AGAAAGGAGGTGATCCAGCCGCAGGTTCTCCTACGGCTACCTTGTTACGACTTCACC
CCAATCACCAGTTTTACCTTCGGCGGCGTCCTCCTTGCGGTTAGACTACCGACTTCG
GGTCCCCCCGGCTCTCATGGTGTGACGGGCGGTGTGTACAAGGCCCGGGAACGTATT
CACCGTGGCATGCTGATCCACGATTACTAGCAATTCCGACTTCGTGCAGGCGAGTTG
CAGCCTGCAGTCCGAACTGGGACGTTGTTTCTGAGTTTTGCTCCACCTCGCGGTCTTG
CTTCTCTTTGTTTAACGCCATTGTAGTACGTGTGTAGCCCAAGTCATAAAGGGCATG
ATGATTTGACGTCATCCCCACCTTCCTCCGTTTTGTCAACGGCAGTCCTGCCAGAGTC
CTCTTGCGTAGTAACTGACAGTAAGGGTTGCGCTCGTTGCGGGACTTAACCCAACAT
CTCACGACACGAGCTGACGACAACCATGCACCACCTGTCTCCTTGCTCCGAAGAGA
AAACATATTTCTATGTGCGTCGCAGGATGTCAAGACTTGGTAAGGTTCTTCGCGTTG
CGTCGAATTAAACCACATACTCCACTGCTTGTGCGGGCCCCCGTCAATTCCTTTGAG
TTTCAACCTTGCGGTCGTACTCCCCAGGTGGATTACTTATTGTGTTAACTGCGGCACT
GAAGGGGTCAATCCTCCAACACCTAGTAATCATCGTTTACGGTGTGGACTACCAGGG
TATCTAATCCTGTTTGCTACCCACACTTTCGAGCCTCAGCGTCAGTTGGTGCCCAGTA
GGCCGCCTTCGCCACTGGTGTTCCTCCCGATATCTACGCATTCCACCGCTACACCGG
GAATTCCGCCTACCTCTGCACTACTCAAGAAAAACAGTTTTGAAAGCAGTTTATGGG
TTGAGCCCATAGATTTCACTTCCAACTTGTCTTCCCGCCTGCGCTCCCTTTACACCCA
GTAATTCCGGACAACGCTTGTGACCTACGTTTTACCGCGGCTGCTGGCACGTAGTTA
GCCGTCACTTCCTTGTTGAGTACCGTCATTATCTTCCTCAACAACAGGAGTTTACAAT
CCGAAGACCTTCTTCCTCCACGCGGCGTCGCTGCATCAGGGTTTCCCCCATTGTGCA
ATATTCCCCACTGCTGCCTCCCGTAGGAGTCTGGGCCGTGTCTCAGTCCCAATGTGG
CCGTTCAACCTCTCAGTCCGGCTACCGATCGTTGCCTTGGTGGGCCATTACCTCACC
AACTAGCTAATCGGACGCGAGGCCATCTCAAAGCGGATTGCTCCTTTTCCCTCTGGT
CGATGCCGACCTGTGGGCTTATGCGGTATTAGCAGTCGTTTCCAACTGTTGTCCCCCT
CTTTGAGGCAGGTTCCTCACGCGTTACTCACCCGTTCGCCACTCGCTYGAGAAAGCA
AGCTCTCTCTCGCTCGTTCGACTTGCATGTGTTAGGCGCGCCGCCAGCGTTCGTCCTG
AGCCAGGATCAAACTCTTTATAAA
NB2-Al2BBE Bacteroides ovatus
(SEQ ID NO: 36)
CCGTGTCTCAGTTCCAATGTGGGGGACCTTCCTCTCAGAACCCCTATCCATCGTTGTC
TTGGTGGGCCGTTACCCCGCCAACAAACTAATGGAACGCATCCCCATCGATAACCG
AAATTCTTTAATAGTAAAACCATGCGGTTTTAATATACCATCGGATATTAATCTTTCT
TTCGAAAGGCTATCCCCGAGTTATCGGCAGGTTGGATACGTGTTACTCACCCGTGCG
CCGGTCGCCATCTTTAGTTTGCAAGCAAACTAAAATGCTGCCCCTCGACTTGCATGT
GTTAAGCCTGTAGCTAGCGTTCATCCTGAGCTATTAAAGAATTTCGGTTATCGATGG
GGATGCGTTCCATTAGTTTGTTGGCGGGGTAACGGCCCACCAAGACAACGATGGAT
AGGGGTTCTGAGAGGAAGGTCCCCCACATTGGAACTGAGACACGGTCCAAACTCCT
ACGGGAGGCAGCAGTGAGGAATATTGGTCAATGGGCGAGAGCCTGAACCAGCCAA
GTAGCGTGAAGGATGAAGGCTCTATGGGTCGTAAACTTCTTTTATATGGGAATAAAG
TATTCCACGTGTGGAATTTTGTATGTACCATATGAATAAGGATCGGCTAACTCCGTG
CCAGCAGCCGCGGTAATACGGAGGATCCGAGCGTTATCCGGATTTATTGGGTTTAAA
GGGAGCGTAGGTGGATTGTTAAGTCAGTTGTGAAAGTTTGCGGCTCAACCGTAAAAT
TGCAGTTGAAACTGGCAGTCTTGAGTACAGTAGAGGTGGGCGGAATTCGTGGTGTA
GCGGTGAAATGCTTAGATATCACGAAGAACTCCGATTGCGAAGGCAGCTCACTAGA
CTGTCACTGACACTGATGCTCGAAAGTGTGGGTATCAAACAGGATTAGATACCCTGG
TAGTCCACACAGTAAACGATGAATACTCGCTGTTTGCGATATACAGTAAGCGGCCAA
GCGAAAGCATTAAGTATTCCACCTGGGGAGTACGCCGGCAACGGTGAAACTCAAAG
GAATTGACGGGGGCCCGCACAAGCGGAGGAACATGTGGTTTAATTCGATGATACGC
GAGGAACCTTACCCGGGCTTAAATTGCATTTGAATAATCTGGAAACAGGTTAGCCGC
AAGGCAAATGTGAAGGTGCTGCATGGTTGTCGTCAGCTCGTGCCGTGAGGTGTCGGC
TTAAGTGCCATAACGAGCGCAACCCTTATCTTTAGTTACTAACAGGTTATGCTGAGG
ACTCTAGAGAGACTGCCGTCGTAAGATGTGAGGAAGGTGGGGATGACGTCAAATCA
GCACGGCCCTTACGTCCGGGGCTACACACGTGTTACAATGGGGGGTACAGAAGGCA
GCTACCTGGCGACAGGATGCTAATCCCAAAAACCTCTCTCAGTTCGGATCGAAGTCT
GCAACCCGACTTCGTGAAGCTGGATTCGCTAGTAATCGCGCATCAGCCATGGCGCG
GTGAATACGTTCCCGGGCCTTGTACACACCGCCCGTCAAGCCATGAAAGCCGGGGG
TACCTGAAGTACGTAACCGCAAGGAGCGTCCTAGGGTAAAACTGGTAATTGGGGCT
NB2-A13NA Coprococcus catus
(SEQ ID NO: 37)
ATGAGAGTTTGATCCTGGCTCAGGATGAACGCTGGCGGCGTGCTTAACACATGCAA
GTCGAACGGACGATGAAGAGCTTGCTTTTCAGAGTTAGTGGCGGACGGGTGAGTAA
CGCGTGGGTAACCTGCCTCATACAGGGGGATAGCAGCTGGAAACGGCTGATAAAAC
CGCATAAGCGCACAGCATCGCATGATGCAGTGTGAAAAACTCCGGTGGTATGAGAT
GGACCCGCGTCTGATTAGCTGGTTGGTGAGGTAACGGCCCACCAAGGCGACGATCA
GTAGCCGGCCTGAGAGGGTGACCGGCCACATTGGGACTGAGACACGGCCCAAACTC
CTACGGGAGGCAGCAGTGGGGAATATTGCACAATGGGGGAAACCCTGATGCAGCGA
CGCCGCGTGAAGGAAGAAGTATCTCGGTATGTAAACTTCTATCAGCAGGGAAGATA
ATGACGGTACCTGACTAAGAAGCCCCGGCTAACTACGTGCCAGCAGCCGCGGTAAT
ACGTAGGGGGCAAGCGTTATCCGGATTTACTGGGTGTAAAGGGAGCGTAGGCGGCG
GAGCAAGTCAGAAGTGAAAGCCCGGGGCTCAACCCCGGGACGGCTTTTGAAACTGC
CCTGCTTGATTTCAGGAGAGGTAAGCGGAATTCCTAGTGTAGCGGTGAAATGCGTAG
ATATTAGGAGGAACACCAGTGGCGAAGGCGGCTTACTGGACTGACAATGACGCTGA
GGCTCGAAAGCGTGGGGAGCAAACAGGATTAGATACCCTGGTAGTCCACGCCGTAA
ACGATGAATACTAGGTGTCGGGGCTCATAAGAGCTTCGGTGCCGCAGCAAACGCAA
TAAGTATTCCACCTGGGGAGTACGTTCGCAAGAATGAAACTCAAAGGAATTGACGG
GGACCCGCACAAGCGGTGGAGCATGTGGTTTAATTCGAAGCAACGCGAAGAACCTT
ACCAGGCCTTGACATCCCGGTGACCGTCCCGTAATGGGGACCTCTCTTCGGAGCACC
GGTGACAGGTGGTGCATGGTTGTCGTCAGCTCGTGTCGTGAGATGTTGGGTTAAGTC
CCGCAACGAGCGCAACCCCTATGTTCAGTAGCCAGCAGGTAAAGCTGGGCACTCTG
GACAGACTGCCGGGGATAACCCGGAGGAAGGCGGGGATGACGTCAAATCATCATGC
CCCTTACGGCCTGGGCTACACACGTGCTACAATGGCGTAAACAAAGGGAAGCGAGA
GGGTGACCTGGAGCGAATCCCAAAAATAACGTCCCAGTTCGGACTGTAGTCTGCAA
CCCGACTACACGAAGCTGGAATCGCTAGTAATCGCGAATCAGCATGTCGCGGTGAA
TACGTTCCCGGGTCTTGTACACACCGCCCGTCACACCATGGGAGTTGGAAATGCCCG
AAGTCAGTGACCTAACCGCAAGGGAGGAGCTGCCGAAGGTGGAGCCGATGACTGGG
GTGAAGTCGTAACAAGGTAGCCGTATCGGAAGGTGCGGCTGGATCACCTCCTTTCTA
AGGAA
NB2-B16TSAB Bifidobacterium adolescentis
(SEQ ID NO: 38)
TGTGGAGGGTTCGATTCTGGCTCAGGATGAACGCTGGCGGCGTGCTTAACACATGCA
AGTCGAACGGGATCCCAGGAGCTTGCTCCTGGGTGAGAGTGGCGAACGGGTGAGTA
ATGCGTGACCGACCTGCCCCATACACCGGAATAGCTCCTGGAAACGGGTGGTAATG
CCGGATGCTCCAGTTGACCGCATGGTCCTCTGGGAAAGCTTTTGCGGTATGGGATGG
GGTCGCGTCCTATCAGCTTGATGGCGGGGTAACGGCCCACCATGGCTTCGACGGGTA
GCCGGCCTGAGAGGGCGACCGGCCACATTGGGACTGAGATACGGCCCAGACTCCTA
CGGGAGGCAGCAGTGGGGAATATTGCACAATGGGCGCAAGCCTGATGCAGCGACGC
CGCGTGCGGGATGACGGCCTTCGGGTTGTAAACCGCTTTTGACTGGGAGCAAGCCCT
TCGGGGTGAGTGTACCTTTCGAATAAGCACCGGCTAACTACGTGCCAGCAGCCGCG
GTAATACGTAGGGTGCAAGCGTTATCCGGAATTATTGGGCGTAAAGGGCTCGTAGG
CGGTTCGTCGCGTCCGGTGTGAAAGTCCATCGCTTAACGGTGGATCCGCGCCGGGTA
CGGGCGGGCTTGAGTGCGGTAGGGGAGACTGGAATTCCCGGTGTAACGGTGGAATG
TGTAGATATCGGGAAGAACACCAATGGCGAAGGCAGGTCTCTGGGCCGTCACTGAC
GCTGAGGAGCGAAAGCGTGGGGAGCGAACAGGATTAGATACCCTGGTAGTCCACGC
CGTAAACGGTGGATGCTGGATGTGGGGACCATTCCACGGTCTCCGTGTCGGAGCCA
ACGCGTTAAGCATCCCGCCTGGGGAGTACGGCCGCAAGGCTAAAACTCAAAGAAAT
TGACGGGGGCCCGCACAAGCGGCGGAGCATGCGGATTAATTCGATGCAACGCGAAG
AACCTTACCTGGGCTTGACATGTTCCCGACAGCCCCAGAGATGGGGCCTCCCTTCGG
GGCGGGTTCACAGGTGGTGCATGGTCGTCGTCAGCTCGTGTCGTGAGATGTTGGGTT
AAGTCCCGCAACGAGCGCAACCCTCGCCCTGTGTTGCCAGCACGTCGTGGTGGGAA
CTCACGGGGGACCGCCGGGGTCAACTCGGAGGAAGGTGGGGATGACGTCAGATCAT
CATGCCCCTTACGTCCAGGGCTTCACGCATGCTACAATGGCCGGTACAACGGGATGC
GACACCGCGAGGTGGAGCGGATCCCTTAAAACCGGTCTCAGTTCGGATTGGAGTCT
GCAACCCGACTCCATGAAGGCGGAGTCGCTAGTAATCGCGGATCAGCAACGCCGCG
GTGAATGCGTTCCCGGGCCTTGTACACACCGCCCGTCAAGTCATGAAAGTGGGTAGC
ACCCGAAGCCGGTGGCCCAACCTTTTGGGGGGAGCCGTCTAAGGTGAGACTCGTGA
TTGGGACTAAGTCGTAACAAGGTAGCCGTACCGGAAGGTGCGGCTGGATCACCTCC
TTT
NB2-B13DCM Collinsella aerofaciens
(SEQ ID NO: 39)
CGGAGAGTTCGATCCTGGCTCAGGATGAACGCTGGCGGCGCGCCTAACACATGCAA
GTCGAACGGCACCCACCTCCGGGTGGAAGCGAGTGGCGAACGGCTGAGTAACACGT
GGAGAACCTGCCCCCTCCCCCGGGATAGCCGCCCGAAAGGACGGGTAATACCGGAT
ACCCCGGGGTGCCGCATGGCACCCCGGCTAAAGCCCCGACGGGAGGGGATGGCTCC
GCGGCCCATCAGGTAGACGGCGGGGTGACGGCCCACCGTGCCGACAACGGGTAGCC
GGGTTGAGAGACCGACCGGCCAGATTGGGACTGAGACACGGCCCAGACTCCTACGG
GAGGCAGCAGTGGGGAATCTTGCGCAATGGGGGGAACCCTGACGCAGCGACGCCGC
GTGCGGGACGGAGGCCTTCGGGTCGTAAACCGCTTTCAGCAGGGAAGAGTCAAGAC
TGTACCTGCAGAAGAAGCCCCGGCTAACTACGTGCCAGCAGCCGCGGTAATACGTA
GGGGGCGAGCGTTATCCGGATTCATTGGGCGTAAAGCGCGCGTAGGCGGCCCGGCA
GGCCGGGGGTCGAAGCGGGGGGCTCAACCCCCCGAAGCCCCCGGAACCTCCGCGGC
TTGGGTCCGGTAGGGGAGGGTGGAACACCCGGTGTAGCGGTGGAATGCGCAGATAT
CGGGTGGAACACCGGTGGCGAAGGCGGCCCTCTGGGCCGAGACCGACGCTGAGGCG
CGAAAGCTGGGGGAGCGAACAGGATTAGATACCCTGGTAGTCCCAGCCGTAAACGA
TGGACGCTAGGTGTGGGGGGACGATCCCCCCGTGCCGCAGCCAACGCATTAAGCGT
CCCGCCTGGGGAGTACGGCCGCAAGGCTAAAACTCAAAGGAATTGACGGGGGCCCG
CACAAGCAGCGGAGCATGTGGCTTAATTCGAAGCAACGCGAAGAACCTTACCAGGG
CTTGACATATGGGTGAAGCGGGGGAGACCCCGTGGCCGAGAGGAGCCCATACAGGT
GGTGCATGGCTGTCGTCAGCTCGTGTCGTGAGATGTTGGGTTAAGTCCCGCAACGAG
CGCAACCCCCGCCGCGTGTTGCCATCGGGTGATGCCGGGAACCCACGCGGGACCGC
CGCCGTCAAGGCGGAGGAGGGCGGGGACGACGTCAAGTCATCATGCCCCTTATGCC
CTGGGCTGCACACGTGCTACAATGGCCGGTACAGAGGGATGCCACCCCGCGAGGGG
GAGCGGATCCCGGAAAGCCGGCCCCAGTTCGGATTGGGGGCTGCAACCCGCCCCCA
TGAAGTCGGAGTTGCTAGTAATCGCGGATCAGCATGCCGCGGTGAATGCGTTCCCGG
GCCTTGTACACACCGCCCGTCACACCACCCGAGTCGTCTGCACCCGAAGTCGCCGGC
CCAACCGTCAAGGGGGGAGGCGCCGAAGGTGTGGAGGGTGAGGGGGGTGAAGTCG
TAACAAGGTAGCCGTACCGGAAGGTGCGGCTGGATCACCTCCTTT
14LG Acidaminococcus intestini
(SEQ ID NO: 40)
GACTTCACCCCAATCATNGGCCCCANTTAGACAGCTGACTCCTAAAAGGTTATCTCA
CCGGCTTCGGGTGTTACCAACTTTCGTGGTGTGACGGGCGGTGTGTACAAGGCCCGG
GAACGTATTCACCGCAGTATGCTGACCTGCGATTACTAGCGATTCCAACTTCACGTA
GGCGGGTTGCAGCCTACGATCCGAACTGGGGTCGGGTTTCTGGGATTTGCTCCACCT
CGCGGTTTCGCTGCCCTTTGTTGCCGACCATTGTAGTACGTGTGTAGCCCAAGACAT
AAGGGGCATGATGACTTGACGTCATCCCCGCCTTCCTCCAAGTTATCCCTGGCAGTC
TCCTATGAGTCCCCGCCTTTACGCGCTGGTAACATAGGATAGGGGTTGCGCTCGTTG
CGGGACTTAACCCAACATCTCACGACACGAGCTGACGACAGCCATGCACCACCTGT
TTTCGTGTCCCCGAAGGGAGGGACCTATCTCTAGGTCTTTCACTCAATGTCAAGCCTT
GGTAAGGTTCTTCGCGTTGCGTCGAATTAAACCACATACTCCACCGCTTGTGCGGGC
CCCCGTCAATTCCTTTGAGTTTCAATCTTGCGATCGTAGTCCCCAGGCGGGATACTTA
TTGCGTTAACTCCGGCACAGAAGGGGTCGATACCTCCTACACCTAGTATCCATCGTT
TACGGCCAGGACTACCGGGGTATCTAATCCCGTTTGCTACCCTGGCTTTCGCATCTC
AGCGTCAGACACAGTCCAGAAAGGCGCCTTCGCCACTGGTGTTCCTCCCAATATCTA
CGCATTTCACCGCTACACTGGGAATTCCCCTTTCCTCTCCTGCACTCAAGACTTCCAG
TATCCAACGCCATACGGGGTTAAGCCCCGCATTTTCACGTCAGACTTAAAAGCCCGC
CTACATGCTCTTTACGCCCAATAATTCCGGACAACGCTTGCCACCTACGTATTACCG
CGGCTGCTGGCACGTAGTTAGCCGTGGCTTCCTCGTTAGGTACCGTCAACACCATGA
CCTGTTCGAACACGGTGCTTTCGTCCCTAACAACAGAGTTTTACAATCCGAAGACCT
TCATCACTCACGCGGCGTTGCTCCGTCAGACTTTCGTCCATTGCGGAAGATTCCCCA
CTGCTGCCTCCCGTAGGAGTTTGGGCCGTGTCTCAGTCCCAATGTGGCCGTTCATCCT
CTCAGACCGGCTACTGATCATCGCCTTGGTGAGCCGTTACCCCACCAACTAGCTAAT
CAGACGCGGGCCCATCTTCCAGCGATAGCTTGCAAGCAGAGGCCATCTTTCCTCCCT
CCTCCATGCGGAGGAGGGAGCACATTCGGTATTAGCATCCCTTTCGGAATGTTGTCC
CCAACTGGAGGGCAGGTTGCCCACGCGTTACTCACCCGTTCGCCACTAAGAACTTAC
CGAAATAAGTTCTCCGTTCGACTTGCATGTGTTAAGCACGCCGCCAGCGTTCGTCCT
GAGCC

1. An anhydrous composition comprising a co-selected microbiota, wherein the co-selected microbiota comprises a plurality of bacterial species consisting of each of the bacterial species listed in Table 1, and optionally, at least one additional bacterial species,
wherein the bacterial species listed in Table 1 are in powder-form,
wherein the powder-form has a moisture content of less than 5% wt/wt in the anhydrous composition, and
wherein the co-selected microbiota exhibits resistance to perturbational stress.

2-7. (canceled)

8. The anhydrous composition of claim 1, wherein the co-selected microbiota comprises at least 25% Gram-negative bacterial species.

9. The anhydrous composition of claim 1, wherein the co-selected microbiota comprises at least 50% Gram-positive bacterial species.

10. The anhydrous composition of claim 1, wherein the co-selected microbiota comprises at least 65% bacterial species within the Firmicutes phylum.

11. The anhydrous composition of claim 1, wherein the co-selected microbiota comprises at least 5% bacterial species within the Bacteroidetes phylum.

12. The anhydrous composition of claim 1, wherein the co-selected microbiota comprises a sub-group as set forth in any one of Tables 3, 4, or 5 with respect to category and/or functional properties.

13. The anhydrous composition of claim 1, wherein the bacterial species are in a state of suspended animation.

14. The anhydrous composition of claim 1, further comprising a pharmaceutically acceptable carrier.

15. The anhydrous composition of claim 14, wherein the pharmaceutically acceptable carrier is cellulose.

16. The anhydrous composition of claim 1, wherein the anhydrous composition is encapsulated in a capsule.

17. The anhydrous composition of claim 16, wherein the anhydrous composition is encapsulated in a double capsule.

18. The anhydrous composition of claim 1, wherein the at least one additional bacterial species is a species in the Acidaminococcus genus.

19. The anhydrous composition of claim 18, wherein the species in the Acidaminococcus genus is Acidaminococcus intestini or Acidaminococcus fermentans.

20. The anhydrous composition of claim 1, further comprising a prebiotic.

21. A method for treating a mammalian subject afflicted with a disease or disorder associated with dysbiosis, the method comprising: administering a therapeutically effective amount of an anhydrous composition of claim 1 to the mammalian subject, wherein the therapeutically effective amount improves relative ratios of microorganisms in the mammalian subject, thereby treating the mammalian subject.

22. The method of claim 21, wherein the disease or disorder associated with dysbiosis is Clostridium difficile (Clostridioides difficile) infection, Crohn's disease, irritable bowel syndrome (IBS) or spastic colon, idiopathic ulcerative colitis, mucous colitis, collagenous colitis, inflammatory bowel disease in general, microscopic colitis, antibiotic-associated colitis, idiopathic or simple constipation, diverticular disease, or AIDS enteropathy.

23-62. (canceled)


SEQUENCE LISTING (82 sequences)

SEQ ID NO: 1 - DNA, 1528 bp, Artificial Sequence
  Description of Artificial Sequence Synthetic polynucleotide
acgaagagtttgatcctggctcaggatgaacgctagcgacaggcttaacacatgcaagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactccttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtagccgtaccggaaggtgcggctggaacacctccttt

SEQ ID NO: 2 - DNA, 1424 bp, Artificial Sequence
  Description of Artificial Sequence Synthetic polynucleotide
ctgagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgctcggctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggcttcggtgccgccgctaacgcagtaagtattccacctggggagtacgttcgcaagaatgaaactcaaaggaattgacggggacccgcacaagcggtggagcatgtggtttaattcgaagcaacgcgaagaaccttaccaggtcttgacatccttctgaccgcaccttaatcggtgctttccttcgggacagaagagacaggtggtgcatggttgtcgtcagctcgtgtcgtgagatgttgggttaagtcccgcaacgagcgcaacccctatcttcagtagccagcaggtaaggctgggcactctggagagactgccagggataacctggaggaaggtggggacgacgtcaaatcatcatgccccttatgatctgggcgacacacgtgctacaatggcggtcacagagtgaggcgaacccgcgagggggagcaaaccacaaaaaggccgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagccagtgacccaaccttttggagggagctgtcgaaggtggagccggtaactggggtgaagtcgtaacaaggtagccgtatcggaaggtgcggctggatcacctccttt

SEQ ID NO: 3 - DNA, 1301 bp, Artificial Sequence
  Description of Artificial Sequence Synthetic polynucleotide
ctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatgggcgtaagcctgaaccagccaagtcgcgtgagggatgaaggttctatggatcgtaaacctcttttataagggaataaagtgcgggacgtgtcctgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcttagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacagtgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtgatgctgaggactctggtgggactgccagcgtaagctgcgaggaaggcggggatgacgtcaaatcagcacggcccttacatccggggcgacacacgtgttacaatggcgtggacaaagggatgccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgta

SEQ ID NO: 4 - DNA, 1656 bp, Artificial Sequence
  Description of Artificial Sequence Synthetic polynucleotide
attggagagtttgatcctggctcaggacgaacgctggcggcatgcctaacacatgcaagtcgaacggagaaagttcaacaccaagtatttcatccgctgaagtgtagcggtaaaaattgcgaagcaatttttactacgcattaaaagcatgaactaacacggtggttgaagtattaggtgttgaactttcttagtggcgaacgggtgagtaacgcgtgggcaacctgccctctagatggggacaacatcccgaaaggggtgctaataccgaatgtgacagcaatctcgcatgaggatgctgtgaaagatggcctctatttataagctatcgctagaggatgggcctgcgtctgattagctagttggtggggtaacggcctaccaaggcgatgatcagtagccggtctgagaggatgaacggccacattgggactgagacacggcccagactcctacgggaggcagcagtggggaatcttccgcaatgggcgaaagcctgacggagcaatgccgcgtgagtgatgaaggaattcgttccgtaaagctcttttgtttatgacgaatgtgcagattgtaaataatgatctgtaatgacggtagtaaacgaataagccacggctaactacgtgccagcagccgcggtaatacgtaggtggcgagcgttgtccggaattattgggcgtaaagagcatgtaggcggttttttaagtctggagtgaaaatgcggggctcaaccccgtatggctctggatactggaagacttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacgtccgcaaggatgaaactcaaaggaattgacgggggcccgcacaagcggtggagtatgtggtttaattcgacgcaacgcgaagaaccttaccaaggcttgacattgaatgaccgctccagagatggagctttcccttcggggacatgaaaacaggtggtgcatggctgtcgtcagctcgtgtcgtgagatgttgggttaagtcccgcaacgagcgcaacccctatcctatgttaccagcgggtaatgccggggactcataggagactgccaaggacaacttggaggaaggcggggatgacgtcaagtcatcatgccccttatgtcttgggctacacacgtactacaatggtcggcaacagagggaagcaaagccgtgaggcagagcaaaccccagaaacccgatcccagttcggattgcaggctgcaactcgcctgcatgaagtcggaatcgctagtaatcgcaggtcagcatactgcggtgaatacgttcccgggccttgtacacaccgcccgtcacaccacgaaagttggtaacacccgaagccggtggggtaaccgtaaggagccagccgtctaaggtggggccgatgattggggtgaagtcgtaacaaggtagccgtatcggaaggtgcggctggatcacctccttt

SEQ ID NO: 5 - DNA, 1432 bp, Artificial Sequence
  Description of Artificial Sequence Synthetic polynucleotide
gagagtttgatcctggctcaggatgaacgctggcggcgtgcttaacacatgcaagtcgagcgaagcacttaggaaagattcttcggatgatttcctatttgactgagcggcggacgggtgagtaacgcgtgggtaacctgcctcatacagggggataacagttagaaatgactgctaataccgcataagaccacagcaccgcatggtgcaggggtaaaaactccggtggtatgagatggacccgcgtctgattagttagttggtggggtaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggagcagcaagtctgatgtgaaaacccggggctcaaccccgggactgcattggaaactgttgatctggagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgactactaggtgtcgggtggcaaagccattcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgacggcagagtaatgtctgcttttctttggaacaccggtgacaggtggtgcatggttgtcgtcagctcgtgtcgtgagatgttgggttaagtcccgcaacgagcgcaacccctatcttcagtagccagcggtaaggccgggcactctggagagactgccagggataacctggaggaaggtggggatgacgtcaaatcatcatgccccttatgagcagggctacacacgtgctacaatggcgtaaacaaagggaagcgaacccgcgagggtgggcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaacc

SEQ ID NO: 6 - DNA, 1538 bp, Artificial Sequence
  Description of Artificial Sequence Synthetic polynucleotide
tttagagagtttgatcctggctcaggacgaacgctggcggcgtgcctaacacatgcaagtcgaacggagatagacgctgaaagggagacagcttgctgtaagaatttcttgtttatcttagtggcggacgggtgagtaacgcgtgagtaacctgcctttcagagggggataacgtctggaaacggacgctaataccgcatgagaccacagcttcacatggagcggcggtcaaaggagcaatccgctgaaagatggactcgcgtccgattagatagttggcggggtaacggcccaccaagtcgacgatcggtagccggactgagaggttgaacggccacattgggactgagacacggcccagactcctacgggaggcagcagtgagggatattggtcaatgggggaaaccctgaaccagcaacgccgcgtgagggaagacggttttcggattgtaaacctctgtcctctgtgaagatagtgacggtagcagaggaggaagctccggctaactacgtgccagcagccgcggtaatacgtagggagcgagcgttgtccggatttactgggtgtaaagggtgcgtaggcggctctgcaagtcagaagtgaaatccatgggcttaacccatgaactgcttttgaaactgtagagcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggtctgaccccctccgtgccggagttaacacaataagtaatccacctggggagtacgaccgcaaggttgaaactcaaaggaattgacgggggcccgcacaagcagtggagtatgtggattaattcgaagcaacgcgaagaaccttaccaggtcttgacatccaactaacgaagcagagatgcattaggtgcccttcggggaaagttgagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggacgacgtcaaatcatcatgccccttatgacctgggcctcacacgtactacaatggccattaacagagggaagcaagcccgcgaggtggagcaaaaccctaaaaatggtctcagttcggatcgtaggctgaaacccgcctgcgtgaagttggaattgctagtaatcgcggatcagcatgccgcggtgaatacgttcccgggccttgtacacaccgcccgtcacaccatgggagccggtaatacccgaagtcagtagtctaaccgcaagggggacgctgccgaaggtaggattggcgactggggtgaagtcgtaacaaggtagccgtatcggaaggtgcggctggatcacctccttt

SEQ ID NO: 7 - DNA, 1522 bp, Artificial Sequence
  Description of Artificial Sequence Synthetic polynucleotide
atggagagtttgatcctggctcaggatgaacgctggcggcgtgcctaatacatgcaagtcgaacgcttcacttcggtgaagagtggcgaacgggtgagtaatacataagtaacctggcatctacagggggataactgatggaaacgtcagctaagaccgcataggtgtagagatcgcatgaactctatatgaaaagtgctacgggactggtagatgatggacttatggcgcattagctggttggtagggtaacggcctaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaagtaattcgttatgtaaacttctgtcatagaggaagaacggtggatatagggaatgatatccaagtgacggtactctataagaaagccacggctaactacgtgccagcagccgcggtaatacgtaggtggcgagcgttatccggaattattgggcgtaaagagggagcaggcggcactaagggtctgtggtgaaagatcgaagcttaacttcggtaagccatggaaaccgtagagctagagtgtgtgagaggatcgtggaattccatgtgtagcggtgaaatgcgtagatatatggaggaacaccagtggcgaaggcgacgatctggcgcataactgacgctcagtcccgaaagcgtggggagcaaataggattagataccctagtagtccacgccgtaaacgatgagtactaagtgttgggagtcaaatctcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatcgatctaaaggctccagagatggagagatagctatagagaagacaggtggtgcatggttgtcgtcagctcgtgtcgtgagatgttgggttaagtcccgcaacgagcgcaacccctgttgccagttgccagcattaagttggggactctggcgagactgccggtgacaagccggaggaaggcggggatgacgtcaaatcatcatgccccttatgacctgggctacacacgtgctacaatggacagagcagagggaagcgaagccgcgaggtggagcgaaacccataaaactgttctcagttcggactgcagtctgcaactcgactgcacgaagatggaatcgctagtaatcgcgaatcagcatgtcgcggtgaatacgttctcgggccttgtacacaccgcccgtcacaccatgagagtcggtaacacccgaagccggtggcctaaccgcaaggaaggagctgtctaaggtgggactgatgattggggtgaagtcgtaacaaggtatccctacgggaacgtggggatggatcacctccttt

SEQ ID NO: 8 - DNA, 1426 bp, Artificial Sequence
  Description of Artificial Sequence Synthetic polynucleotide
actgagtggcggacgggtgagtaacgcgtgggtaacctgcctcatacagggggataacagttggaaacgactgctaataccgcataagcgcacagggtcgcatgacctggtgtgaaaaactccggtggtatgagatggacccgcgtctgattagccagttggtggggtaacggcctaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggtacggcaagtctgatgtgaaagcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccgatgacagaacatgtaatgtgttttctcttcggagcatcggtgacaggtggtgcatggttgtcgtcagctcgtgtcgtgagatgttgggttaagtcccgcaacgagcgcaacccctattcttagtagccagcgggtaagccgggcactctagggagactgccagggataacctggaggaaggtggggatgacgtcaaatcatcatgccccttatgacttgggctacacacgtgctacaatggcgtaaacaaagggaagcgagcctgcgagggggagcaaatctcaaaaataacgtctcagttcggactgcagtctgcaactcgactgcacgaagctggaatcgctagtaatcgcgaatcagaatgtcgcggtgaatacgttcccgggtcttgtacacaccgcccgtcacaccatgggagttggtaatgcccgaagtcagtgacccaaccgcaaggagggagctgccgaaggcaggatcgataactggggtgaagtcgtaacaaggtagccgtatcggaaggtgcggctggatcacctccttt

SEQ ID NO: 9 - DNA, 1509 bp, Artificial Sequence
  Description of Artificial Sequence Synthetic polynucleotide
atggagagtttgattctggctcagaacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagaatcgcaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaagtcgtaacaaggtagccgtaggggaacctgcggctggatcacctccttt

SEQ ID NO: 10 - DNA, 1379 bp, Artificial Sequence
  Description of Artificial Sequence Synthetic polynucleotide
  modified_base (66)..(66): a, c, t, g, unknown or other
  modified_base (72)..(72): a, c, t, g, unknown or other
  modified_base (202)..(202): a, c, t, g, unknown or other
  modified_base (210)..(210): a, c, t, g, unknown or other
  modified_base (218)..(218): a, c, t, g, unknown or other
  modified_base (228)..(229): a, c, t, g, unknown or other
  modified_base (243)..(243): a, c, t, g, unknown or other
  modified_base (246)..(246): a, c, t, g, unknown or other
  modified_base (264)..(264): a, c, t, g, unknown or other
  modified_base (267)..(267): a, c, t, g, unknown or other
  modified_base (285)..(285): a, c, t, g, unknown or other
  modified_base (319)..(319): a, c, t, g, unknown or other
  modified_base (337)..(337): a, c, t, g, unknown or other
  modified_base (346)..(346): a, c, t, g, unknown or other
  modified_base (356)..(356): a, c, t, g, unknown or other
  modified_base (367)..(367): a, c, t, g, unknown or other
  modified_base (372)..(372): a, c, t, g, unknown or other
  modified_base (375)..(375): a, c, t, g, unknown or other
  modified_base (380)..(380): a, c, t, g, unknown or other
  modified_base (395)..(395): a, c, t, g, unknown or other
  modified_base (403)..(403): a, c, t, g, unknown or other
  modified_base (443)..(443): a, c, t, g, unknown or other
  modified_base (481)..(481): a, c, t, g, unknown or other
  modified_base (577)..(577): a, c, t, g, unknown or other
  modified_base (654)..(654): a, c, t, g, unknown or other
  modified_base (671)..(671): a, c, t, g, unknown or other
  modified_base (673)..(673): a, c, t, g, unknown or other
  modified_base (680)..(680): a, c, t, g, unknown or other
  modified_base (684)..(684): a, c, t, g, unknown or other
  modified_base (698)..(698): a, c, t, g, unknown or other
  modified_base (712)..(712): a, c, t, g, unknown or other
  modified_base (755)..(755): a, c, t, g, unknown or other
  modified_base (1052)..(1052): a, c, t, g, unknown or other
  modified_base (1056)..(1056): a, c, t, g, unknown or other
  modified_base (1086)..(1086): a, c, t, g, unknown or other
  modified_base (1138)..(1138): a, c, t, g, unknown or other
  modified_base (1161)..(1162): a, c, t, g, unknown or other
  modified_base (1193)..(1193): a, c, t, g, unknown or other
  modified_base (1199)..(1199): a, c, t, g, unknown or other
  modified_base (1201)..(1201): a, c, t, g, unknown or other
  modified_base (1206)..(1206): a, c, t, g, unknown or other
  modified_base (1208)..(1209): a, c, t, g, unknown or other
  modified_base (1214)..(1214): a, c, t, g, unknown or other
  modified_base (1227)..(1229): a, c, t, g, unknown or other
  modified_base (1234)..(1234): a, c, t, g, unknown or other
  modified_base (1238)..(1238): a, c, t, g, unknown or other
  modified_base (1246)..(1246): a, c, t, g, unknown or other
  modified_base (1263)..(1263): a, c, t, g, unknown or other
  modified_base (1271)..(1271): a, c, t, g, unknown or other
  modified_base (1278)..(1278): a, c, t, g, unknown or other
  modified_base (1289)..(1289): a, c, t, g, unknown or other
  modified_base (1292)..(1292): a, c, t, g, unknown or other
  modified_base (1300)..(1301): a, c, t, g, unknown or other
  modified_base (1303)..(1303): a, c, t, g, unknown or other
  modified_base (1306)..(1306): a, c, t, g, unknown or other
gcttaacacatgcaagtcgaacgagaaggcgtagcaatacgcttgtaaagtggcgaacgggtgagnaacacntgggtaacctaccctcgagtgggggataacccgccgaaaggcgggctaataccgcgtacgcttccgatcttgcgagatcggaaggaaagctgtcccaaggggatggcgctcaaggatgggctcacgtccnatcagctngttggtgnggtaacggcnnaccaaggcgacganggntagctggtctgagaggangancagccacactgggactgngacacggcccagactcctacgggaggcagcagtngggaatcttgcgcaatgngcgaaagcntgacgcagcnacgccgcgtgnggganganggccntcgggttgtaaaccnctttcagnagggacgaatctgacggtacctgcagaagaagccccggcnaactacgtgccagcagccgcggtaatacgtagggggcnagcgttgtccggatttattgggcgtaaagagctcgtaggcggcttggcaagtcgggtgtgaaacctccaggcttaacctggagacgccactcgatnctgccatggctagagtccggtaggggaccacggaattcctggtgtagcggtgaaatgcgcagatatcaggaggaacnccggtggcgaaggcggngntctgggncggnactgacgctgaggngcgaaagcgtgggnagcaaacaggattagataccctggtagtccacgccgtaaacgntgggcactaggtgtgggaccttatcaacgggttccgtgccgtagctaacgcattaagtgccccgcctggggagtacggccgcaaggctaaaactcaaaggaattgacgggggcccgcacaagcggcggagcatgttgcttaattcgatgcaacgcgaagaaccttacctgggttgaactacgcgggaaaagccacagagatgtggtgtccgaaagggcccgcgataggtggtgcatggctgtcgtcagctcgtgtcgtgagatgttgggttaagtcccgcaacgagcgcaacccttntccnatgttgccagcggatcatgccggggactcntgggagactgccggggtcaactcggaggaaggtggggatgacgtcaagtcancatgccccttatgtccagggctnnaaacatgctacaatggccggtacaaagggtngcgagncngcgangnngagcnaatcccataaagnnngtctnagtncggatcgnagtctgcaactcgactncgtgaagncggagtngctagtaatcncgnatcagcanngncgnggtgaatacgttcccgggccttgtacacaccgcccgtcacaccacgaaagttggtaacacccgaagccggtgg

SEQ ID NO: 11 - DNA, 1308 bp, Artificial Sequence
  Description of Artificial Sequence Synthetic polynucleotide
  modified_base (789)..(789): a, c, t, g, unknown or other
gagtaattcggtataggatgggcccgcatctgattagctagttggtgagataacagcccaccaaggcgacgatcagtagccgacctgagagggtgatcggccacattgggactgagacacggcccaaactcctacgggaggcagcagtggggaatattgcacaatgggggaaaccctgatgcagcaacgccgcgtgaaggaagaaggttttcggatcgtaaacttctatcaacagggacgaagaaagtgacggtacctgaataagaagccccggctaactacgtgccagcagccgcggtaatacgtagggggcaagcgttatccggaattactgggtgtaaagggagcgtaggcggcacgccaagccagatgtgaaagcccgaggcttaacctcgcggattgcatttggaactggcgagctagagtacaggagaggaaagcggaattcctagtgtagcggtgaaatgcgtagatattaggaagaacaccagtggcgaaggcggctttctggactgaaactgacgctgaggctcgaaagcgtggggagcaaacaggattagataccctggtagtccacgccgtaaacgatgagtgctaggtgtcggggaggaatcctcggtgccgcagctaacgcaataagcactccacctggggagtacgaccgcaaggttgaaactcaaaggaattgacgggggcccgcacaagcggtggagcatgtggtttaattcgaagcaacgcgaagaaccttaccaaggcttgacatcccgatgaccgctctagagatagagnttctcttcggagcatcggtgacaggtggtgcatggttgtcgtcagctcgtgtcgtgagatgttgggttaagtcccgcaacgagcgcaacccttatccttagtagccatcattgagttgggcactctagggagactgccgtggataacacggaggaaggtggggatgacgtcaaatcatcatgccccttatgtcttgggctacacacgtgctacaatggctggtaacagagtgaagcgagacggcgacgttaagcaaatcacaaaaacccagtcccagttcggattgtagtctgcaactcgactacatgaagctggaatcgctagtaatcgcgaatcagaatgtcgcggtgaatacgttcccgggccttgtacacaccgcccgtcacaccatgggagttggaagcacccgaagtcggtgacctaaccgtaaggaaggagccgccgaaggtgaagccagtgactggggtgaagtcgtaacaaggtagccgtatcggaaggtgcggctggatcacctccttt

SEQ ID NO: 12 - DNA, 1514 bp, Artificial Sequence
  Description of Artificial Sequence Synthetic polynucleotide
atatgagagtttgatcctggctcaggatgaacgctggcggcgtgcctaacacatgcaagtcgagcgagaagctgatgattgacacttcggttgagagaatcagtggaaagcggcggacgggtgagtaacgcgtaggcaacctgccctttgcagagggatagcctcgggaaaccgggattaaaacctcatgatgctgtatgtccgcatgggcagacggtcaaagatttatcggcagaggatgggcctgcgtctgattagttagttggtggggtaacggcctaccaaggcaacgatcagtagccgacctgagagggtgatcggccacattggaactgagacacggtccaaactcctacgggaggcagcagtggggaatattgcacaatgggggaaaccctgatgcagcaacgccgcgtgagcgaagaaggcctttgggtcgtaaagctctgtccttggggaagaaaaaatgacggtacccaaggaggaagccccggctaactacgtgccagcagccgcggtaatacgtagggggcaagcgttatccggaattattgggcgtaaagagtatgtaggtggtttcttaagcgcagggtataaggcaatggcttaaccattgttcgccccgtgaactgagagacttgagtgctggagaggaaagcggaattcctagtgtagcggtgaaatgcgtagatattaggaggaacaccagtggcgaaggcggctttctggacagtaactgacactgagatacgaaagcgtggggagcaaacaggattagataccctggtagtccacgccgtaaacgatgagcactaggtgtcgggctcgcaagagttcggtgccggagttaacgcattaagtgctccgcctggggagtacgcacgcaagtgtaaaactcaaaggaattgacggggacccgcacaagcagcggagcatgtggtttaattcgaagcaacgcgaagaaccttaccagggcttgacatccctccgaccggtccttaatcggacctttctacggacgggggagacaggtggtgcatggttgtcgtcagctcgtgtcgtgagatgttgggttaagtcccgcaacgagcgcaacccttgtcattagttgctaacagtaagatgagaactctaatgagactgccgtggataacacggaggaaggtggggatgacgtcaaatcatcatgccccttatgtcctgggctacacacgtgctacaatggtcggtacaaagagaagcaagaccgcgaggtggagcaaatctcaaaaaccgatcccagttcggattgcaggctgcaactcgcctgcatgaagtcggagttgctagtaatcgcagatcagaatgctgcggtgaatgcgttcccgggtcttgtacacaccgcccgtcacaccatggaagttgggggcgcccgaagtcggctagtaaataggctgcctaaggcgaaatcaatgactggggtgaagtcgtaacaaggtagccgtatcggaaggtgcggctggatcacctccttt

SEQ ID NO: 13 - DNA, 1460 bp, Artificial Sequence
  Description of Artificial Sequence Synthetic polynucleotide
aacgagagtttgatcctggctcaggatgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgactactaggtgtcgggtggcaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccgctgaccggacggtaatgcgtccttcccttcggggcagcggagacaggtggtgcatggttgtcgtcagctcgtgtcgtgagatgttgggttaagtcccgcaacgagcgcaacccctatctttagtagccagcggccaggccgggcactctagagagactgccggggataacccggaggaaggtggggatgacgtcaaatcatcatgccccttatgagcagggctacacacgtgctacaatggcgtaaacaaagggaagcgagaccgcgaggtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgg

SEQ ID NO: 14 - DNA, 1432 bp, Artificial Sequence
  Description of Artificial Sequence Synthetic polynucleotide
attttgtgactgagtggcggacgggtgagtaacgcgtgggtaacctgccttgtacagggggataacagttggaaacggctgctaataccgcataagcgcacagcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggttcggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggagctgtcgaaggcaggctcgataactggggtgaagtcgtaacaaggtagccgtatcggaaggtgcggctggatcacctccttt

SEQ ID NO: 15 - DNA, 1524 bp, Artificial Sequence
  Description of Artificial Sequence Synthetic polynucleotide
atgaagagtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtatttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggtaccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatccctaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtagccgtaccggaaggtgcggctggaacacctccttt

SEQ ID NO: 16 - DNA, 1426 bp, Artificial Sequence
  Description of Artificial Sequence Synthetic polynucleotide
actgagtggcggacgggtgagtaacgcgtgggtaacctgcctcacacagggggataacagttggaaacggctgctaataccgcataagcgcacagtaccgcatggtacagtgtgaaaaactccggtggtgtgagatggacccgcgtctgattagctagttggcagggcaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggaaggctaagtctgatgtgaaagcccggggctcaaccccggtactgcattggaaactggtcatctagagtgtcggaggggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcggaaagcacagcttttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatcccggtgaccggacagtaatgtgtccttttcttcggaacaccggtgacaggtggtgcatggttgtcgtcagctcgtgtcgtgagatgttgggttaagtcccgcaacgagcgcaacccttatccccagtagccagcattttggatgggcactctgaggagactgccagggataacctggaggaaggtggggatgacgtcaaatcatcatgccccttatgacttgggctacacacgtgctacaatggcgtaaacaaagggaagcgagaccgtgaggtggagcaaatcccaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttggaaatgcccgaagtcagtgacccaaccgcaaggagggagctgccgaaggcaggttcgataactggggtgaagtcgtaacaaggtagccgtatcggaaggtgcggctggatcacctccttt

SEQ ID NO: 17 - DNA, 1532 bp, Artificial Sequence
  Description of Artificial Sequence Synthetic polynucleotide
acgagagtttgatcctggctcaggacgaacgctggcggcgtgcctaacacatgcaagtcgaacgagaatctttgggatgattctttcgggatgaattccaaagaggaaagtggcggacgggcgagtaacgcgtgagtaacctgcccataagagggggataatccatggaaacgtggactaataccgcatattgtagttaagttgcatgacttgattatgaaagatttatcgcttatggatggactcgcgtcagattagatagttggtgaggtaacggctcaccaagtcaacgatctgtagccgaactgagaggttgatcggccgcattgggactgagacacggcccagactcctacgggaggcagcagtggggaatattgcgcaatgggggcaaccctgacgcagcaacgccgcgtgcaggaagaaggtcttcggattgtaaactgttgtcgcaagggaagaagacagtgacggtaccttgtgagaaagtcacggctaactacgtgccagcagccgcggtaatacgtaggtgacaagcgttgtccggatttactgggtgtaaagggcgcgtaggcggactgtcaagtcagtcgtgaaataccggggcttaaccccggggctgcgattgaaactgacagccttgagtatcggagaggaaagcggaattcctagtgtagcggtgaaatgcgtagatattaggaggaacaccagtggcgaaggcggctttctggacgacaactgacgctgaggcgcgaaagtgtggggagcaaacaggattagataccctggtagtccacaccgtaaacgatggatactaggtgtaggaggtatcgaccccttctgtgccgcagttaacacaataagtatcccacctggggagtacgaccgcaaggttgaaactcaaaggaattgacgggggcccgcacaagcagtggagtatgtggtttaattcgaagcaacgcgaagaaccttacctgggcttgacatccctggaatcgagtagagatacttgagtgccttcgggaatcaggtgacaggtggtgcatggttgtcgtcagctcgtgtcgtgagatgttgggttaagtcccgcaacgagcgcaacccctattgtcagttgccatcattaagttgggcactctggcgagactgccggtgacaaatcggaggaaggtggggacgacgtcaaatcatcatgccccttatgcccagggctacacacgtactacaatggccgataacaaagtgcagcgaaaccgtgaggtggagcgaatcacaaaactcggtctcagttcagattgcaggctgcaactcgcctgcatgaagttggaattgctagtaatcgcggatcagaatgccgcggtgaatacgttcccgggccttgtacacaccgcccgtcacaccatgagagtcgataacacccgaagcctgtgagctaaccttttaggaggcagcagtcgaaggtggggttgatgattggggtgaagtcgtaacaaggtagccgtatcggaaggtgcggctggatcacctccttt

SEQ ID NO: 18 - DNA, 1437 bp, Artificial Sequence
  Description of Artificial Sequence Synthetic polynucleotide
cggcgaccggcgcacgggtgagtaacacgtatgcaatccacctgtaacagggggataacccggagaaatccggactaataccccataatatgggcgctccgcatggagagcccattaaagagagcaatcttggttacagacgagcatgcgctccattagccagttggcggggtaacggcccaccaaggcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtacgctacgtgtagcgtattgcaagtatccgaagaaaaagcatcggctaactccgtgccagcagccgcggtaatacggaggatgcaagcgttatccggatttattgggtttaaagggtgcgtaggcggcacgccaagtcagcggtgaaatttccgggctcaacccggagtgtgccgttgaaactggcgagctagagtgcacaagaggcaggcggaatgcgtggtgtagcggtgaaatgcatagatatcacgcagaaccccgattgcgaaggcagcctgctagggtgaaacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacgggggcccgcacaagcggaggaacatgtggtttaattcgatgatacgcgaggaaccttacccgggctcaaacgcagggggaatatatatgaaagtatatagctagcaatagtcacctgcgaggtgctgcatggttgtcgtcagctcgtgccgtgaggtgtcggcttaagtgccataacgagcgcaacccctatggacagttactaacgggtgaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgaatcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacgaaggtaataccggtaactggggctaagtcgtaacaaggtagccgtaccggaaggtgcggctggaacacctccttt

SEQ ID NO: 19 - DNA, 1539 bp, Artificial Sequence
  Description of Artificial Sequence Synthetic polynucleotide
ttccttagaaaggaggtgatccagccgcaccttccgatacggctaccttgttacgacttcaccccagttatcagtcccgccttcggcagctccctccttrcggttgggtcactgacttcgggcgttaccaactcccatggtgtgacgggcggtgtgtacaagacccgggaacgtattcaccgcgacattctgattcgcgattactagcgattccagcttcatgtagtcgagttgcagactacaatccgaactgagacgttatttttgagatttgcttaagctcacactctcgcttccctttgtttacgccattgtagcacgtgtgtagcccaagtcataaggggcatgatgatttgacgtcatccccaccttcctccaggttatccctggcagtctctccagagtgcccgaccgaatcgctggctactgaagataagggttgcgctcgttgcgggacttaacccaacatctcacgacacgagctgacgacaaccatgcaccacctgtcaccgatgctccgaagagaaggyyccattacrraccggtcatcgggatgtcaagacttggtaaggttcttcgcgttgcttcgaattaaaccacatgctccaccgcttgtgcgggtccccgtcaattcctttgagtttcattcttgcgaacgtactccccaggtggaatacttattgcgtttgcggcggcaccgaagagctgtgctccccgacacctagtattcatcgtttacggcgtggactaccagggtatctaatcctgtttgctccccacgctttcgagcctcaacgtcagttactgtccagtaagccgccttcgccactggtgttcctcctaatatctacgcatttcaccgctacactaggaattccgcttacctctccagcactctagccaaacagtttcaaaagcagtcccggggttgagccccagcctttcacttctgacttgcttarccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcactcacgcggcgtcgctgcatcagggtttcccccattgtgcaatattccccactgctgcctcccgtaggagtttgggccgtgtctcagtcccaatgtggccgttcaccctctcaggccggctatggatcgtcgccttggtaggccgttaccctgccaactagctaatccaacgcgggtccatctcacaccgataaatcttttccgtccgggccatgcggccctagcgggttatgcggtattagcggtcgtttccaactgttatccccctgtgtgaggcaggttacccacgcgttactcacccgtccgccactaagtcgcaagagaaatcatccgaagaatcaatctcaagcgcttcgttcgacttgcatgtgttaagcacgccgccagcgttcatcctgagccaggatcaaactctcgattaa

SEQ ID NO: 20 - DNA, 1439 bp, Artificial Sequence
  Description of Artificial Sequence Synthetic polynucleotide
  modified_base (1370)..(1370): a, c, t, g, unknown or other
atgaagagtttgatcctggctcaggatgaacgctagctacaggcttaacacatgcaagtcgaggggcagcatgacctagcaataggttgatggcgaccggcgcacgggtgagtaacacgtatccaacctaccggttattccgggatagcctttcgaaagaaagattaataccggatagtataacgagaaggcatcttcttgttattaaagaatttcgataaccgatggggatgcgttccattagtttgttggcggggtaacggcccaccaagacatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtgagccacgtgtggctttttgtatgtaccatacgaataaggatcggctaactccgtgccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaatatagtggaaacattatagccgcaaggcatttgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacagcgatgtgatgctaatcccaaaagcctctctcagttcggattggagtctgcaacccgactccatgaagctggattcgctagtaatcgcgcatcagccacggcgcggtgaatacgttcncgggccttgtacacaccgcccgtcaagccatgaaagccgggggtacctgaagtccgtaaccgcaaggag

SEQ ID NO: 21 - DNA, 1438 bp, Artificial Sequence
  Description of Artificial Sequence Synthetic polynucleotide
tattgagagtttgatcctggctcaggatgaacgctggcggcgtgcttaacacatgcaagtcgaacggggtgctcatgacggaggattcgtccaacggattgagttacctagtggcggacgggtgagtaacgcgtgaggaacctgccttggagagggggataacactccgaaaggagtgctaataccgcatgatgcagttgggtcgcatggctctgactgccaaagatttatcgctctgagatggcctcgcgtctgattagctagtaggtggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcagggacgaaacaaatgacggtacctgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaaaaccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccg

SEQ ID NO: 22 - DNA, 1326 bp, Artificial Sequence
  Description of Artificial Sequence Synthetic polynucleotide
  modified_base (462)..(462): a, c, t, g, unknown or other
  modified_base (806)..(806): a, c, t, g, unknown or other
  modified_base (999)..(999): a, c, t, g, unknown or other
gcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattnctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagnttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatnaccagggctacacacgtgctacaatggcgtaaacaaagagacgcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctccttt

SEQ ID NO: 23 - DNA, 1550 bp, Artificial Sequence
  Description of Artificial Sequence Synthetic polynucleotide
ttcgcttccctctgtttacgccattgtagcacgtgtgtagcccaaatcataaggggcatgatgatttgacgtcatccccaccttcctccaggttatccctggcagtctcctcagagtgcccaccattacatgctggctactggggataggggttgcgctcgttgcgggacttaacccaacatctcacgacacgagctgacgacaaccatgcaccacctgtcttgcctgtcccgaaggaaaggtgacgttactcaccggtcaggcagatgtcaagacttggtaaggttcttcgcgttgcttcgaattaaaccacatgctccaccgcttgtgcgggtccccgtcaattcctttgagtttcattcttgcgaacgtactccccaggtggaatacttaatgcgtttgcggcggcaccgaagagctgtgctccccgacacctagtattcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacccctccggcactcaagcttaacagtttccaatgcagtcccggggttaagccccagcctttcacatcagacttgttatgccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattccccactgctgcctcccgtaggagtctgggccgtgtctcagtcccaatgtggccgttcaccctctcaggccggctatggatcgtcgctttggtaggccgttaccctgccaactggctaatccaacgcgggtccatcttataccacctcagtttttcacaccgggccatgcggccctgtgcgcttatgcggtattagcagccatttctgactgttatccccctgtataaggcaggttacccacgcgttactcacccgtccgccactaggattaaatcaaatctgccgaagcttcaataaaataatccccgttcgacttgcatgtgttaagcacgccgccagcgttcatcctgagccaggatcaaactctctgataaagtttgatgtctcaagacaaccaactagcttagttatctctcgtcattactgttttaaagttcattcttccgaatgtgattgtaaaagaattttcgagaatcgtatgtgtttcactgtttagttatcaatgttcattgctttttactgtctctcgacagcttatttactttaccacatctttttttgtttgtcaacaacttttttgaagtttttcaaactttttttccgaagatgaagttttgtcatccgtgttgacgacttgactactttatcatagataagtcagtttgtcaacagg

SEQ ID NO: 24 - DNA, 1523 bp, Artificial Sequence
  Description of Artificial Sequence Synthetic polynucleotide
gtggagggttcgattctggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctccttt

SEQ ID NO: 25 - DNA, 1525 bp, Artificial Sequence
  Description of Artificial Sequence Synthetic polynucleotide
gagagtttgatcctggctcaggatgaacgctggcggcgtgcttaacacatgcaagtcgaacgaagcacctggatttgattcttcggatgaagatccttgtgactgagtggcggacgggtgagtaacgcgtgggtaacctgcctcatacagggggataacagttagaaatgactgctaataccgcataagaccacagggtcgcatgacctggtgggaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcatgtaatgatgccttttcttcggaacaccggtgacaggtggtgcatggttgtcgtcagctcgtgtcgtgagatgttgggttaagtcccgcaacgagcgcaacccttatcttcagtagccagcatttcgggtgggcactctggagagactgccagggataacctggaggaaggtggggatgacgtcaaatcatcatgccccttatgagcagggctacacacgtgctacaatggcgtaaacaaagggaagcgaacctgtgagggtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagtcagtgactcaaccgtaaggagagagctgccgaaggtgggaccgataactggggtgaagtcgtaacaaggtagccgtatcggaaggtgcggctggatcacctccttt

SEQ ID NO: 26 - DNA, 1536 bp, Artificial Sequence
  Description of Artificial Sequence Synthetic polynucleotide
ttccttagaaaggaggtgatccagccgcaccttccgatacggctaccttgttacgacttcaccccagttatcaaacctgccttcggcggctccttctttcgttaggtcaccgacttcgggcattttcgactcccatggtgtgacgggcggtgtgtacaagacccgggaacgtattcaccgcagcattctgatctgcgattactagcgattccagcttcatgtagtcgagttgcagactacaatccgaactgagacgttatttttgtgatttgcttggcctcacgacttcgcttcactttgtttacgccattgtagcacgtgtgtagcccaagtcataaggggcatgatgatttgacgtcatccccaccttcctccaggttatccctggcagtctccctagagtgcccatcttactgctggctactaaggataggggttgcgctcgttgcgggacttaacccaacatctcacgacacgagctgacgacaaccatgcaccacctgtctccactgtcccgaaggaaaggacacattactgtccggtcagtgggatgtcaagacttggtaaggttcttcgcgttgcttcgaattaaaccacatgctccaccgcttgtgcgggtccccgtcaattcctttgagtttcattcttgcgaacgtactccccaggtggaatacttattgcgtttgctgcggcaccgaagcccttatgggccccgacacctagtattcatcgtttacggcgtggactaccagggtatctaatcctgtttgctccccacgctttcgagcctcagtgtcagttacagtccagtgagccgccttcgccactggtgttcctcctaatatctacgcatttcaccgctacactaggaattccactcacccctcctgcactccagccttacagtttcaaaagcagttccggggttgagccccggattttcacttctgacttgcatggccacctacactccctttacacccagtaaatccggataacgcttgctccatacgtattaccgcggctgctggcacgtatttagccggagcttcttagtcaggtaccgtcactatcttccctgctgatagagctttacataacgaattacttcttcactcacgcggcgtcgctgcatcagagtttcctccattgtgcaatattccccactgctgcctcccgtaggagtctgggccgtgtctcagtcccaatgtggccggtcaccctctcaggtcggctactgatcgtcgccttggtgggctgttatctcaccaactagctaatcagacgcgggtccatcttataccaccggagtttttcacaccatgtcatgcaacattgtgcgcttatgcggtattaccagccgtttccagctgctatcccccagtacaaggcaggttacccacgcgttactcacccgtccgccactcagtcataaagaacttcaaaccgaagtaatccgttctaaatgcttcgttcgacttgcatgtgttaagcacgccgccagcgttcatcctgagccaggatcaaactctcata

SEQ ID NO: 27 - DNA, 1275 bp, Artificial Sequence
  Description of Artificial Sequence Synthetic polynucleotide
  modified_base (204)..(204): a, c, t, g, unknown or other
  modified_base (207)..(207): a, c, t, g, unknown or other
ccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgncgncgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctccttt

SEQ ID NO: 28 - DNA, 1533 bp, Artificial Sequence
  Description of Artificial Sequence Synthetic polynucleotide
ccttagaaaggaggtgatccagccgcaccttccgatacggctaccttgttacgacttcaccccagttatcggtcccaccttcggcagctccctccttgcggttgggtcactgacttcgggcgttaccaactcccatggtgtgacgggcggtgtgtacaagacccgggaacgtattcaccgcgacattctgattcgcgattactagcgattccagcttcatgtagtcgagttgcagactacaatccgaactgagacgttatttttgagatttgcttaccctcgcaggctcgcttccctttgtttacgccattgtagcacgtgtgtagccctgctcataaggggcatgatgatttgacgtcatccccaccttcctccaggttatccctggcagtctctctagagtgcccggccrwaccgctggctactaaagataggggttgcgctcgttgcgggacttaacccaacatctcacgacacgagctgacgacaaccatgcaccacctgtcatccctgtcccgaaggaaaggcaacattacttgccggtcagggagatgtcaagagcaggtaaggttcttcgcgttgcttcgaattaaaccacatgctccaccgcttgtgcgggtccccgtcaattcctttgagtttcattcttgcgaacgtactccccaggtggactacttattgcgttggctgcggcaccgaatagctctgctacccgacacctagtagtcatcgtttacggcgtggactaccagggtatctaatcctgtttgctccccacgctttcgagcctcaacgtcagtcatcgtccagcaagccgccttcgccactggtgttcctcctaatatctacgcatttcaccgctacactaggaattccacttgcctctccgacactctagttcgacagtttccaatgcagtcccagggttgagccctggcctttcacatcagacttgccataccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcagggtttcccccattgtgcaatattccccactgctgcctcccgtaggagtttgggccgtgtctcagtcccaatgtggccggtcaccctctcaggtcggctactgatcgtcgccttggtaggccgttaccccaccaacyagctaatcagacgcgggtccatctcataccaccggagtttttacccctgcaccatgcggtgctgtggtcttatgcggtattagcagycatttctaactgttatccccctgtatgaggcaggttacccacgcgttactcacccgtccgccactcagtcacaaaagtcttcatccgaagaatcaaacttaagtgcttcgttcgacttgcatgtgttaagcacgccgccagcgttcatcctgagccaggatcaaactctcgt

SEQ ID NO: 29 - DNA, 1533 bp, Artificial Sequence
  Description of Artificial Sequence Synthetic polynucleotide
ttaaacgagagtttgatcctggctcaggatgaacgctggcggcgtgcttaacacatgcaagtcgagcgaagcacttaagttcgattcttcggatgaagacttttgtgactgagcggcggacgggtgagtaacgcgtgggtaacctgcctcatacagggggataacagttagaaatggctgctaataccgcataagaccacagtactgcatggtacagtggtaaaaactccggtggtatgagatggacccgcgtctgattaggtagttggtgaggtaacggcccaccaagccgacgatcagtagccgacctgagagggtgaccggccacattgggactgagacacggcccagactcctacgggaggcagcagtggggaatattgcacaatgggcgaaagcctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggctgtgcaagtctgaagtgaaaggcatgggctcaacctgtggactgctttggaaactgtgcagctagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgactgctaggtgtcgggtagcaaagctattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccttatcttcagtagccagcatttaagatgggcactctggagagactgccagggataacctggaggaaggtggggatgacgtcaaatcatcatgccccttatgaccagggctacacacgtgctacaatggcgtaaacaaagggaagcagagccgcgaggccgagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgaaaggagggagctgccgaaggtgggaccgataactggggtgaagtcgtaacaaggtagccgtatcggaaggtgcggctggatcacctcctttct

SEQ ID NO: 30 - DNA, 1444 bp, Artificial Sequence
  Description of Artificial Sequence Synthetic polynucleotide
  modified_base (377)..(377): a, c, t, g, unknown or other
  modified_base (905)..(905): a, c, t, g, unknown or other
  modified_base (909)..(909): a, c, t, g, unknown or other
  modified_base (913)..(913): a, c, t, g, unknown or other
  modified_base (941)..(941): a, c, t, g, unknown or other
acgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttnctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacngaantttncagagatgagaatgtgccttcgggaacngtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 31 - DNA, 1529 bp, Artificial Sequence
  Description of Artificial Sequence Synthetic polynucleotide
atgagagtttgatcctggctcaggatgaacgctggcggcgtgcttaacacatgcaagtcgaacgaagcgccttatttgattttcttcggaactgaagatttggtgactgagtggcggacgggtgagtaacgcgtgggtaacctgccctgtacagggggataacaatcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatggggggaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcaataagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagtgacaggtggtgcatggttgtcgtcagctcgtgtcgtgagatgttgggttaagtcccgcaacgagcgcaacccctatctttagtagccagcatttaaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctccttt

SEQ ID NO: 32 - DNA, 1260 bp, Artificial Sequence
  Description of Artificial Sequence Synthetic polynucleotide
gttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcaaagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaaatcttgacatccctctgaccggtctttaatcggaccttctcttcggagcagaggtgacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagatcgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggtagccgtatcggaaggtgcggctggatcac

SEQ ID NO: 33 - DNA, 1534 bp, Artificial Sequence
  Description of Artificial Sequence Synthetic polynucleotide
aacgagagtttgatcctggctcaggatgaacgctggcggcgtgcctaacacatgcaagtcgaacgaagcgcttccgcctgattttcttcggagatgaaggcggctgcgactgagtggcggacgggtgagtaacgcgtgggcaacctgccttgcactgggggataacagccagaaatggctgctaataccgcataagaccgaagcgccgcatggcgcagcggccaaagccccggcggtgcaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcattccggatgggcactctggagagactgccagggacaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagccggtgacccaacccgcaagggagggagccgtcgaaggtgggaccgataactggggtgaagtcgtaacaaggtagccgtatcggaaggtgcggctggatcacctccttt

SEQ ID NO: 34 - DNA, 1527 bp, Artificial Sequence
  Description of Artificial Sequence Synthetic polynucleotide
tttagagagtttgatcctggctcaggatgaacgctggcggcgtgcctaacacatgcaagtcgaacggagtcgttttggaaaatccttcgggattggaattctcgacttagtggcggacgggtgagtaacgcgtgagcaatctgcctttaagagggggataacagtcggaaacggctgctaataccgcataaagcattgaattcgcatgttttcgatgccaaaggagcaatccgcttttagatgagctcgcgtctgattagctagttggtggggtaacggcccaccaaggcgacgatcagtagccggactgagaggttgaacggccacattgggactgagacacggcccagactcctacgggaggcagcagtggggaatattgcgcaatgggggaaaccctgacgcagcaacgccgcgtgattgaagaaggccttcgggttgtaaagatctttaatcagggacgaaaaatgacggtacctgaagaataagctccggctaactacgtgccagcagccgcggtaatacgtagggagcaagcgttatccggatttactgggtgtaaagggcgcgcaggcgggccggcaagttggaagtgaaatccgggggcttaacccccgaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggatactaggtgtgggaggtattgaccccttccgtgccgcagttaacacaataagtatcccacctggggagtacggccgcaaggttgaaactcaaaggaattgacgggggcccgcacaagcagtggagtatgtggtttaattcgaagcaacgcgaagaaccttaccaggccttgacatcccgatgaccggcttagagataagccttctcttcggagcatcggtgacaggtggtgcatggttgtcgtcagctcgtgtcgtgagatgttgggttaagtcccgcaacgagcgcaacccttacggttagttgatacgcaagatcactctagccggactgccgttgacaaaacggaggaaggtggggacgacgtcaaatcatcatgccccttatggcctgggctacacacgtactacaatggcagtcatacagagggaagcaaaatcgcgaggtggagcaaatccctaaaagctgtcccagttcagattgcaggctgcaacccgcctgcatgaagtcggaattgctagtaatcgcggatcagcatgccgcggtgaatacgttcccgggccttgtacacaccgcccgtcacaccatgagagccgtcaatacccgaagtccgtagcctaaccgcaaggagggcgcggccgaaggtaggggtggtaattagggtgaagtcgtaacaaggtagccgtatcggaaggtgcggctggatcacctccttt

SEQ ID NO: 35 - DNA, 1513 bp, Artificial Sequence
  Description of Artificial Sequence Synthetic polynucleotide
agaaaggaggtgatccagccgcaggttctcctacggctaccttgttacgacttcaccccaatcaccagttttaccttcggcggcgtcctccttgcggttagactaccgacttcgggtccccccggctctcatggtgtgacgggcggtgtgtacaaggcccgggaacgtattcaccgtggcatgctgatccacgattactagcaattccgacttcgtgcaggcgagttgcagcctgcagtccgaactgggacgttgtttctgagttttgctccacctcgcggtcttgcttctctttgtttaacgccattgtagtacgtgtgtagcccaagtcataaagggcatgatgatttgacgtcatccccaccttcctccgttttgtcaacggcagtcctgccagagtcctcttgcgtagtaactgacagtaagggttgcgctcgttgcgggacttaacccaacatctcacgacacgagctgacgacaaccatgcaccacctgtctccttgctccgaagagaaaacatatttctatgtgcgtcgcaggatgtcaagacttggtaaggttcttcgcgttgcgtcgaattaaaccacatactccactgcttgtgcgggcccccgtcaattcctttgagtttcaaccttgcggtcgtactccccaggtggattacttattgtgttaactgcggcactgaaggggtcaatcctccaacacctagtaatcatcgtttacggtgtggactaccagggtatctaatcctgtttgctacccacactttcgagcctcagcgtcagttggtgcccagtaggccgccttcgccactggtgttcctcccgatatctacgcattccaccgctacaccgggaattccgcctacctctgcactactcaagaaaaacagttttgaaagcagtttatgggttgagcccatagatttcacttccaacttgtcttcccgcctgcgctccctttacacccagtaattccggacaacgcttgtgacctacgttttaccgcggctgctggcacgtagttagccgtcacttccttgttgagtaccgtcattatcttcctcaacaacaggagtttacaatccgaagaccttcttcctccacgcggcgtcgctgcatcagggtttcccccattgtgcaatattccccactgctgcctcccgtaggagtctgggccgtgtctcagtcccaatgtggccgttcaacctctcagtccggctaccgatcgttgccttggtgggccattacctcaccaactagctaatcggacgcgaggccatctcaaagcggattgctccttttccctctggtcgatgccgacctgtgggcttatgcggtattagcagtcgtttccaactgttgtccccctctttgaggcaggttcctcacgcgttactcacccgttcgccactcgctygagaaagcaagctctctctcgctcgttcgacttgcatgtgttaggcgcgccgccagcgttcgtcctgagccaggatcaaactctttataaa

SEQ ID NO: 36 - DNA, 1584 bp, Artificial Sequence
  Description of Artificial Sequence Synthetic polynucleotide
ccgtgtctcagttccaatgtgggggaccttcctctcagaacccctatccatcgttgtcttggtgggccgttaccccgccaacaaactaatggaacgcatccccatcgataaccgaaattctttaatagtaaaaccatgcggttttaatataccatcggatattaatctttctttcgaaaggctatccccgagttatcggcaggttggatacgtgttactcacccgtgcgccggtcgccatctttagtttgcaagcaaactaaaatgctgcccctcgacttgcatgtgttaagcctgtagctagcgttcatcctgagctattaaagaatttcggttatcgatggggatgcgttccattagtttgttggcggggtaacggcccaccaagacaacgatggataggggttctgagaggaaggtcccccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaaggcaaatgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggct

SEQ ID NO: 37 - DNA, 1522 bp, Artificial Sequence
  Description of Artificial Sequence Synthetic polynucleotide
atgagagtttgatcctggctcaggatgaacgctggcggcgtgcttaacacatgcaagtcgaacggacgatgaagagcttgcttttcagagttagtggcggacgggtgagtaacgcgtgggtaacctgcctcatacagggggatagcagctggaaacggctgataaaaccgcataagcgcacagcatcgcatgatgcagtgtgaaaaactccggtggtatgagatggacccgcgtctgattagctggttggtgaggtaacggcccaccaaggcgacgatcagtagccggcctgagagggtgaccggccacattgggactgagacacggcccaaactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtaggcggcggagcaagtcagaagtgaaagcccggggctcaaccccgggacggcttttgaaactgccctgcttgatttcaggagaggtaagcggaattcctagtgtagcggtgaaatgcgtagatattaggaggaacaccagtggcgaaggcggcttactggactgacaatgacgctgaggctcgaaagcgtggggagcaaacaggattagataccctggtagtccacgccgtaaacgatgaatactaggtgtcggggctcataagagcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaggccttgacatcccggtgaccgtcccgtaatggggacctctcttcggagcaccggtgacaggtggtgcatggttgtcgtcagctcgtgtcgtgagatgttgggttaagtcccgcaacgagcgcaacccctatgttcagtagccagcaggtaaagctgggcactctggacagactgccggggataacccggaggaaggcggggatgacgtcaaatcatcatgccccttacggcctgggctacacacgtgctacaatggcgtaaacaaagggaagcgagagggtgacctggagcgaatcccaaaaataacgtcccagttcggactgtagtctgcaacccgactacacgaagctggaatcgctagtaatcgcgaatcagcatgtcgcggtgaatacgttcccgggtcttgtacacaccgcccgtcacaccatgggagttggaaatgcccgaagtcagtgacctaaccgcaagggaggagctgccgaaggtggagccgatgactggggtgaagtcgtaacaaggtagccgtatcggaaggtgcggctggatcacctcctttctaaggaa

SEQ ID NO: 38 - DNA, 1524 bp, Artificial Sequence
  Description of Artificial Sequence Synthetic polynucleotide
tgtggagggttcgattctggctcaggatgaacgctggcggcgtgcttaacacatgcaagtcgaacgggatcccaggagcttgctcctgggtgagagtggcgaacgggtgagtaatgcgtgaccgacctgccccatacaccggaatagctcctggaaacgggtggtaatgccggatgctccagttgaccgcatggtcctctgggaaagcttttgcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacaccgcgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaaccttttggggggagccgtctaaggtgagactcgtgattgggactaagtcgtaacaaggtagccgtaccggaaggtgcggctggatcacctccttt

SEQ ID NO: 39 - DNA, 1503 bp, Artificial Sequence
  Description of Artificial Sequence Synthetic polynucleotide
cggagagttcgatcctggctcaggatgaacgctggcggcgcgcctaacacatgcaagtcgaacggcacccacctccgggtggaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtggggggacgatccccccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatatgggtgaagcgggggagaccccgtggccgagaggagcccatacaggtggtgcatggctgtcgtcagctcgtgtcgtgagatgttgggttaagtcccgcaacgagcgcaacccccgccgcgtgttgccatcgggtgatgccgggaacccacgcgggaccgccgccgtcaaggcggaggagggcggggacgacgtcaagtcatcatgccccttatgccctgggctgcacacgtgctacaatggccggtacagagggatgccaccccgcgagggggagcggatcccggaaagccggccccagttcggattgggggctgcaacccgcccccatgaagtcggagttgctagtaatcgcggatcagcatgccgcggtgaatgcgttcccgggccttgtacacaccgcccgtcacaccacccgagtcgtctgcacccgaagtcgccggcccaaccgtcaaggggggaggcgccgaaggtgtggagggtgaggggggtgaagtcgtaacaaggtagccgtaccggaaggtgcggctggatcacctccttt

SEQ ID NO: 40 - DNA, 1490 bp, Artificial Sequence
  Description of Artificial Sequence Synthetic polynucleotide
  modified_base (18)..(18): a, c, t, g, unknown or other
  modified_base (26)..(26): a, c, t, g, unknown or other
gacttcaccccaatcatnggccccanttagacagctgactcctaaaaggttatctcaccggcttcgggtgttaccaactttcgtggtgtgacgggcggtgtgtacaaggcccgggaacgtattcaccgcagtatgctgacctgcgattactagcgattccaacttcacgtaggcgggttgcagcctacgatccgaactggggtcgggtttctgggatttgctccacctcgcggtttcgctgccctttgttgccgaccattgtagtacgtgtgtagcccaagacataaggggcatgatgacttgacgtcatccccgccttcctccaagttatccctggcagtctcctatgagtccccgcctttacgcgctggtaacataggataggggttgcgctcgttgcgggacttaacccaacatctcacgacacgagctgacgacagccatgcaccacctgttttcgtgtccccgaagggagggacctatctctaggtctttcactcaatgtcaagccttggtaaggttcttcgcgttgcgtcgaattaaaccacatactccaccgcttgtgcgggcccccgtcaattcctttgagtttcaatcttgcgatcgtagtccccaggcgggatacttattgcgttaactccggcacagaaggggtcgatacctcctacacctagtatccatcgtttacggccaggactaccggggtatctaatcccgtttgctaccctggctttcgcatctcagcgtcagacacagtccagaaaggcgccttcgccactggtgttcctcccaatatctacgcatttcaccgctacactgggaattcccctttcctctcctgcactcaagacttccagtatccaacgccatacggggttaagccccgcattttcacgtcagacttaaaagcccgcctacatgctctttacgcccaataattccggacaacgcttgccacctacgtattaccgcggctgctggcacgtagttagccgtggcttcctcgttaggtaccgtcaacaccatgacctgttcgaacacggtgctttcgtccctaacaacagagttttacaatccgaagaccttcatcactcacgcggcgttgctccgtcagactttcgtccattgcggaagattccccactgctgcctcccgtaggagtttgggccgtgtctcagtcccaatgtggccgttcatcctctcagaccggctactgatcatcgccttggtgagccgttaccccaccaactagctaatcagacgcgggcccatcttccagcgatagcttgcaagcagaggccatctttcctccctcctccatgcggaggagggagcacattcggtattagcatccctttcggaatgttgtccccaactggagggcaggttgcccacgcgttactcacccgttcgccactaagaacttaccgaaataagttctccgttcgacttgcatgtgttaagcacgccgccagcgttcgtcctgagcc

SEQ ID NO: 41 - DNA, 674 bp, Artificial Sequence
  Description of Artificial Sequence Synthetic polynucleotide
  modified_base (623)..(623): a, c, t, g, unknown or other
aggagtgtttccactccggtcagactacgttcaaacccgggtaaggttcctcgcgtatcatcgaattaaaccacatgttcctccgcttgtgcgggcccccgtcaattcctttgagtttcaccgttgccggcgtactccccaggtggattacttaacgctttcgctgtagagcttacattgtatcgcaaactcctagtaatcatcgtttactgcgtggactaccagggtatctaatcctgtttgatccccacgctttcgtgcttcagtgtcagttatggtttagtaagctgccttcgcaatcggagttctgcgtgatatctatgcatttcaccgctacaccacgcattccgcctacctcaaacacactcaagtaacccagtttcaacggcaattttatggttgagccacaaactttcaccgctgacttaaatcaccacctacgcaccctttaaacccaataaatccggataacgctcgcatcctccgtattaccgcggctgctggcacggagttagccgatgcttattcatagggtacatacaaaaaaggacacgtcctccactttattcccctataaaagaagtttacaaaccatagatccttcttccttcacgcgacttggntggttcagcctctcggccattgaccaatattcctcactgctgcctcccgta

SEQ ID NO: 42 - DNA, 638 bp, Artificial Sequence
  Description of Artificial Sequence Synthetic polynucleotide
  modified_base (6)..(6): a, c, t, g, unknown or other
tcaaanggatgtcaagacctggtaaggttcttcgcgttgcttcgaattaaaccacatgctccaccgcttgtgcgggtccccgtcaattcctttgagtttcattcttgcgaacgtactccccaggtggaatacttactgcgttagcggcggcaccgaagcctatacggccccgacacctagtattcatcgtttacggcgtggactaccagggtatctaatcctgtttgctccccacgctttcgtgcctcagtgtcagtaacagtccagcaggccgccttcgccactggtgttcctcctaatatctacgcatttcaccgctacactaggaattccgcctgcctctcctgtactctagccgagcagtttcaaatgcagctccggggttgagccccggcctttcacatctgacttgcactgccacctacgcaccctttacacccagtaaatccggataacgcttgctccatacgtattaccgcggctgctggcacgtatttagccggagcttcttagtcaggtaccgtcattatcttccctgctgatagagctttacataccgaaatacttcttcactcacgcggcgttgctgcatcagggtttcccccattgtgcaatattccccactgctgcctcccgta

SEQ ID NO: 43 - DNA, 702 bp, Artificial Sequence
  Description of Artificial Sequence Synthetic polynucleotide
  modified_base (31)..(31): a, c, t, g, unknown or other
  modified_base (34)..(34): a, c, t, g, unknown or other
  modified_base (649)..(649): a, c, t, g, unknown or other
  modified_base (653)..(653): a, c, t, g, unknown or other
  modified_base (655)..(655): a, c, t, g, unknown or other
  modified_base (662)..(663): a, c, t, g, unknown or other
agcacctcgcaacggctattgctagaaaagntgnttccacctcggtccgaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacactgtatcgcaaacagctagtgatcatcgtttactgcgtggactaccagggtatctaatcctgtttgatccccacgctttcgtgcatcagcgtcagtcatggcttggcaggctgccttcgcaatcggggttctgcgtgatatctaagcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacaggacacgtcccgcactttattcccttataaaagaggtttacgatccatagaaccttcatccctcacgcgacttggntggntncaggctnnacgcccattgaccaatattcctcactgctgcctcccgta

SEQ ID NO: 44 - DNA, 705 bp, Artificial Sequence
  Description of Artificial Sequence Synthetic polynucleotide
  modified_base (5)..(5): a, c, t, g, unknown or other
  modified_base (9)..(9): a, c, t, g, unknown or other
  modified_base (162)..(163): a, c, t, g, unknown or other
  modified_base (165)..(165): a, c, t, g, unknown or other
  modified_base (574)..(574): a, c, t, g, unknown or other
  modified_base (584)..(584): a, c, t, g, unknown or other
ctgtnttcntgtccccgaagggaaagctccatctctggagcggtcattcaatgtcaagccttggtaaggttcttcgcgttgcgtcgaattaaaccacatactccaccgcttgtgcgggcccccgtcaattcctttgagtttcatccttgcggacgtactccnnangcggggtacttattgcgttaactccggcacagaaggggtcgatacctcctacacctagtacccatcgtttacggccaggactaccggggtatctaatcccgttcgctaccctggctttcgcatctcagcgtcagacacagtccagaaaggcgccttcgccactggtgttcctcccaatatctacgcatttcaccgctacactgggaattcccctttcctctcctgcactcaagtcttccagtatccagagccatacggggttgagccccgcattttcactccagacttaaaaaaccgcctacatgctctttacgcccaataattccggacaacgctcgccacctacgtattaccgcggctgctggcacgtagttagccgtggcttattcgtttactaccgtcattacanatcattattnacaatctgcacattcgtcataaacaaaagagctttacggaacgaattccttcatcactcacgcggcattgctccgtcaggctttcgcccattgcggaagattccccactgctgcctccgta

SEQ ID NO: 45 - DNA, 650 bp, Artificial Sequence
  Description of Artificial Sequence Synthetic polynucleotide
  modified_base (10)..(10): a, c, t, g, unknown or other
  modified_base (17)..(17): a, c, t, g, unknown or other
  modified_base (559)..(559): a, c, t, g, unknown or other
attactcagncgtcacngggatgtcaagagcaggtaaggttcttcgcgttgcttcgaattaaaccacatgctccaccgcttgtgcgggtccccgtcaattcctttgagtttcattcttgcgaacgtactccccaggtggactacttattgcgttggctgcggcaccgaatggctttgccacccgacacctagtagtcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacctctccggcactccagatcaacagtttccaatgcagtcccggggttgagccccgggttttcacatcagacttgctgctccgtctacgctccctttacacccagtaaatccggataacgcttgccccctacgtattaccgcggctgctggcacgtagttagccggggcttcttagtcaggtaccgtcattttcttccctgctgataganctttacataccgaaatacttcttcgctcacgcggcgtcgctgcatcagggtttcccccattgtgcaatattccccactgctgcctcccgta

SEQ ID NO: 46 - DNA, 683 bp, Artificial Sequence
  Description of Artificial Sequence Synthetic polynucleotide
ctgtctcaactttccccgaagggcacctaatgcatctctgcttcgttagttggatgtcaagacctggtaaggttcttcgcgttgcttcgaattaatccacatactccactgcttgtgcgggcccccgtcaattcctttgagtttcaaccttgcggtcgtactccccaggtggattacttattgtgttaactccggcacggagggggtcagaccccccacacctagtaatcatcgtttacggcatggactaccagggtatctaatcctgtttgctacccatgctttcgtgcctcagcgtcagttaaagcccagtaggccgccttcgccactggtgttcctcccgatctctacgcatttcaccgctacaccgggaattccgcctacctctacttcactcaagctctacagtttcaaaagcagttcatgggttaagcccatggatttcacttctgacttgcagagccgcctacgcaccctttacacccagtaaatccggacaacgctcgctccctacgtattaccgcggctgctggcacgtagttagccggagcttcctcctctgctaccgtcactatcttcacagaggacagaggtttacaatccgaaaaccgtcttccctcacgcggcgttgctggttcagggtttcccccattgaccaatatccctcactgctgcctcccgta

SEQ ID NO: 47 - DNA, 651 bp, Artificial Sequence
  Description of Artificial Sequence Synthetic polynucleotide
  modified_base (542)..(542): a, c, t, g, unknown or other
  modified_base (630)..(630): a, c, t, g, unknown or other
atgtcaagacctggtaaggttcttcgcgttgcttcgaattaaaccacatgctccaccgcttgtgcgggcccccgtcaattcctttgagtttcattcttgcgaacgtactactcaggcggagtacttattgcgttaactgcagcactgagatttgactcccaacacttagtactcatcgtttacggcgtggactactagggtatctaatcctatttgctccccacgctttcgggactgagcgtcagttatgcgccagatcgtcgccttcgccactggtgttcctccatatatctacgcatttcaccgctacacatggaattccacgatcctctcacacactctagctctacggtttccatggcttaccgaagttaagcttcgatctttcaccacagacccttagtgccgcctgctccctctttacgcccaataattccggataacgctcgccacctacgtattaccgcggctgctggcacgtagttagccgtggctttcttatagagtaccgtcacttggatatcattccctatatccaccgntcttcctctatgacagaagtttacataacgaattacttcttccttcacgcggcgttgctcggtcagggtttcccccattgccgaaaantccctactgctgcctcccgta

SEQ ID NO: 48 - DNA, 644 bp, Artificial Sequence
  Description of Artificial Sequence Synthetic polynucleotide
  modified_base (7)..(7): a, c, t, g, unknown or other
ctgtcanacgggatgtcaagacttggtaaggttcttcgcgttgcttcgaattaaaccacatgctccaccgcttgtgcgggtccccgtcaattcctttgagtttcattcttgcgaacgtactccccaggtggaatacttattgcgtttgctgcggcaccgaagagcaatgctccccgacacctagtattcatcgtttacggcgtggactaccagggtatctaatcctgtttgctccccacgctttcgagcctcagcgtcagtaatcgtccagtaagccgccttcgccactggtgttcctcctaatatctacgcatttcaccgctacactaggaattccacttacccctccgacactctagtccgacagtttccaatgcagtaccggggttgagccccgggctttcacatcagacttgccgtaccgcctgcgctccctttacacccagtaaatccggataacgcttgcaccatacgtattaccgcggctgctggcacgtatttagccggtgcttcttagtcaggtaccgtcatttcttcttccctgctgatagagctttacataccgaaatacttcttcgctcacgcggcgtcgctgcatcagggtttcccccattgtgcaatattccccactgctgcctcccgta

SEQ ID NO: 49 - DNA, 437 bp, Artificial Sequence
  Description of Artificial Sequence Synthetic polynucleotide
tcttgcgaccgtactccccaggcggcacgcttaacgcgttagctccggcacgcagggggtcgattccccgcacaccaagcgtgcaccgtttactgccaggactacaggggtatctaatccctttcgctcccctggccttcgtgcctcagcgtcagttaatgtccaggaacccgccttcgccacgagtgttcctctcgatatctacgcatttcactgctacaccgagaattccggttccccctccattactctagtctcgcagtatcatgtgccgtccgcgggttgagcccgcgcctttcacacacgacttacgaaacagcctacgcacgctttacgcccagtgattccgaacaacgcttgagacctctgtattaccgcggctgctggcacagagttagccgtctcttcctcttgtggtactatctttttaatttgct

SEQ ID NO: 50 - DNA, 652 bp, Artificial Sequence
  Description of Artificial Sequence Synthetic polynucleotide
  modified_base (110)..(116): a, c, t, g, unknown or other
  modified_base (152)..(156): a, c, t, g, unknown or other
tccggttaaggaacggtcagagggatgtcaagacttggtaaggttcttcgcgttgcttcgaattaaaccacatgctccaccgcttgtgcgggtccccgtcaattcctttnnnnnnnattcttgcgaacgtactccccaggtggaatacttannnnntttgcggcggcaccgaagagctttgctccccaacacctagtattcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacctctccggcactcaagactaacagtttccaatgcagtccaggggttgagcccccgcctttcacatcagacttgccagtccgtctacgctccctttacacccagtaaatccggataacgcttgccccctacgtattaccgcggctgctggcacgtagttagccggggcttcttagtcaggtaccgtcactatcttccctgctgatagaagtttacataccgagatacttcttccttcacgcggcgtcgctgcatcagggtttcccccattgtgcaatattccccactgctgcctcccg

SEQ ID NO: 51 - DNA, 681 bp, Artificial Sequence
  Description of Artificial Sequence Synthetic polynucleotide
  modified_base (20)..(20): a, c, t, g, unknown or other
  modified_base (26)..(26): a, c, t, g, unknown or other
ctgtcaccgatgctccgaanagaaantctatctctagagcggtcatcgggatgtcaagccttggtaaggttcttcgcgttgcttcgaattaaaccacatgctccaccgcttgtgcgggcccccgtcaattcctttgagtttcaaccttgcggtcgtactccccaggtggagtgcttattgcgttagctgcggcaccgaggattcctccccgacacctagcactcatcgtttacggcgtggactaccagggtatctaatcctgtttgctccccacgctttcgagcctcagcgtcagtttcagtccagaaagccgccttcgccactggtgttcttcctaatatctacgcatttcaccgctacactaggaattccgctttcctctcctgtactctagctcgccagttccaaatgcaatccgcgaggttaagcctcgggctttcacatctggcttggcgtgccgcctacgctccctttacacccagtaattccggataacgcttgccccctacgtattaccgcggctgctggcacgtagttagccggggcttcttattcaggtaccgtcactttcttcgtccctgttgatagaagtttacgatccgaaaaccttcttccttcacgcggcgttgctgcatcagggtttcccccattgtgcaatattccccactgctgcctcccgta

SEQ ID NO: 52 - DNA, 616 bp, Artificial Sequence
  Description of Artificial Sequence Synthetic polynucleotide
  modified_base (10)..(10): a, c, t, g, unknown or other
  modified_base (26)..(26): a, c, t, g, unknown or other
  modified_base (569)..(569): a, c, t, g, unknown or other
  modified_base (579)..(581): a, c, t, g, unknown or other
  modified_base (584)..(586): a, c, t, g, unknown or other
  modified_base (595)..(595): a, c, t, g, unknown or other
  modified_base (597)..(597): a, c, t, g, unknown or other
  modified_base (600)..(600): a, c, t, g, unknown or other
  modified_base (603)..(603): a, c, t, g, unknown or other
  modified_base (610)..(610): a, c, t, g, unknown or other
cctgtctccnccgtccgtagaaaggnccgattaaggaccggtcggagggatgtcaagccctggtaaggttcttcgcgttgcttcgaattaaaccacatgctccgctgcttgtgcgggtccccgtcaattcctttgagttttacacttgcgtgcgtactccccaggcggagcacttaatgcgttaactccggcaccgaactcttgcgagcccgacacctagtgctcatcgtttacggcgtggactaccagggtatctaatcctgtttgctccccacgctttcgtatctcagtgtcagttactgtccagaaagccgccttcgccactggtgttcctcctaatatctacgcatttcaccgctacactaggaattccgctttcctctccagcactcaagtctctcagttcacggggcgaacaatggttaagccattgccttataccctgcgcttaagaaaccacctacatactctttacgcccaataattccggataacgcttgccccctacgtattaccgcggctgctggcacgtagttagccggggcttcctccttgggtaccgtcatttnttcttccccnnngannnaactttacnanccnaangccttcntcgctc

SEQ ID NO: 53 - DNA, 649 bp, Artificial Sequence
  Description of Artificial Sequence Synthetic polynucleotide
gcaacattacttgccggtcagggagatgtcaagagcaggtaaggttcttcgcgttgcttcgaattaaaccacatgctccaccgcttgtgcgggtccccgtcaattcctttgagtttcattcttgcgaacgtactccccaggtggactacttattgcgtttgctgcggcaccgaacagctttgctgcccgacacctagtagtcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacctctccggtactctagattgacagtttccaatgcagtcccggggttgagccccgggttttcacatcagacttgccactccgtctacgctccctttacacccagtaaatccggataacgcttgcaccatacgtattaccgcggctgctggcacgtatttagccggtgcttcttagtcaggtaccgtcattttcttccctgctgatagagctttacataccgaaatacttcatcgctcacgcggcgtcgctgcatcagggtttcccccattgtgcaatattccccactgctgcct

SEQ ID NO: 54 - DNA, 674 bp, Artificial Sequence
  Description of Artificial Sequence Synthetic polynucleotide
ctgtcactcctgctccgaagagaaggtacggttaagtaccggtcagaaggatgtcaagacttggtaaggttcttcgcgttgcttcgaattaaaccacatgctccaccgcttgtgcgggtccccgtcaattcctttgagtttcattcttgcgaacgtactccccaggtggaatacttactgcgtttgcgacggcaccgagaagcaatgcttcccaacacctagtattcatcgtttacggcgtggactaccagggtatctaatcctgtttgctccccacgctttcgagcctcagcgtcagttatcgtccagtaagccgccttcgccactggtgttcctcctaatatctacgcatttcaccgctacactaggaattccgcttacccctccgacactctagtacgacagtttccaatgcagtaccggggttgagccccgggctttcacatcagacttgccgcaccgcctgcgctccctttacacccagtaaatccggataacgcttgcaccatacgtattaccgcggctgctggcacgtatttagccggtgcttcttagtcaggtaccgtcattatcttccctgctgatagagctttacataccgaaatacttcttcgctcacgcggcgtcgctgcatcaggctttcgcccattgtgcaatattccccactgctgcct

SEQ ID NO: 55 - DNA, 693 bp, Artificial Sequence
  Description of Artificial Sequence Synthetic polynucleotide
agcaccttcacagcggcccgaaggctgtaatgtttccactacattccgctgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgctgactgtgtatcgccaacagcgagtattcatcgtttactgtgtggactaccagggtatctaatcctgtttgatacccacactttcgagcatcagcgtcagttacagtccagtaagctgccttcgcaatcggagttcttcgtgatatctaagcatttcaccgctacaccacgaattccgcctacctatgctgcactcaaggtacccagtatcaactgcaattttacggttgagccgcaaactttcacaactgacttaagcacccgcctacgctccctttaaacccaataaatccggataacgctcggatcctccgtattaccgcggctgctggcacggagttagccgatccttattcatacggtacatacaaaggagtatgcatactccactttattcccgtataaaagaagtttacaacccatagggcagtcatccttcacgctacttggctggttcagactctcgtccattgaccaatattcctcactgctgcct

SEQ ID NO: 56 - DNA, 661 bp, Artificial Sequence
  Description of Artificial Sequence Synthetic polynucleotide
ccgaagaaaggacacattactgtccggtcaccgggatgtcaagacttggtaaggttcttcgcgttgcttcgaattaaaccacatgctccaccgcttgtgcgggtccccgtcaattcctttgagtttcattcttgcgaacgtactccccaggtggaatacttaatgcgtttgcggcggcaccgaaaagctgtgctttccgacacctagtattcatcgtttacggcgtggactaccagggtatctaatcctgtttgctccccacgctttcgagcctcagcgtcagttatcgtccagtaagccgccttcgccactggtgttcctcctaatatctacgcatttcaccgctacactaggaattccacttacccctccgacactctagatgaccagtttccaatgcagtaccggggttgagccccgggctttcacatcagacttagccttccgcctgcgctccctttacacccagtaaatccggataacgcttgcaccatacgtattaccgcggctgctggcacgtatttagccggtgcttcttagtcaggtaccgtcatttcttcttccctgctgatagagctttacataccgaaatacttcttcgctcacgcggcgtcgctgcatcagggtttcccccattgtgcaatattccccactgctgcct

SEQ ID NO: 57 - DNA, 683 bp, Artificial Sequence
  Description of Artificial Sequence Synthetic polynucleotide
  modified_base (678)..(678): a, c, t, g, unknown or other
ctgtcaccggtgttccgaagaaagagtactttacatactttgtcaccgggatgtcaagatttggtaaggttcttcgcgttgcttcgaattaaaccacatgctccaccgcttgtgcgggtccccgtcaattcctttgagtttcattcttgcgaacgtactccccaggtggattacttattgcgtttgctgcggcaccgaagagtttaactccccgacacctagtaatcatcgtttacggcgtggactaccagggtatctaatcctgtttgctccccacgctttcgagcctcaacgtcagttacagtccagtaagccgccttcgccactggtgttcctcctaatatctacgcatttcaccgctacactaggaattccacttacctctcctgcactctagtttaccagtttccaaagcagttccggggttgagccccgggctttcacttcagacttggtctaccgtctacgctccctttacacccagtaaatccggataacgcttgccccctacgtattaccgcggctgctggcacgtagttagccggggcttcttagtcaggtaccgtcattgttttcttccctgctgatagagctttacataccgaaatacttcttcactcacgcggcgtcgctgcatcagggtttcccccattgtgcaatattccccactgctgcctnccgta

SEQ ID NO: 58 - DNA, 650 bp, Artificial Sequence
  Description of Artificial Sequence Synthetic polynucleotide
  modified_base (583)..(583): a, c, t, g, unknown or other
  modified_base (604)..(605): a, c, t, g, unknown or other
  modified_base (608)..(608): a, c, t, g, unknown or other
attccccctgcgtttgagcccgggtaaggttcctcgcgtatcatcgaattaaaccacatgttcctccgcttgtgcgggcccccgtcaattcctttgagtttcaccgttgccggcgtactccccaggtggaatacttaacgctttcgctgtaccgcttacattgtatcgcaaacagttagtattcatcgtttactgcgtggactaccagggtatctaatcctgttcgatacccacgctttcgtgcctcagcgtctgtttcaccctagcaggctgccttcgcaatcggggttctgcgtgatatctatgcatttcaccgctacaccacgcattccgcctgcctcttgtgcactctagctcgccagtttcaacggcacactccgggttgagcccggaaatttcaccgctgacttggcgtgccgcctacgcaccctttaaacccaataaatccggataacgcttgcatcctccgtattaccgcggctgctggcacggagttagccgatgctttttcttcggatacttgcaatacgctacacgtagcgtactttactctccgacaaaagaggtttacaacccgtagggncgtcttccctcacgcgacttnntgnttcagtctgccgaccattgaccaatattcctcactgctgcct

SEQ ID NO: 59 - DNA, 624 bp, Artificial Sequence
  Description of Artificial Sequence Synthetic polynucleotide
  modified_base (8)..(9): a, c, t, g, unknown or other
  modified_base (11)..(11): a, c, t, g, unknown or other
  modified_base (14)..(14): a, c, t, g, unknown or other
  modified_base (30)..(30): a, c, t, g, unknown or other
  modified_base (36)..(36): a, c, t, g, unknown or other
  modified_base (40)..(40): a, c, t, g, unknown or other
  modified_base (70)..(72): a, c, t, g, unknown or other
  modified_base (74)..(76): a, c, t, g, unknown or other
  modified_base (86)..(87): a, c, t, g, unknown or other
  modified_base (125)..(125): a, c, t, g, unknown or other
agacttgnnanggntcttcgcgttgcttcnaattanaccncatgctccaccgcttgtgcgggtccccatnnntnnntttaataaannttcttgcgaacgtactccccaggtggaatacttattgngtttgcggcggcaccgaagagctgtgctccccgacacctagtattcatcgtttacggcgtggactaccagggtatctaatcctgtttgctccccacgctttcgagcctcaacgtcagttactgtccagtaagccgccttcgccactggtgttcctcctaatatctacgcatttcaccgctacactaggaattccgcttacctctccagcactctagccaaacagtttcaaaagcagtcccggggttgagccccagcctttcacttctgacttgcttagccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcactcacgcggcgtcgctgcatcagggtttcccccattgtgcaatattccccactgctgcctcccgta

SEQ ID NO: 60 - DNA, 523 bp, Artificial Sequence
  Description of Artificial Sequence Synthetic polynucleotide
  modified_base (7)..(8): a, c, t, g, unknown or other
  modified_base (19)..(19): a, c, t, g, unknown or other
  modified_base (380)..(380): a, c, t, g, unknown or other
  modified_base (490)..(490): a, c, t, g, unknown or other
gccttgnnggctataatgnttccactatattcatttgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttgctgtatatcgcaaacagcgagtattcatcgtttactgtgtggactaccagggtatctaatcctgtttgatacccacactttcgagcatcagcgtcagttacagtccagtaagctgccttcgcaatcggagttcttcgtgatatctaagcatttcaccgctacaccacgaattccgcctacctctactgcactcnaagacgaccagtatcaactgcaattttacggttgagccgcaaactttcacagctgacttaatagtccgcctacgctccctttaaacccaataaatccggataacgctcgnatcctccgtatttaccggcggctgctggcacgg

SEQ ID NO: 61 - DNA, 662 bp, Artificial Sequence
  Description of Artificial Sequence Synthetic polynucleotide
  modified_base (328)..(328): a, c, t, g, unknown or other
  modified_base (330)..(330): a, c, t, g, unknown or other
  modified_base (332)..(332): a, c, t, g, unknown or other
  modified_base (334)..(334): a, c, t, g, unknown or other
  modified_base (336)..(337): a, c, t, g, unknown or other
acctaatgcatctctgcttcgttagtgggatgtcaagccctggtaaggttcttcgcgttgcttcgaattaaaccacatactccaccgcttgtgcgggcccccgtcaattcctttgagtttcaaccttgcgatcgtactccccaggtgggatacttattgtgttaactgcggcacggagggggtcagaccccccacacctagtatccatcgtttacggcgtggactaccagggtatctaatcctgtttgctccccacgctttcgcgcctcagcgtcagttactgtccagcaatccgccttcgccactggtgttcctccgtatatctacncntntnanngctacacacggaattccgattgcctctccagcactcaagaactacagtttcaaatgcaggctggaggttgagcccccagttttcacatctgacttgcaatcccgcctacacgccctttacacccagtaaatccggataacgcttgccacctacgtattaccgcggctgctggcacgtagttagccgtggcttattcgtcaggtaccgtcatttgtttcgtccctgacaaaagaagtttacaacccgaaagccttcttccttcacgcggcgttgctgggtcaggcttgcgcccattgcccaatattccccactgctgcctcccgta

SEQ ID NO: 62 - DNA, 685 bp, Artificial Sequence
  Description of Artificial Sequence Synthetic polynucleotide
  modified_base (27)..(28): a, c, t, g, unknown or other
  modified_base (30)..(30): a, c, t, g, unknown or other
  modified_base (143)..(143): a, c, t, g, unknown or other
accacctgtcaccgatgttccgaagannancttccattacgaagcggtcatcgggatgtcaagatcaggtaaggttcttcgcgttgcttcgaattaaaccacatgctccaccgcttgtgcgggtccccgtcaattcctttgantttcattcttgcgaacgtactccccaggtggactgcttattgcgttagctgcggcaccgaatggctttgccacccgacacctagcagtcatcgtttacggcgtggactaccagggtatctaatcctgtttgctccccacgctttcgagcctcaacgtcagtcatcgtccagcaagccgccttcgccactggtgttcctcctaatatctacgcatttcaccgctacactaggaattccacttgcctctccgacactctagctcagcagttccaaatgcagtcccggggttgagccccgggctttcacatctggcttgccgtgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcagagtttcctccattgtgcaatattccccactgctgcctcccgta

SEQ ID NO: 63 - DNA, 681 bp, Artificial Sequence
  Description of Artificial Sequence Synthetic polynucleotide
  modified_base (6)..(6): a, c, t, g, unknown or other
  modified_base (184)..(186): a, c, t, g, unknown or other
  modified_base (188)..(188): a, c, t, g, unknown or other
tgcacnacctgtcttgcctgtcccgaaggaaggtgacgttactcaccggtcaggcagatgtcaagacttggtaaggttcttcgcgttgcttcgaattaaaccacatgctccaccgcttgtgcgggtccccgtcaattcctttgagtttcattcttgcgaacgtactccccaggtggaatacttnnngngtttgcggcggcaccgaagagctgtgctccccgacacctagtattcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacccctccggcactcaagcttaacagtttccaatgcagtcccggggttaagccccagcctttcacatcagacttgttatgccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattccccactgctgcct

SEQ ID NO: 64 - DNA, 663 bp, Artificial Sequence
  Description of Artificial Sequence Synthetic polynucleotide
gccgtatctctacgaccgtcgggaacatgtcaagcccaggtaaggttcttcgcgttgcatcgaattaatccgcatgctccgccgcttgtgcgggcccccgtcaatttctttgagttttagccttgcggccgtactccccaggcgggatgcttaacgcgttagctccgacacggaacccgtggaacgggccccacatccagcatccaccgtttacggcgtggactaccagggtatctaatcctgttcgctccccacgctttcgctcctcagcgtcagtaacggcccagagacctgccttcgccattggtgttcttcccgatatctacacattccaccgttacaccgggaattccagtctcccctaccgcactcaagcccgcccgtacccggcgcggatccaccgttaagcgatggactttcacaccggacgcgacgaaccgcctacgagccctttacgcccaataattccggataacgcttgcaccctacgtattaccgcggctgctggcacgtagttagccggtgcttattcaacgggtaaactcactctcgcttgctccccgataaaagaggtttacaacccgaaggcctccatccctcacgcggcgtcgctgcatcaggcttgcgcccattgtgcaatattccccactgctgcctcccgta

SEQ ID NO: 65 - DNA, 536 bp, Artificial Sequence
  Description of Artificial Sequence Synthetic polynucleotide
tcttgcgaacgtactccccaggtggactacttattgcgtttgctgcggcaccgaagagctttgctccccgacacctagtagtcatcgtttacggcgtggactaccagggtatctaatcctgtttgctccccacgctttcgagcctcaacgtcagtcatcgtccagtaagccgccttcgccactggtgttcctcccaatatctacgcatttcaccgctacactgggaattccacttacctctccgacactctagctgcatagtttccaaagcagtcccggggttgagccccgggctttcacttcagacttacacagccgtctacgctccctttacacccagtaaatccggataacgcttgcaccatacgtattaccgcggctgctggcacgtatttagccggtgcttcttagtcaggtaccgtcattttcttccctgctgatagagctttacataccgaaatacttcttcgctcacgcggcgtcgctgcatcagggtttcccccattgtgcaatattccccactgctgcctcccgta

SEQ ID NO: 66 - DNA, 653 bp, Artificial Sequence
  Description of Artificial Sequence Synthetic polynucleotide
aaggacacattactgtccggtcagtgggatgtcaagacttggtaaggttcttcgcgttgcttcgaattaaaccacatgctccaccgcttgtgcgggtccccgtcaattcctttgagtttcattcttgcgaacgtactccccaggtggaatacttattgcgtttgctgcggcaccgaagcccttatgggccccgacacctagtattcatcgtttacggcgtggactaccagggtatctaatcctgtttgctccccacgctttcgagcctcagtgtcagttacagtccagtgagccgccttcgccactggtgttcctcctaatatctacgcatttcaccgctacactaggaattccactcacccctcctgcactccagccttacagtttcaaaagcagttccggggttgagccccggattttcacttctgacttgcatggccacctacactccctttacacccagtaaatccggataacgcttgctccatacgtattaccgcggctgctggcacgtatttagccggagcttcttagtcaggtaccgtcactatcttccctgctgatagagctttacataacgaattacttcttcactcacgcggcgtcgctgcatcagagtttcctccattgtgcaatattccccactgctgcct

SEQ ID NO: 67 - DNA, 675 bp, Artificial Sequence
  Description of Artificial Sequence Synthetic polynucleotide
  modified_base (632)..(632): a, c, t, g, unknown or other
tctctcaggtgatcaaaagatgtcaagacctggtaaggttcttcgcgttgcttcgaattaaaccacatgctccaccgcttgtgcgggcccccgtcaattcctttgagtttcaaccttgcggtcgtactccccaggcggaatgcttaatgcgttagctgcggcactgaagggcggaaaccctccaacacctagcattcatcgtttacggcatggactaccagggtatctaatcctgttcgctacccatgctttcgagcctcagcgtcagttacagaccagacagccgccttcgccactggtgttcttccatatatctacgcatttcaccgctacacatggagttccactgtcctcttctgcactcaagtttcccagtttccgatgcgcttcctcggttaagccgagggctttcacatcagacttaaaaaaccgcctgcgctcgctttacgcccaataaatccggataacgcttgccacctacgtattaccgcggctgctggcacgtagttagccgtggctttctggttggataccgtcacgccgacaacagttactctgccgaccattcttctccaacaacagagttttacgacccgaaagccttcttcactcacgcggcgttgctccatcanacttgcgtccattgtggaagattccctactgctgcctcccgta

SEQ ID NO: 68 - DNA, 634 bp, Artificial Sequence
  Description of Artificial Sequence Synthetic polynucleotide
  modified_base (5)..(5): a, c, t, g, unknown or other
  modified_base (20)..(21): a, c, t, g, unknown or other
  modified_base (145)..(145): a, c, t, g, unknown or other
  modified_base (159)..(159): a, c, t, g, unknown or other
  modified_base (403)..(403): a, c, t, g, unknown or other
  modified_base (414)..(414): a, c, t, g, unknown or other
  modified_base (441)..(441): a, c, t, g, unknown or other
  modified_base (446)..(446): a, c, t, g, unknown or other
  modified_base (491)..(491): a, c, t, g, unknown or other
  modified_base (501)..(502): a, c, t, g, unknown or other
  modified_base (510)..(510): a, c, t, g, unknown or other
  modified_base (517)..(517): a, c, t, g, unknown or other
  modified_base (526)..(526): a, c, t, g, unknown or other
  modified_base (551)..(551): a, c, t, g, unknown or other
  modified_base (564)..(564): a, c, t, g, unknown or other
  modified_base (586)..(586): a, c, t, g, unknown or other
  modified_base (597)..(597): a, c, t, g, unknown or other
  modified_base (601)..(601): a, c, t, g, unknown or other
  modified_base (605)..(605): a, c, t, g, unknown or other
aaggnaacattacttgccgnncagggagatgtcaagagcaggtaaggttcttcgcgttgcttcgaattaaaccacatgctccaccgcttgtgcgggtccccgtcaattcctttgagtttcattcttgcgaacgtactccccaggnggactacttattgngttggctgcggcaccgaatagctctgctacccgacacctagtagtcatcgtttacggcgtggactaccagggtatctaatcctgtttgctccccacgctttcgagcctcaacgtcagtcatcgtccagcaagccgccttcgccactggtgttcctcctaatatctacgcatttcaccgctacactaggaattccacttgcctctccgacactctagttcgacagtttccaatgcagtcccaggnttgagccctgntctttcacatcagacttgccataccgnctacnctccctttacacccagtaaatccggataacgcttgccccctacgnattaccgcgnntgctgggnacgtagntagccgggncttcttagtcaggtaccgtcatttncttccctgctganagaagtttacataccgaaatanttcatccttcncgcngcgncgctgcatcagggttttcccccattgtgc

SEQ ID NO: 69 - DNA, 682 bp, Artificial Sequence
  Description of Artificial Sequence Synthetic polynucleotide
acctgtcaccgatgttccgaagaaaagcttccattacgaagcggtcatcgggatgtcaagatcaggtaaggttcttcgcgttgcttcgaattaaaccacatgctccaccgcttgtgcgggtccccgtcaattcctttgagtttcattcttgcgaacgtactccccaggtggactgcttattgcgttagctgcggcaccgaatagctttgctacccgacacctagcagtcatcgtttacggcgtggactaccagggtatctaatcctgtttgctccccacgctttcgagcctcaacgtcagtcatcgtccagtaagccgccttcgccactggtgttcctcctaatatctacgcatttcaccgctacactaggaattccacttacctctccgacactctagctgcacagtttccaaagcagtccacaggttgagcccatgcctttcacttcagacttgcacagccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcaggctttcgcccattgtgcaatattccccactgctgcctcccgta

SEQ ID NO: 70 - DNA, 654 bp, Artificial Sequence
  Description of Artificial Sequence Synthetic polynucleotide
  modified_base (556)..(556): a, c, t, g, unknown or other
gtggatgtcaagaccaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccacgcctcaagggcacaacctccaagtcgacatcgtttacggcgtggactaccagggtatctaatcctgtttgctccccacgctttcgcacctgagcgtcagtcttcgtccagggggccgccttcgccaccggtattcctccagatctctacgcatttcaccgctacacctggaattctacccccctctacgagactcaagcttgccagtatcagatgcagttcccaggttgagcccggggatttcacatctgacttaacaaaccgcctgcgtgcgctttacgcccagtaattccgattaacgcttgcaccctccgtattaccgcggctgctggcacggagttagccggtgcttcttctgcgggtaacgtcaatgagcaaaggtattaactttactcccttcctcnccgctgaaagtactttacaaccccgaaggccttcttcatacacgcggcatggctgcatcaggcttgcgcccattgtgcaatattccccactgctgcct

SEQ ID NO: 71 - DNA, 636 bp, Artificial Sequence
  Description of Artificial Sequence Synthetic polynucleotide
agaaggatgtcaagaccaggtaaggttcttcgcgttgcttcgaattaaaccacatgctccaccgcttgtgcgggtccccgtcaattcctttgagtttcattcttgcgaacgtactccccaggtggaatacttattgcgttggctgcggcaccgaagcctctacggccccgacacctagtattcatcgtttacggcgtggactaccagggtatctaatcctgtttgctccccacgctttcgtgcctcagtgtcagtttcagtccagtaagccgccttcgccactgatgttcctcctaatatctacgcatttcaccgctacactaggaattccgcttacctctcctgcactccagtctgacagtttcaaaagcagtcccagagttaagccctgggttttcacttctgacttgccataccacctacgcaccctttacacccagtaattccggataacgcttgccccctacgtattaccgcggctgctggcacgtagttagccggggcttcttagtcaggtaccgtcattttcttccctgctgatagagctttacataccgaaatacttcttcactcacgcggcgtcgctgcatcagggttccccccattgtgcaatattccccactgctgcctcccgta

SEQ ID NO: 72 - DNA, 628 bp, Artificial Sequence
  Description of Artificial Sequence Synthetic polynucleotide
  modified_base (153)..(153): a, c, t, g, unknown or other
gtcaagacttggtaaggttcttcgcgttgcttcgaattaaaccacatgctccaccgcttgtgcgggtccccgtcaattcctttgagtttcattcttgcgaacgtactccccaggtggaatacttactgcgtttgcgacggcaccgaagagccntgctccccgacacctagtattcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacccctccggcactcaagtatgacagtttccaatgcagtccacaggttgagcccatgcctttcacatcagacttgccacaccgtctacgctccctttacacccagtaaatccggataacgcttgccccctacgtattaccgcggctgctggcacgtagttagccggggcttcttagtcaggtaccgtcactatcttccctgctgatagaagtttacataccgagatacttcttccttcacgcggcgtcgctgcatcagggtttcccccattgtgcaatattccccactgctgcctcccgta

SEQ ID NO: 73 - DNA, 684 bp, Artificial Sequence
  Description of Artificial Sequence Synthetic polynucleotide
  modified_base (11)..(11): a, c, t, g, unknown or other
  modified_base (179)..(185): a, c, t, g, unknown or other
cacctgtcacngatgttccgaagaaagagcgcgttacgcgctttggcatcgggatgtcaagatcaggtaaggttcttcgcgttgcttcgaattaaaccacatgctccaccgcttgtgcgggtccccgtcaattcctttgagtttcattcttgcgaacgtactccccaggtggactactnnnnnnntttgctgcggcaccgaatggccttgccacccgacacctagtagtcatcgtttacggcgtggactaccagggtatctaatcctgtttgctccccacgctttcgagcctcaacgtcagtcatcgtccagcaggccgccttcgccactggtgttcctcctaatatctacgcatttcaccgctacactaggaattccgcctgcctctccgacactccagccacgcagttccaaatgcagtcccggggttgagccccgggctttcacatctggcttgcatcgccgtctacgctccctttacacccagtaaatccggataacgcttgccccctacgtattaccgcggctgctggcacgtagttagccggggcttcttagtcaggtaccgtcatcttcttccctgctgatagaagtttacataccgaaatacttcatccttcacgcggcgtcgctgcatcagggtttcccccattgtgcaatattccccactgctgccttcccgta

SEQ ID NO: 74 - DNA, 643 bp, Artificial Sequence
  Description of Artificial Sequence Synthetic polynucleotide
cggtcatcgggatgtcaaggcctggtaaggttcttcgcgttgcttcgaattaaaccacatactccactgcttgtgcgggcccccgtcaattcctttgagtttcaaccttgcggccgtactccccaggtgggatacttattgtgttaactgcggcacggaaggggtcaatacctcccacacctagtatccatcgtttacggcgtggactaccagggtatctaatcctgtttgctccccacgctttcgcgcctcagcgtcagttaatgtccagcaggccgccttcgccactggtgttcctccgtatatctacgcatttcaccgctacacacggaattccgcctgcctctccatcactcaagaccagcagttttgaaagcagttcgggggttaagcccccggatttcacttccaacttgccggcccgcctgcgcgccctttacacccagtaaatccggataacgcttgctccctacgtattaccgcggctgctggcacgtagttagccggagcttattcttcaggtaccgtcatgtttcgtccctgattaaagatctttacaacccgaaggccttcttcaatcacgcggcgttgctgcgtcagggtttcccccattgcgcaatattccccactgctgcctcccgta

SEQ ID NO: 75 - DNA, 645 bp, Artificial Sequence
  Description of Artificial Sequence Synthetic polynucleotide
tatttctatgtgcgtcgcaggatgtcaagacttggtaaggttcttcgcgttgcgtcgaattaaaccacatactccactgcttgtgcgggcccccgtcaattcctttgagtttcaaccttgcggtcgtactccccaggtggattacttattgtgttaactgcggcactgaaggggtcaatcctccaacacctagtaatcatcgtttacggtgtggactaccagggtatctaatcctgtttgctacccacactttcgagcctcagcgtcagttggtgcccagtaggccgccttcgccactggtgttcctcccgatatctacgcattccaccgctacaccgggaattccgcctacctctgcactactcaagaaaaacagttttgaaagcagtttatgggttgagcccatagatttcacttccaacttgtcttcccgcctgcgctccctttacacccagtaattccggacaacgcttgtgacctacgttttaccgcggctgctggcacgtagttagccgtcacttccttgttgagtaccgtcattatcttcctcaacaacaggagtttacaatccgaagaccttcttcctccacgcggcgtcgctgcatcagggtttcccccattgtgcaatattccccactgctgcct

SEQ ID NO: 76 - DNA, 642 bp, Artificial Sequence
  Description of Artificial Sequence Synthetic polynucleotide
ttgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagtgacagtctagtgagctgccttcgcaatcggagttcttcgtgatatctaagcatttcaccgctacaccacgaattccgcccacctctactgtactcaagactgccagtttcaactgcaattttacggttgagccgcaaactttcacaactgacttaacaatccacctacgctccctttaaacccaataaatccggataacgctcggatcctccgtattaccgcggctgctggcacggagttagccgatccttattcatatggtacatacaaaattccacacgtggaatactttattcccatataaaagaagtttacgacccatagagccttcatccttcacgctacttgtggttcaggctctcgcccattgaccaatattcctcactgctgcc

SEQ ID NO: 77 - DNA, 683 bp, Artificial Sequence
  Description of Artificial Sequence Synthetic polynucleotide
  modified_base (118)..(121): a, c, t, g, unknown or other
  modified_base (140)..(141): a, c, t, g, unknown or other
  modified_base (144)..(144): a, c, t, g, unknown or other
acctgtcaccggtgctccgaagagaggtccccattacgggacggtcaccgggatgtcaaggcctggtaaggttcttcgcgttgcttcgaattaaaccacatgctccaccgcttgtgcnnnnccccgtcaattcctttgannttnattcttgcgaacgtactccccaggtggaatacttattgcgtttgctgcggcaccgaagctcttatgagccccgacacctagtattcatcgtttacggcgtggactaccagggtatctaatcctgtttgctccccacgctttcgagcctcagcgtcattgtcagtccagtaagccgccttcgccactggtgttcctcctaatatctacgcatttcaccgctacactaggaattccgcttacctctcctgaaatcaagcagggcagtttcaaaagccgtcccggggttgagccccgggctttcacttctgacttgctccgccgcctacgctccctttacacccagtaaatccggataacgcttgccccctacgtattaccgcggctgctggcacgtagttagccggggcttcttagtcaggtaccgtcattatcttccctgctgatagaagtttacataccgagatacttcttccttcacgcggcgtcgctgcatcagggtttcccccattgtgcaatattccccactgctgcctcccgta

SEQ ID NO: 78 - DNA, 676 bp, Artificial Sequence
  Description of Artificial Sequence Synthetic polynucleotide
ccgccccgaagggaggccccatctctggggctgtcgggaacatgtcaagcccaggtaaggttcttcgcgttgcatcgaattaatccgcatgctccgccgcttgtgcgggcccccgtcaatttctttgagttttagccttgcggccgtactccccaggcgggatgcttaacgcgttggctccgacacggagaccgtggaatggtccccacatccagcatccaccgtttacggcgtggactaccagggtatctaatcctgttcgctccccacgctttcgctcctcagcgtcagtgacggcccagagacctgccttcgccattggtgttcttcccgatatctacacattccaccgttacaccgggaattccagtctcccctaccgcactcaagcccgcccgtacccggcgcggatccaccgttaagcgatggactttcacaccggacgcgacgaaccgcctacgagccctttacgcccaataattccggataacgcttgcaccctacgtattaccgcggctgctggcacgtagttagccggtgcttattcgaaaggtacactcaccccgaagggcttgctcccagtcaaaagcggtttacaacccgaaggccgtcatcccgcacgcggcgtcgctgcatcaggcttgcgcccattgtgcaatattccccactgctgcct

SEQ ID NO: 79 - DNA, 678 bp, Artificial Sequence
  Description of Artificial Sequence Synthetic polynucleotide
  modified_base (21)..(21): a, c, t, g, unknown or other
acctgtatgggctcctctcgngccacggggtctcccccgcttcacccatatgtcaagccctggtaaggttcttcgcgttgcttcgaattaagccacatgctccgctgcttgtgcgggcccccgtcaattcctttgagttttagccttgcggccgtactccccaggcgggacgcttaatgcgttggctgcggcacggggggatcgtccccccacacctagcgtccatcgtttacggctgggactaccagggtatctaatcctgttcgctcccccagctttcgcgcctcagcgtcggtctcggcccagagggccgccttcgccaccggtgttccacccgatatctgcgcattccaccgctacaccgggtgttccaccctcccctaccggacccaagccgcggaggttccgggggcttcggggggttgagccccccgcttcgacccccggcctgccgggccgcctacgcgcgctttacgcccaatgaatccggataacgctcgccccctacgtattaccgcggctgctggcacgtagttagccggggcttcttctgcaggtacagtcttgactcttccctgctgaaagcggtttacgacccgaaggcctccgtcccgcacgcggcgtcgctgcgtcagggttccccccattgcgcaagattccccactgctgcctcccgta

SEQ ID NO: 80 - DNA, 1490 bp, Artificial Sequence; identical to SEQ ID NO: 40
  Description of Artificial Sequence Synthetic polynucleotide
  modified_base (18)..(18): a, c, t, g, unknown or other
  modified_base (26)..(26): a, c, t, g, unknown or other

SEQ ID NO: 81 - DNA, 32 bp, Artificial Sequence
  Description of Artificial Sequence Synthetic primer
attaaccctcactaaagtacggraggcagcag

SEQ ID NO: 82 - DNA, 37 bp, Artificial Sequence
  Description of Artificial Sequence Synthetic primer
aatacgactcactatagggacracacgagctgacgac